SEQUENCE LISTING (6 sequences)

SEQ ID NO: 1 - protein, 1273 aa, Artificial Sequence
  SARS-CoV-2 spike protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - RNA, 3822 nt, Artificial Sequence
  SARS-CoV-2 spike CDS
auguucguguuccugguccuccugccccuggugagcucccagugcgugaaccucaccacccgcacgcagcugccgcccgccuacaccaacagcuucacccggggcgucuacuaccccgacaagguguuccgcuccagcgugcugcacuccacccaggaccucuuccugcccuucuucagcaacgucacgugguuccacgccauccacguguccgggaccaacggcaccaagcgguucgacaacccggugcugcccuucaacgacggggucuacuucgcgagcaccgagaaguccaacaucauccgcggcuggaucuucgggacgacccucgacagcaagacccagucccugcugaucgugaacaacgccaccaacguggucaucaaggugugcgaguuccaguucugcaacgaccccuuccucggcguguacuaccacaagaacaacaagagcuggauggaguccgaguuccgggucuacagcuccgccaacaacugcacguucgaguacgugagccagcccuuccugauggaccuggagggcaagcaggggaacuucaagaaccuccgcgaguucguguucaagaacaucgacggcuacuucaagaucuacuccaagcacaccccgaucaaccugguccgggaccugccccagggguucagcgcccucgagccccugguggaccugcccaucggcaucaacaucacccgcuuccagacccuccuggcccugcaccgguccuaccucacgccgggggacagcagcuccggcuggaccgcgggcgccgccgccuacuacgugggguaccugcagccccgcaccuuccugcucaaguacaacgagaacggcaccaucacggacgcggucgacugcgcccuggacccccugagcgagaccaagugcacccucaaguccuucaccguggagaaggggaucuaccagacgagcaacuuccgggugcagcccaccgaguccaucguccgcuucccgaacaucaccaaccugugccccuucggcgagguguucaacgccacccgguucgccagcgucuacgccuggaaccgcaagcggaucuccaacugcguggcggacuacagcgugcuguacaacuccgccagcuucuccacguucaagugcuacggcgucagccccaccaagcucaacgaccugugcuucaccaacguguacgccgacuccuucgugauccgcggggacgagguccggcagaucgcccccggccagaccgggaagaucgccgacuacaacuacaagcugccggacgacuucacgggcugcgugaucgcguggaacagcaacaaccucgacuccaaggucgggggcaacuacaacuaccuguaccgccuguuccgcaagagcaaccucaagcccuucgagcgggacaucuccaccgagaucuaccaggccggcagcacccccugcaacgggguggagggcuucaacugcuacuucccccugcaguccuacggguuccagccgaccaacggcgugggguaccagcccuaccggguggucgugcugagcuucgagcuccugcacgcccccgccacggugugcggccccaagaaguccaccaaccuggucaagaacaagugcgugaacuucaacuucaacggccucaccgggaccggcgugcugaccgagagcaacaagaaguuccugccguuccagcaguucgggcgcgacaucgccgacacgaccgacgcgguccgcgacccccagacccuggagauccucgacaucacccccugcagcuucggcgggguguccgugaucacgccgggcaccaacaccagcaaccaggucgccgugcuguaccaggacgugaacugcaccgagguccccguggccauccacgccgaccagcugacgcccaccuggcggguguacuccaccggcagcaacgucuuccagacccgcgccgggugccucaucggcgcggagcacgugaacaacuccuacgagugcgacauccccaucggggccggcaucugcgccagcuaccagacgcagaccaacuccccgcggcgcgcccggagcguggcgucccagagcaucaucgccuacaccaugucccugggcgccgagaacagcgucgccuacuccaacaacagcaucgccauccccaccaacuucacgaucuccgugaccaccgagauccugcccgugagcaugaccaagacgagcgucgacugcaccauguacaucugcggggacuccaccgagugcagcaaccuccugcugcaguacggcuccuucugcacccagcucaaccgcgcgcugacggggaucgccguggagcaggacaagaacacccaggagguguucgcccaggucaagcagaucuacaagacgcccccgaucaaggacuucggcggguucaacuucagccagauccugcccgaccccuccaagcccagcaagcgguccuucaucgaggaccuccuguucaacaaggugacccuggccgacgccggcuucaucaagcaguacggcgacugccucggggacaucgcggcccgcgaccugaucugcgcccagaaguucaacggccugaccgugcucccgccccugcugaccgacgagaugaucgcccaguacaccagcgcccuccuggcggggaccaucacguccggcuggaccuucggggccggcgccgcccugcagauccccuucgcgaugcagauggccuaccgguucaacggcaucggggucacccagaacgugcucuacgagaaccagaagcugaucgccaaccaguucaacagcgccaucggcaagauccaggacucccugagcuccaccgccagcgcgcucgggaagcugcaggacguggucaaccagaacgcccaggcccugaacacgcucgugaagcagcuguccagcaacuucggcgccaucuccagcgugcugaacgacauccucucccgccuggacccgcccgaggccgaggugcagaucgaccggcugaucaccgggcgccuccagagccugcagaccuacgugacccagcagcugauccgggcggccgagauccgcgccagcgccaaccucgccgcgaccaagauguccgagugcguccugggccagagcaagcggguggacuucugcggcaagggguaccaccugauguccuucccccagagcgccccgcacggcguggucuuccuccacgugacguacguccccgcccaggagaagaacuucaccaccgcccccgcgaucugccacgacgggaaggcccacuucccccgcgagggcguguucguguccaacgggacccacugguucgucacgcagcggaacuucuacgagccgcagaucaucaccaccgacaacaccuucgugagcggcaacugcgacguggucaucggcaucgugaacaacacgguguacgacccccugcagcccgagcuggacuccuucaaggaggagcucgacaaguacuucaagaaccacaccagccccgacgucgaccugggggacaucuccggcaucaacgccagcguggugaacauccagaaggagaucgaccgccugaacgaggucgccaagaaccucaacgagucccugaucgaccugcaggagcucgggaaguacgagcaguacaucaaguggccgugguacaucuggcugggcuucaucgccggccugaucgcgaucgugauggugaccaucaugcucugcugcaugaccagcugcugcuccugccugaaggggugcugcagcugcggcuccugcugcaaguucgacgaggacgacagcgagcccguccugaagggggugaagcuccacuacacguga

SEQ ID NO: 3 - RNA, 4039 nt, Artificial Sequence
  SARS-CoV-2 spike mRNA R9515
aggagaaagcuuaccauguucguguuccugguccuccugccccuggugagcucccagugcgugaaccucaccacccgcacgcagcugccgcccgccuacaccaacagcuucacccggggcgucuacuaccccgacaagguguuccgcuccagcgugcugcacuccacccaggaccucuuccugcccuucuucagcaacgucacgugguuccacgccauccacguguccgggaccaacggcaccaagcgguucgacaacccggugcugcccuucaacgacggggucuacuucgcgagcaccgagaaguccaacaucauccgcggcuggaucuucgggacgacccucgacagcaagacccagucccugcugaucgugaacaacgccaccaacguggucaucaaggugugcgaguuccaguucugcaacgaccccuuccucggcguguacuaccacaagaacaacaagagcuggauggaguccgaguuccgggucuacagcuccgccaacaacugcacguucgaguacgugagccagcccuuccugauggaccuggagggcaagcaggggaacuucaagaaccuccgcgaguucguguucaagaacaucgacggcuacuucaagaucuacuccaagcacaccccgaucaaccugguccgggaccugccccagggguucagcgcccucgagccccugguggaccugcccaucggcaucaacaucacccgcuuccagacccuccuggcccugcaccgguccuaccucacgccgggggacagcagcuccggcuggaccgcgggcgccgccgccuacuacgugggguaccugcagccccgcaccuuccugcucaaguacaacgagaacggcaccaucacggacgcggucgacugcgcccuggacccccugagcgagaccaagugcacccucaaguccuucaccguggagaaggggaucuaccagacgagcaacuuccgggugcagcccaccgaguccaucguccgcuucccgaacaucaccaaccugugccccuucggcgagguguucaacgccacccgguucgccagcgucuacgccuggaaccgcaagcggaucuccaacugcguggcggacuacagcgugcuguacaacuccgccagcuucuccacguucaagugcuacggcgucagccccaccaagcucaacgaccugugcuucaccaacguguacgccgacuccuucgugauccgcggggacgagguccggcagaucgcccccggccagaccgggaagaucgccgacuacaacuacaagcugccggacgacuucacgggcugcgugaucgcguggaacagcaacaaccucgacuccaaggucgggggcaacuacaacuaccuguaccgccuguuccgcaagagcaaccucaagcccuucgagcgggacaucuccaccgagaucuaccaggccggcagcacccccugcaacgggguggagggcuucaacugcuacuucccccugcaguccuacggguuccagccgaccaacggcgugggguaccagcccuaccggguggucgugcugagcuucgagcuccugcacgcccccgccacggugugcggccccaagaaguccaccaaccuggucaagaacaagugcgugaacuucaacuucaacggccucaccgggaccggcgugcugaccgagagcaacaagaaguuccugccguuccagcaguucgggcgcgacaucgccgacacgaccgacgcgguccgcgacccccagacccuggagauccucgacaucacccccugcagcuucggcgggguguccgugaucacgccgggcaccaacaccagcaaccaggucgccgugcuguaccaggacgugaacugcaccgagguccccguggccauccacgccgaccagcugacgcccaccuggcggguguacuccaccggcagcaacgucuuccagacccgcgccgggugccucaucggcgcggagcacgugaacaacuccuacgagugcgacauccccaucggggccggcaucugcgccagcuaccagacgcagaccaacuccccgcggcgcgcccggagcguggcgucccagagcaucaucgccuacaccaugucccugggcgccgagaacagcgucgccuacuccaacaacagcaucgccauccccaccaacuucacgaucuccgugaccaccgagauccugcccgugagcaugaccaagacgagcgucgacugcaccauguacaucugcggggacuccaccgagugcagcaaccuccugcugcaguacggcuccuucugcacccagcucaaccgcgcgcugacggggaucgccguggagcaggacaagaacacccaggagguguucgcccaggucaagcagaucuacaagacgcccccgaucaaggacuucggcggguucaacuucagccagauccugcccgaccccuccaagcccagcaagcgguccuucaucgaggaccuccuguucaacaaggugacccuggccgacgccggcuucaucaagcaguacggcgacugccucggggacaucgcggcccgcgaccugaucugcgcccagaaguucaacggccugaccgugcucccgccccugcugaccgacgagaugaucgcccaguacaccagcgcccuccuggcggggaccaucacguccggcuggaccuucggggccggcgccgcccugcagauccccuucgcgaugcagauggccuaccgguucaacggcaucggggucacccagaacgugcucuacgagaaccagaagcugaucgccaaccaguucaacagcgccaucggcaagauccaggacucccugagcuccaccgccagcgcgcucgggaagcugcaggacguggucaaccagaacgcccaggcccugaacacgcucgugaagcagcuguccagcaacuucggcgccaucuccagcgugcugaacgacauccucucccgccuggacccgcccgaggccgaggugcagaucgaccggcugaucaccgggcgccuccagagccugcagaccuacgugacccagcagcugauccgggcggccgagauccgcgccagcgccaaccucgccgcgaccaagauguccgagugcguccugggccagagcaagcggguggacuucugcggcaagggguaccaccugauguccuucccccagagcgccccgcacggcguggucuuccuccacgugacguacguccccgcccaggagaagaacuucaccaccgcccccgcgaucugccacgacgggaaggcccacuucccccgcgagggcguguucguguccaacgggacccacugguucgucacgcagcggaacuucuacgagccgcagaucaucaccaccgacaacaccuucgugagcggcaacugcgacguggucaucggcaucgugaacaacacgguguacgacccccugcagcccgagcuggacuccuucaaggaggagcucgacaaguacuucaagaaccacaccagccccgacgucgaccugggggacaucuccggcaucaacgccagcguggugaacauccagaaggagaucgaccgccugaacgaggucgccaagaaccucaacgagucccugaucgaccugcaggagcucgggaaguacgagcaguacaucaaguggccgugguacaucuggcugggcuucaucgccggccugaucgcgaucgugauggugaccaucaugcucugcugcaugaccagcugcugcuccugccugaaggggugcugcagcugcggcuccugcugcaaguucgacgaggacgacagcgagcccguccugaagggggugaagcuccacuacacgugaggacuaguuauaagacugacuagcccgaugggccucccaacgggcccuccuccccuccuugcaccgagauuaauaaaaaaaaaaaaaaaaaaaaaaaaaaaaaaaaaaaaaaaaaaaaaaaaaaaaaaaaaaaaaaaaugcaucccccccccccccccccccccccccccccccaaaggcucuuuucagagccaccagaauu

SEQ ID NO: 4 - protein, 524 aa, Artificial Sequence
  RABV-G protein
MVPQALLFVPLLVFPLCFGKFPIYTIPDKLGPWSPIDIHHLSCPNNLVVEDEGCTNLSGFSYMELKVGYISAIKMNGFTCTGVVTEAETYTNFVGYVTTTFKRKHFRPTPDACRAAYNWKMAGDPRYEESLHNPYPDYHWLRTVKTTKESLVIISPSVADLDPYDRSLHSRVFPGGNCSGVAVSSTYCSTNHDYTIWMPENPRLGMSCDIFTNSRGKRASKGSETCGFVDERGLYKSLKGACKLKLCGVLGLRLMDGTWVAMQTSNETKWCPPGQLVNLHDFRSDEIEHLVVEELVKKREECLDALESIMTTKSVSFRRLSHLRKLVPGFGKAYTIFNKTLMEADAHYKSVRTWNEIIPSKGCLRVGGRCHPHVNGVFFNGIILGPDGNVLIPEMQSSLLQQHMELLVSSVIPLMHPLADPSTVFKNGDEAEDFVEVHLPDVHERISGVDLGLPNWGKYVLLSAGALTALMLIIFLMTCWRRVNRSEPTQHNLRGTGREVSVTPQSGKIISSWESYKSGGETGL

SEQ ID NO: 5 - RNA, 1575 nt, Artificial Sequence
  RABV-G CDS
auggugccccaggcccugcucuucgucccgcugcugguguucccccucugcuucggcaaguuccccaucuacaccauccccgacaagcuggggccguggagccccaucgacauccaccaccuguccugccccaacaaccucguggucgaggacgagggcugcaccaaccugagcggguucuccuacauggagcugaaggugggcuacaucagcgccaucaagaugaacggguucacgugcaccggcguggucaccgaggcggagaccuacacgaacuucgugggcuacgugaccaccaccuucaagcggaagcacuuccgccccacgccggacgccugccgggccgccuacaacuggaagauggccggggacccccgcuacgaggagucccuccacaaccccuaccccgacuaccacuggcugcggaccgucaagaccaccaaggagagccuggugaucaucuccccgagcguggcggaccucgaccccuacgaccgcucccugcacagccgggucuuccccggcgggaacugcuccggcguggccgugagcuccacguacugcagcaccaaccacgacuacaccaucuggaugcccgagaacccgcgccuggggauguccugcgacaucuucaccaacagccggggcaagcgcgccuccaagggcagcgagacgugcggguucgucgacgagcggggccucuacaagucccugaagggggccugcaagcugaagcucugcggcgugcugggccugcgccucauggacgggaccuggguggcgaugcagaccagcaacgagaccaaguggugcccccccggccagcuggucaaccugcacgacuuccggagcgacgagaucgagcaccucgugguggaggagcuggucaagaagcgcgaggagugccuggacgcccucgaguccaucaugacgaccaagagcguguccuuccggcgccugagccaccugcggaagcucgugcccggguucggcaaggccuacaccaucuucaacaagacccugauggaggccgacgcccacuacaaguccguccgcacguggaacgagaucaucccgagcaaggggugccugcgggugggcggccgcugccacccccacgucaacgggguguucuucaacggcaucauccucgggcccgacggcaacgugcugauccccgagaugcaguccagccugcuccagcagcacauggagcugcuggucuccagcgugaucccgcucaugcacccccuggcggaccccuccaccguguucaagaacggggacgaggccgaggacuucgucgaggugcaccugcccgacgugcacgagcggaucagcggcgucgaccucggccugccgaacugggggaaguacgugcugcucuccgccggcgcccugaccgcccugaugcugaucaucuuccucaugaccugcuggcgccgggugaaccggagcgagcccacgcagcacaaccugcgcgggaccggccgggaggucuccgugaccccgcagagcgggaagaucaucuccagcugggaguccuacaagagcggcggcgagaccgggcuguga

SEQ ID NO: 6 - RNA, 1792 nt, Artificial Sequence
  RABV-G mRNA R1803
gggagaaagcuuaccauggugccccaggcccugcucuucgucccgcugcugguguucccccucugcuucggcaaguuccccaucuacaccauccccgacaagcuggggccguggagccccaucgacauccaccaccuguccugccccaacaaccucguggucgaggacgagggcugcaccaaccugagcggguucuccuacauggagcugaaggugggcuacaucagcgccaucaagaugaacggguucacgugcaccggcguggucaccgaggcggagaccuacacgaacuucgugggcuacgugaccaccaccuucaagcggaagcacuuccgccccacgccggacgccugccgggccgccuacaacuggaagauggccggggacccccgcuacgaggagucccuccacaaccccuaccccgacuaccacuggcugcggaccgucaagaccaccaaggagagccuggugaucaucuccccgagcguggcggaccucgaccccuacgaccgcucccugcacagccgggucuuccccggcgggaacugcuccggcguggccgugagcuccacguacugcagcaccaaccacgacuacaccaucuggaugcccgagaacccgcgccuggggauguccugcgacaucuucaccaacagccggggcaagcgcgccuccaagggcagcgagacgugcggguucgucgacgagcggggccucuacaagucccugaagggggccugcaagcugaagcucugcggcgugcugggccugcgccucauggacgggaccuggguggcgaugcagaccagcaacgagaccaaguggugcccccccggccagcuggucaaccugcacgacuuccggagcgacgagaucgagcaccucgugguggaggagcuggucaagaagcgcgaggagugccuggacgcccucgaguccaucaugacgaccaagagcguguccuuccggcgccugagccaccugcggaagcucgugcccggguucggcaaggccuacaccaucuucaacaagacccugauggaggccgacgcccacuacaaguccguccgcacguggaacgagaucaucccgagcaaggggugccugcgggugggcggccgcugccacccccacgucaacgggguguucuucaacggcaucauccucgggcccgacggcaacgugcugauccccgagaugcaguccagccugcuccagcagcacauggagcugcuggucuccagcgugaucccgcucaugcacccccuggcggaccccuccaccguguucaagaacggggacgaggccgaggacuucgucgaggugcaccugcccgacgugcacgagcggaucagcggcgucgaccucggccugccgaacugggggaaguacgugcugcucuccgccggcgcccugaccgcccugaugcugaucaucuuccucaugaccugcuggcgccgggugaaccggagcgagcccacgcagcacaaccugcgcgggaccggccgggaggucuccgugaccccgcagagcgggaagaucaucuccagcugggaguccuacaagagcggcggcgagaccgggcugugaggacuaguuauaagacugacuagcccgaugggccucccaacgggcccuccuccccuccuugcaccgagauuaauaaaaaaaaaaaaaaaaaaaaaaaaaaaaaaaaaaaaaaaaaaaaaaaaaaaaaaaaaaaaaaaaugcaucccccccccccccccccccccccccccccccaaaggcucuuuucagagccaccagaauu

(19) United States
(12) Patent Application Publication
(10) Pub. No.: US 2024/0299309 A1
(43) Pub. Date: Sep. 12, 2024

(54) PHARMACEUTICAL COMPOSITION COMPRISING LIPID-BASED CARRIERS ENCAPSULATING RNA FOR MULTIDOSE ADMINISTRATION

(71) Applicant: CureVac SE, Tübingen (DE)

(72) Inventors: Michael SONNTAG, Tübingen (DE); Tilmann ROOS, Tübingen (DE); Stefan ARNOLD, Tübingen (DE); Christoph ERBACHER, Tübingen (DE)

(73) Assignee: CureVac SE, Tübingen (DE)

(21) Appl. No.: 18/258,552

(22) PCT Filed: Dec. 13, 2021
(86) PCT No.: PCT/EP2021/085439

§ 371 Date: Jun. 20, 2023

(30) Foreign Application Priority Data
Dec. 22, 2020 (WO) PCT/EP2020/087694

(51) Int. Cl. A61K 9/51; A61K 39/00; A61K 39/215; A61P 37/04

(52) U.S. Cl. CPC A61K 9/5123; A61K 39/215; A61P 37/04; A61K 2039/545

(57) ABSTRACT

The invention is inter alia directed to a pharmaceutical composition or vaccine for multidose administration comprising lipid-based carriers encapsulating an RNA, wherein the composition comprises at least one antimicrobial preservative selected from an aromatic alcohol, a sugar alcohol, thiomersal, or a combination thereof. The present invention is also directed to a kit or kit of parts for preparing and/or administering the pharmaceutical composition or vaccine for multidose administration. Also provided are methods of treating or preventing a disorders or a diseases, and first and second medical uses of the pharmaceutical composition or vaccine. Further provided is the use of aromatic alcohols, sugar alcohols, and/or thiomersal for preserving and/or preparing a composition or vaccine comprising lipid-based carriers encapsulating an RNA.

### INTRODUCTION

The present invention is inter alia directed to a pharmaceutical composition or vaccine suitable for multidose administration comprising lipid-based carriers encapsulating an RNA, wherein the composition comprises at least one antimicrobial preservative selected from at least one aromatic alcohol, at least one sugar alcohol, thiomersal, or a combination thereof.

The present invention is furthermore directed to a kit or kit of parts for preparing and/or administering the pharmaceutical composition or vaccine for multidose administration. The invention also concerns methods of treating or preventing a disorders or a diseases, and first and second medical uses of the pharmaceutical composition or vaccine. Further provided is the use of aromatic alcohols, sugar alcohols, and/or for preserving or preparing a composition or vaccine comprising lipid-based carriers encapsulating an RNA.

Therapeutic nucleic acids including RNA molecules represent an emerging class of drugs. RNA-based therapeutics include mRNA molecules encoding antigens for use as vaccines. In addition, it is envisioned to use RNA molecules for replacement therapies, e.g. providing missing proteins such as growth factors or enzymes to patients. Furthermore, the therapeutic use of noncoding RNAs such as microRNAs and RNAs suitable for genome editing (e.g. CRISPR/Cas9 guide RNAs) is considered. Accordingly, RNA-based therapeutics with the use in immunotherapy, gene therapy, and vaccination belong to the most promising and quickly developing therapeutic fields in modern medicine. For being effective, RNA is typically delivered by lipid-based carrier systems including for example liposomes and lipid nanoparticles.

For various medical applications, the provision of pharmaceutical compositions for multidose administration may be advantageous. For example, in case of a vaccine, it would be beneficial to provide a vaccine as a multidose composition to allow fast and efficient vaccination campaigns. However, multidose compositions or vaccines can only be used for a limited timespan (e.g. up to 6 hours) to e.g. avoid a bacterial contamination. By adding antimicrobial preservatives, the timespan of usage of a multidose compositions comprising lipid-based carrier encapsulating RNA could theoretically be increased. However, as lipid-based carriers encapsulating RNA represent a novel class of drug, it is unclear whether and how such drugs can be combined with antimicrobial preservatives without a negative impact on the physio-chemical and/or functional properties of the RNA and/or the lipid-based carrier. Moreover, it is unclear whether the lipid-based carrier and/or the RNA may itself have a negative impact on the effectiveness of antimicrobial preservatives.

Accordingly, there is a need in the art to provide pharmaceutical compositions of lipid-based carriers encapsulating RNA that can be used as a multidose composition for a prolonged timespan. Such a pharmaceutical compositions of lipid carriers encapsulating RNA for multidose administration could simplify the distribution of said composition, in particular in the context of a global pandemic outbreak. The underlying object is therefore to provide pharmaceutical compositions or vaccines of lipid-based carriers encapsulating RNA that can be used for a prolonged timespan in a multidose format. In particular, and object is to provide a pharmaceutical composition of lipid-based carriers encapsulating RNA wherein both the RNA cargo and the lipid-based carriers essentially maintain their physio-chemical and functional properties in the presence of an added antimicrobial preservative.

As further defined in the claims and the underlying description, the objects of the invention are inter alia solved by providing a pharmaceutical composition of lipid-based carriers encapsulating RNA, wherein the composition comprises at least one antimicrobial preservative.

### SHORT DESCRIPTION OF THE INVENTION

Typically, a pharmaceutical composition can be used for around 6 hours maximum when used in a multidose setting (e.g., several withdrawals of a dose from one vial comprising the composition). After that defined timespan, the pharmaceutical composition has to be discarded because sterility of the composition can not be guaranteed.

For facilitating a prolonged usage of a multidose composition, it is required to add an antimicrobial preservative. However, for lipid-based formulations encapsulating an RNA, a suitable antimicrobial preservative has not been described in the art. It is desired that both the RNA cargo and the lipid-based carriers essentially maintain their physio-chemical and functional properties in the presence of an added antimicrobial preservative. In addition, it is desired that the antimicrobial preservative as such is effective in the presence of the components of the multidose composition, that is lipid-based carriers encapsulating RNA.

The inventors unexpectedly identified suitable antimicrobial preservatives that may be combined with a composition comprising lipid-based carriers encapsulating an RNA. As shown in the example section, the inventors demonstrate that a set of antimicrobial preservatives was compatible with a pharmaceutical composition comprising lipid-based carriers encapsulating an RNA. The inventors show that said set of suitable antimicrobial preservatives did not affect the physio-chemical and functional properties of the pharmaceutical composition. Additionally, the inventors show that said set of suitable antimicrobial preservatives was still functional according to antimicrobial effectiveness studies. Among the suitable antimicrobial preservatives were aromatic alcohols, sugar alcohols, and thiomersal. Phenol-based antimicrobial preservatives did affect the physio-chemical and functional properties of the pharmaceutical composition and were identified to be not suitable. As shown in the example section, particularly suitable antimicrobial preservatives are aromatic alcohols (e.g. Phenoxyethanol). Notably, the suitable antimicrobial preservatives including aromatic alcohols do not have a negative effect on the innate or the adaptive immune response of an RNA-based vaccine (see Example 5).

In a first aspect, the present invention provides a pharmaceutical composition for multidose administration comprising lipid-based carriers encapsulating an RNA, wherein the composition comprises at least one antimicrobial preservative selected from at least one aromatic alcohol, at least one sugar alcohol, thiomersal, or a combination thereof.

In particular, the first aspect relates to a pharmaceutical composition for multidose administration comprising lipid-based carriers encapsulating an mRNA, wherein the composition comprises at least one antimicrobial preservative selected from at least one aromatic alcohol, wherein the pharmaceutical composition comprises more than one dose.

In a second aspect, the present invention provides a vaccine for multidose administration comprising or consisting of a pharmaceutical composition for multidose administration of the first aspect.

In embodiments, the vaccine is against a Coronavirus (e.g. SARS-CoV-2). In embodiments, the vaccine is against a Rabies virus.

In a third aspect, the present invention provides a kit or kit of parts for preparing and/or administering a multidose composition or vaccine, preferably for preparing and/or administering a multidose composition or vaccine as defined in the first or second aspect.

In embodiments, the kit or kit of parts comprises the following components
(A) at least one pharmaceutical composition comprising lipid-based carriers encapsulating an RNA; and
(B) at least one sterile dilution buffer for diluting component A, wherein the sterile dilution buffer comprises at least one antimicrobial preservative selected from at least one aromatic alcohol, at least one sugar alcohol, thiomersal, or a combination thereof.

In particular, the at least one antimicrobial preservative of component A is selected from at least one aromatic alcohol.

In a fourth aspect, the present invention relates to the medical use of the pharmaceutical composition for multidose administration of the first aspect, the vaccine of the second aspect, or the kit or kit of parts for preparing and/or administering a multidose composition or vaccine of the third aspect.

In a further aspect, the invention relates to the pharmaceutical composition for multidose administration of the first aspect, the vaccine of the second aspect, or the kit or kit of parts of the third aspect for use in the treatment or prophylaxis of an infection, or of a disorder related to such an infection.

In a further aspect, the invention relates to the pharmaceutical composition for multidose administration of the first aspect, the vaccine of the second aspect, or the kit or kit of parts for preparing and/or administering a multidose composition or vaccine of the third aspect for use in the treatment or prophylaxis of a genetic disorder or condition, or for use in the treatment or prophylaxis of a protein or enzyme deficiency or protein replacement, or for use in the treatment or prophylaxis of a tumour disorder or condition.

In a further aspect, the present invention provides a method of treating or preventing a disorder wherein the method comprises applying or administering to a subject in need thereof the pharmaceutical composition for multidose administration of the first aspect, the vaccine of the second aspect, or the kit or kit of parts of the third aspect

In a further aspect, the present invention provides a method of formulating a multidose composition or vaccine.

Further, the invention relates to the use of an aromatic alcohol, thiomersal, and/or a sugar alcohol for preserving a composition comprising lipid-based carriers encapsulating an RNA.

Further, the invention relates to the use an aromatic alcohol, thiomersal, and/or a sugar alcohol for preserving and/or formulating a composition or vaccine comprising lipid-based carriers encapsulating an RNA.

A list of particularly preferred embodiments of the invention are provided in section “List of particularly preferred embodiments (items)”.

### Definitions

For the sake of clarity and readability the following definitions are provided. Any technical feature mentioned for these definitions may be read on each and every embodiment of the invention. Additional definitions and explanations may be specifically provided in the context of these embodiments.

Percentages in the context of numbers should be understood as relative to the total number of the respective items. In other cases, and unless the context dictates otherwise, percentages should be understood as percentages by weight (wt.-%).

About: The term “about” is used when determinants or values do not need to be identical, i.e. 100% the same. Accordingly, “about” means, that a determinant or values may diverge by 0.1% to 20%, preferably by 0.1% to 10%; in particular, by 0.5%, 1%, 2%, 3%, 4%, 5%, 6%, 7%, 8%, 9%, 10%, 11%, 12%, 13%, 14%, 15%, 16%, 17%, 18%, 19%, 20%. The skilled person will know that e.g. certain parameters or determinants may slightly vary based on the method how the parameter was determined. For example, if a certain determinants or value is defined herein to have e.g. a length of “about 1000 nucleotides”, the length may diverge by 0.1% to 20%, preferably by 0.1% to 10%; in particular, by 0.5%, 1%, 2%, 3%, 4%, 5%, 6%, 7%, 8%, 9%, 10%, 11%, 12%, 13%, 14%, 15%, 16%, 17%, 18%, 19%, 20%. Accordingly, the skilled person will know that in that specific example, the length may diverge by 1 to 200 nucleotides, preferably by 1 to 200 nucleotides; in particular, by 5, 10, 20, 30, 40, 50, 60, 70, 80, 90, 100, 110, 120, 130, 140, 150, 160, 170, 180, 190, 200 nucleotides.

Adaptive immune response: The term “adaptive immune response” as used herein will be recognized and understood by the person of ordinary skill in the art, and is e.g. intended to refer to an antigen-specific response of the immune system (the adaptive immune system). Antigen specificity allows for the generation of responses that are tailored to specific pathogens or pathogen-infected cells. The ability to mount these tailored responses is usually maintained in the body by “memory cells” (B-cells). In the context of the invention, the antigen is provided by an RNA encoding at least one antigenic peptide or protein derived from a pathogen (e.g. a pandemic virus).

Antigen: The term “antigen” as used herein will be recognized and understood by the person of ordinary skill in the art, and is e.g. intended to refer to a substance which may be recognized by the immune system, preferably by the adaptive immune system, and is capable of triggering an antigen-specific immune response, e.g. by formation of antibodies and/or antigen-specific T cells as part of an adaptive immune response. Typically, an antigen may be or may comprise a peptide or protein which may be presented by the MHC to T-cells. Also fragments, variants and derivatives of peptides or proteins comprising at least one epitope are understood as antigens in the context of the invention. In the context of the present invention, an antigen may be the product of translation of a provided RNA as specified herein.

Antigenic peptide or protein: The term “antigenic peptide or protein” or “immunogenic peptide or protein” will be recognized and understood by the person of ordinary skill in the art, and is e.g. intended to refer to a peptide, protein derived from a (antigenic or immunogenic) protein which stimulates the body's adaptive immune system to provide an adaptive immune response. Therefore an antigenic/immunogenic peptide or protein comprises at least one epitope (as defined herein) or antigen (as defined herein) of the protein it is derived from.

Cationic: Unless a different meaning is clear from the specific context, the term “cationic” means that the respective structure bears a positive charge, either permanently or not permanently, but in response to certain conditions such as pH. Thus, the term “cationic” covers both “permanently cationic” and “cationisable”.

Cationisable: The term “cationisable” as used herein means that a compound, or group or atom, is positively charged at a lower pH and uncharged at a higher pH of its environment. Also in non-aqueous environments where no pH value can be determined, a cationisable compound, group or atom is positively charged at a high hydrogen ion concentration and uncharged at a low concentration or activity of hydrogen ions. It depends on the individual properties of the cationisable or polycationisable compound, in particular the pKa of the respective cationisable group or atom, at which pH or hydrogen ion concentration it is charged or uncharged. In diluted aqueous environments, the fraction of cationisable compounds, groups or atoms bearing a positive charge may be estimated using the so-called Henderson-Hasselbalch equation which is well-known to a person skilled in the art. E.g., in some embodiments, if a compound or moiety is cationisable, it is preferred that it is positively charged at a pH value of about 1 to 9, preferably 4 to 9, 5 to 8 or even 6 to 8, more preferably of a pH value of or below 9, of or below 8, of or below 7, most preferably at physiological pH values, e.g. about 7.3 to 7.4, i.e. under physiological conditions, particularly under physiological salt conditions of the cell in vivo. In other embodiments, it is preferred that the cationisable compound or moiety is predominantly neutral at physiological pH values, e.g. about 7.0-7.4, but becomes positively charged at lower pH values. In some embodiments, the preferred range of pKa for the cationisable compound or moiety is about 5 to about 7.

Cationic or polycationic compound: The term “cationic or polycationic compound” as used herein will be recognized and understood by the person of ordinary skill in the art, and is for example intended to refer to a charged molecule, which is positively charged at a pH value ranging from about 1 to 9, at a pH value ranging from about 3 to 8, at a pH value ranging from about 4 to 8, at a pH value ranging from about 5 to 8, more preferably at a pH value ranging from about 6 to 8, even more preferably at a pH value ranging from about 7 to 8, most preferably at a physiological pH, e.g. ranging from about 7.2 to about 7.5. Accordingly, a cationic lipid (including lipidoids) may be any positively charged compound or polymer which is positively charged under physiological conditions.

Coding sequence/coding region: The terms “coding sequence” or “coding region” and the corresponding abbreviation “cds” as used herein will be recognized and understood by the person of ordinary skill in the art, and are e.g. intended to refer to a sequence of several nucleotide triplets, which may be translated into a peptide or protein. A coding sequence in the context of the present invention may be an RNA sequence consisting of a number of nucleotides that may be divided by three, which starts with a start codon and which preferably terminates with a stop codon.

CRISPR-associated protein: The term “CRISPR-associated protein” will be recognized and understood by the person of ordinary skill in the art. The term “CRISPR-associated protein” refers to RNA-guided endonucleases that are part of a CRISPR (Clustered Regularly Interspaced Short Palindromic Repeats) system (and their homologs, variants, fragments or derivatives), which is used by prokaryotes to confer adaptive immunity against foreign DNA elements. CRISPR-associated proteins include, without limitation, Cas9, Cpf1 (Cas12), C2c1, C2c3, C2c2, Cas13, CasX and CasY. As used herein, the term “CRISPR-associated protein” includes wild-type proteins as well as homologs, variants, fragments and derivatives thereof. Therefore, when referring to artificial nucleic acid molecules encoding Cas9, Cpf1 (Cas12), C2c1, C2c3, and C2c2, Cas13, CasX and CasY, said artificial nucleic acid molecules may encode the respective wild-type proteins, or homologs, variants, fragments and derivatives thereof. Besides Cas9 and Cas12 (Cpf1), several other CRISPR-associated protein exist that are suitable for genetic engineering in the context of the invention, including Cas13, CasX and CasY; e.g. Cas13 i.e. WP15770004, WP18451595, WP21744063, WP21746774, ERK53440, WP31473346, CVRQ01000008, CRZ35554, WP22785443, WP36091002, WP12985477, WP13443710, ETD76934, WP38617242, WP2664492, WP4343973, WP44065294, ADAR2DD, WP47447901, ERI81700, WP34542281, WP13997271, WP41989581, WP47431796, WP14084666, WP60381855, WP14165541, WP63744070, WP65213424, WP45968377, EHO06562, WP6261414, EKB06014, WP58700060, WP13446107, WP44218239, WP12458151, ERJ81987, ERJ65637, WP21665475, WP61156637, WP23846767, ERJ87335, WP5873511, WP39445055, WP52912312, WP53444417, WP12458414, WP39417390, EOA10535, WP61156470, WP13816155, WP5874195, WP39437199, WP39419792, WP39431778, WP46201018, WP39442171, WP39426176, WP39418912, WP39434803, WP39428968, WP25000926, EFU31981, WP4343581, WP36884929, BAU18623, AFJ07523, WP14708441, WP36860899, WP61868553, KJJ86756, EGQ18444, EKY00089, WP36929175, WP7412163, WP44072147, WP42518169, WP44074780, WP15024765, WP49354263, WP4919755, WP64970887, WP61710138); CasX (i.e. OGP07438, OHB99618); CasY (i.e. OJI08769, OGY82221, OJI06454, APG80656, OJI07455, OJI09436, PIP58309).

Guide RNA: As used herein, the term “guide RNA” (gRNA) thus relates to any RNA molecule capable of targeting a CRISPR-associated protein as defined above to a target DNA sequence of interest. In the context of the invention, the term guide RNA has to be understood in its broadest sense, and may comprise two-molecule gRNAs (“tracrRNA/crRNA”) comprising crRNA (“CRISPR RNA” or “targeter-RNA” or “crRNA” or “crRNA repeat”) and a corresponding tracrRNA (“trans-acting CRISPR RNA” or “activator-RNA” or “tracrRNA”) molecule, or single-molecule gRNAs. A “sgRNA” typically comprises a crRNA connected at its 3′ end to the 5′ end of a tracrRNA through a “loop” sequence.

Derived from: The term “derived from” as used throughout the present specification in the context of a nucleic acid, i.e. for a nucleic acid “derived from” (another) nucleic acid, means that the nucleic acid, which is derived from (another) nucleic acid, shares e.g. at least 60%, 70%, 80%, 81%, 82%, 83%, 84%, 85%, 86%, 87%, 88%, 89%, 90%, 91%, 92%, 93%, 94%, 95%, 96%, 97%, 98%, or 99% sequence identity with the nucleic acid from which it is derived. The skilled person is aware that sequence identity is typically calculated for the same types of nucleic acids, i.e. for DNA sequences or for RNA sequences. Thus, it is understood, if a DNA is “derived from” an RNA or if an RNA is “derived from” a DNA, in a first step the RNA sequence is converted into the corresponding DNA sequence (in particular by replacing the uracils (U) by thymidines (T) throughout the sequence) or, vice versa, the DNA sequence is converted into the corresponding RNA sequence (in particular by replacing the T by U throughout the sequence). Thereafter, the sequence identity of the DNA sequences or the sequence identity of the RNA sequences is determined. Preferably, a nucleic acid “derived from” a nucleic acid also refers to nucleic acid, which is modified in comparison to the nucleic acid from which it is derived, e.g. in order to increase RNA stability even further and/or to prolong and/or increase protein production. In the context of amino acid sequences (e.g. antigenic peptides or proteins) the term “derived from” means that the amino acid sequence, which is derived from (another) amino acid sequence, shares e.g. at least 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence from which it is derived.

Epitope: The term “epitope” (also called “antigen determinant” in the art) as used herein will be recognized and understood by the person of ordinary skill in the art, and is e.g. intended to refer to T cell epitopes and B cell epitopes. T cell epitopes or parts of the antigenic peptides or proteins and may comprise fragments preferably having a length of about 6 to about 20 or even more amino acids, e.g. fragments as processed and presented by MHC class I molecules, preferably having a length of about 8 to about 10 amino acids, e.g. 8, 9, or 10, (or even 11, or 12 amino acids), or fragments as processed and presented by MHC class II molecules, preferably having a length of about 13 to about 20 or even more amino acids. These fragments are typically recognized by T cells in form of a complex consisting of the peptide fragment and an MHC molecule, i.e. the fragments are typically not recognized in their native form. B cell epitopes are typically fragments located on the outer surface of (native) protein or peptide antigens, preferably having 5 to 15 amino acids, more preferably having 5 to 12 amino acids, even more preferably having 6 to 9 amino acids, which may be recognized by antibodies, i.e. in their native form. Such epitopes of proteins or peptides may furthermore be selected from any of the herein mentioned variants of such proteins or peptides. In this context epitopes can be conformational or discontinuous epitopes which are composed of segments of the proteins or peptides as defined herein that are discontinuous in the amino acid sequence of the proteins or peptides as defined herein but are brought together in the three-dimensional structure or continuous or linear epitopes which are composed of a single polypeptide chain.

Fragment: The term “fragment” as used throughout the present specification in the context of a nucleic acid sequence (e.g. RNA or a DNA) or an amino acid sequence may typically be a shorter portion of a full-length sequence of e.g. a nucleic acid sequence or an amino acid sequence. Accordingly, a fragment, typically, consists of a sequence that is identical to the corresponding stretch within the full-length sequence. A preferred fragment of a sequence in the context of the present invention, consists of a continuous stretch of entities, such as nucleotides or amino acids corresponding to a continuous stretch of entities in the molecule the fragment is derived from, which represents at least 40%, 50%, 60%, 70%, 80%, 90%, 95% of the total (i.e. full-length) molecule from which the fragment is derived (e.g. a virus protein). The term “fragment” as used throughout the present specification in the context of proteins or peptides may, typically, comprise a sequence of a protein or peptide as defined herein, which is, with regard to its amino acid sequence, N-terminally and/or C-terminally truncated compared to the amino acid sequence of the original protein. Such truncation may thus occur either on the amino acid level or correspondingly on the nucleic acid level. A sequence identity with respect to such a fragment as defined herein may therefore preferably refer to the entire protein or peptide as defined herein or to the entire (coding) nucleic acid molecule of such a protein or peptide. Fragments of proteins or peptides may comprise at least one epitope of those proteins or peptides.

Heterologous: The terms “heterologous” or “heterologous sequence” as used throughout the present specification in the context of a nucleic acid sequence or an amino acid sequence refers to a sequence (e.g. RNA, DNA, amino acid) has to be understood as a sequence that is derived from another gene, another allele, or e.g. another species or virus. Two sequences are typically understood to be “heterologous” if they are not derivable from the same gene or from the same allele. I.e., although heterologous sequences may be derivable from the same organism or virus, in nature, they do not occur in the same nucleic acid or protein.

Humoral immune response: The terms “humoral immunity” or “humoral immune response” will be recognized and understood by the person of ordinary skill in the art, and are e.g. intended to refer to B-cell mediated antibody production and optionally to accessory processes accompanying antibody production. A humoral immune response may be typically characterized, e.g. by Th2 activation and cytokine production, germinal center formation and isotype switching, affinity maturation and memory cell generation. Humoral immunity may also refer to the effector functions of antibodies, which include pathogen and toxin neutralization, classical complement activation, and opsonin promotion of phagocytosis and pathogen elimination.

Identity (of a sequence): The term “identity” as used throughout the present specification in the context of a nucleic acid sequence or an amino acid sequence will be recognized and understood by the person of ordinary skill in the art, and is e.g. intended to refer to the percentage to which two sequences are identical. To determine the percentage to which two sequences are identical, e.g. nucleic acid sequences or amino acid (aa) sequences as defined herein, preferably the aa sequences encoded by the nucleic acid sequence as defined herein or the aa sequences themselves, the sequences can be aligned in order to be subsequently compared to one another. Therefore, e.g. a position of a first sequence may be compared with the corresponding position of the second sequence. If a position in the first sequence is occupied by the same residue as is the case at a position in the second sequence, the two sequences are identical at this position. If this is not the case, the sequences differ at this position. If insertions occur in the second sequence in comparison to the first sequence, gaps can be inserted into the first sequence to allow a further alignment. If deletions occur in the second sequence in comparison to the first sequence, gaps can be inserted into the second sequence to allow a further alignment. The percentage to which two sequences are identical is then a function of the number of identical positions divided by the total number of positions including those positions which are only occupied in one sequence. The percentage to which two sequences are identical can be determined using an algorithm, e.g. an algorithm integrated in the BLAST program.

Immunogen, immunogenic: The terms “immunogen” or “immunogenic” will be recognized and understood by the person of ordinary skill in the art, and are e.g. intended to refer to a compound that is able to stimulate/induce an immune response. Preferably, an immunogen is a peptide, polypeptide, or protein. An immunogen in the sense of the present invention is the product of translation of a provided nucleic acid, comprising at least one coding sequence encoding at least one antigenic peptide, protein derived from e.g. a coronavirus protein as defined herein. Typically, an immunogen elicits an adaptive immune response.

Immune response: The term “immune response” will be recognized and understood by the person of ordinary skill in the art, and is e.g. intended to refer to a specific reaction of the adaptive immune system to a particular antigen (so called specific or adaptive immune response) or an unspecific reaction of the innate immune system (so called unspecific or innate immune response), or a combination thereof.

Immune system: The term “immune system” will be recognized and understood by the person of ordinary skill in the art, and is e.g. intended to refer to a system of the organism that protects the organisms from infection. If a pathogen succeeds in passing a physical barrier of an organism and enters this organism, the innate immune system provides an immediate non-specific response. If pathogens evade this innate response, vertebrates possess a second layer of protection, the adaptive immune system. The immune system adapts its response during an infection to improve its recognition of the pathogen. This improved response is then retained after the pathogen has been eliminated, in the form of an immunological memory, and allows the adaptive immune system to mount faster and stronger attacks each time this pathogen is encountered. According to this, the immune system comprises the innate and the adaptive immune system. Each of these two parts typically contains so called humoral and cellular components.

Innate immune system: The term “innate immune system” (also known as non-specific or unspecific immune system) will be recognized and understood by the person of ordinary skill in the art, and is e.g. intended to refer to a system typically comprising the cells and mechanisms that defend the host from infection by other organisms in a non-specific manner. This means that the cells of the innate system may recognize and respond to pathogens in a generic way, but unlike the adaptive immune system, it does not confer long-lasting or protective immunity to the host. The innate immune system may be activated by ligands of pattern recognition receptor e.g. Toll-like receptors, NOD-like receptors, or RIG-1 like receptors.

Lipidoid compound: A lipidoid compound, also simply referred to as lipidoid, is a lipid-like compound, i.e. an amphiphilic compound with lipid-like physical properties. In the context of the present invention, the term lipid is considered to encompass lipidoid compounds.

Nucleic acid, nucleic acid molecule: The terms “nucleic acid” or “nucleic acid molecule” as used herein, will be recognized and understood by the person of ordinary skill in the art. The terms “nucleic acid” or “nucleic acid molecule” preferably refers to DNA (molecules) or RNA (molecules). The term is used synonymously with the term polynucleotide. Preferably, a nucleic acid or a nucleic acid molecule is a polymer comprising or consisting of nucleotide monomers that are covalently linked to each other by phosphodiester-bonds of a sugar/phosphate-backbone. The terms “nucleic acid” or “nucleic acid molecule” also encompasses modified nucleic acid (molecules), such as base-modified, sugar-modified or backbone-modified DNA or RNA (molecules) as defined herein.

Nucleic acid sequence, DNA sequence, RNA sequence: The terms “nucleic acid sequence”, “DNA sequence”, “RNA sequence” will be recognized and understood by the person of ordinary skill in the art, and e.g. refer to a particular and individual order of the succession of its nucleotides.

Nucleic acid species: In the context of the invention, the term “nucleic acid species” is not restricted to mean “one single nucleic acid molecule” but is understood to comprise an ensemble of essentially identical nucleic acid molecules. Accordingly, it may relate to a plurality of essentially identical nucleic acid molecules, e.g. DNA or RNA molecules.

Permanently cationic: The term “permanently cationic” as used herein will be recognized and understood by the person of ordinary skill in the art, and means, e.g., that the respective compound, or group, or atom, is positively charged at any pH value or hydrogen ion activity of its environment. Typically, the positive charge results from the presence of a quaternary nitrogen atom. Where a compound carries a plurality of such positive charges, it may be referred to as permanently polycationic.

RNA: The term “RNA” is the usual abbreviation for ribonucleic acid. It is a nucleic acid molecule, i.e. a polymer consisting of nucleotide monomers. These nucleotides are usually adenosine-monophosphate (AMP), uridine-monophosphate (UMP), guanosine-monophosphate (GMP) and cytidine-monophosphate (CMP) monomers or analogs thereof, which are connected to each other along a so-called backbone. The backbone is formed by phosphodiester bonds between the sugar, i.e. ribose, of a first and a phosphate moiety of a second, adjacent monomer. The specific order of the monomers, i.e. the order of the bases linked to the sugar/phosphate-backbone, is called the RNA sequence. RNA can be obtained by transcription of a DNA sequence, e.g., inside a cell. In eukaryotic cells, transcription is typically performed inside the nucleus or the mitochondria. In vivo, transcription of DNA usually results in the so-called premature RNA which has to be processed into so-called messenger-RNA, usually abbreviated as mRNA. Processing of the premature RNA, e.g. in eukaryotic organisms, comprises a variety of different posttranscriptional modifications such as splicing, 5′-capping, polyadenylation, export from the nucleus or the mitochondria and the like. The sum of these processes is also called maturation of RNA. The mature messenger RNA usually provides the nucleotide sequence that may be translated into an amino acid sequence of a particular peptide or protein. Typically, a mature mRNA comprises a 5-cap, optionally a 5′UTR, a coding sequence, optionally a 3′UTR and a poly(A) sequence. If RNA molecules are of synthetic origin, the RNA molecules are meant not to be produced in vivo, i.e. inside a cell or purified from a cell, but in an in vitro method. An examples for a suitable in vitro method is in vitro transcription. In addition to messenger RNA, several non-coding types of RNA exist which may be involved in regulation of transcription and/or translation, and immunostimulation and which may also be produced by in vitro transcription.

RNA in vitro transcription: The terms “RNA in vitro transcription” or “in vitro transcription” relate to a process wherein RNA is synthesized in a cell-free in vitro system. RNA may be obtained by DNA-dependent in vitro transcription of an appropriate DNA template, which is typically a linear DNA template. The promoter for controlling RNA in vitro transcription can be any promoter for any DNA-dependent RNA polymerase. Reagents used in RNA in vitro transcription typically include a DNA template, ribonucleotide triphosphates, a cap analogue, a DNA-dependent RNA polymerase, a ribonuclease inhibitor, MgCl2, a buffer which can also contain antioxidants (e.g. DTT), and/or polyamines such as spermidine. After RNA transcription, the DNA template is typically removed using e.g. DNAse digestion step, followed by several purification steps.

T-cell responses: The terms “cellular immunity” or “cellular immune response” or “cellular T-cell responses” as used herein will be recognized and understood by the person of ordinary skill in the art, and are for example intended to refer to the activation of macrophages, natural killer cells (NK), antigen-specific cytotoxic T-lymphocytes, and the release of various cytokines in response to an antigen. In more general terms, cellular immunity is not based on antibodies, but on the activation of cells of the immune system. Typically, a cellular immune response may be characterized e.g. by activating antigen-specific cytotoxic T-lymphocytes that are able to induce apoptosis in cells, e.g. specific immune cells like dendritic cells or other cells, displaying epitopes of foreign antigens on their surface.

Variant (of a sequence): The term “variant” as used throughout the present specification in the context of a nucleic acid sequence will be recognized and understood by the person of ordinary skill in the art, and is e.g. intended to refer to a variant of a nucleic acid sequence derived from another nucleic acid sequence. E.g., a variant of a nucleic acid sequence may exhibit one or more nucleotide deletions, insertions, additions and/or substitutions compared to the nucleic acid sequence from which the variant is derived. A variant of a nucleic acid sequence may at least 50%, 60%, 70%, 80%, 90%, or 95% identical to the nucleic acid sequence the variant is derived from. The variant is a functional variant in the sense that the variant has retained at least 50%, 60%, 70%, 80%, 90%, or 95% or more of the function of the sequence where it is derived from. A “variant” of a nucleic acid sequence may have at least 70%, 75%, 80%, 85%, 90%, 95%, 98% or 99% nucleotide identity over a stretch of at least 10, 20, 30, 50, 75 or 100 nucleotide of such nucleic acid sequence. The term “variant” as used throughout the present specification in the context of proteins or peptides is e.g. intended to refer to a proteins or peptide variant having an amino acid sequence which differs from the original sequence in one or more mutation(s)/substitution(s), such as one or more substituted, inserted and/or deleted amino acid(s). Preferably, these fragments and/or variants have the same, or a comparable specific antigenic property (immunogenic variants, antigenic variants). Insertions and substitutions are possible, in particular, at those sequence positions which cause no modification to the three-dimensional structure or do not affect the binding region. Modifications to a three-dimensional structure by insertion(s) or deletion(s) can easily be determined e.g. using CD spectra (circular dichroism spectra). A “variant” of a protein or peptide may have at least 70%, 75%, 80%, 85%, 90%, 95%, 98% or 99% amino acid identity over a stretch of at least 10, 20, 30, 50, 75 or 100 amino acids of such protein or peptide. Preferably, a variant of a protein comprises a functional variant of the protein, which means, in the context of the invention, that the variant exerts essentially the same, or at least 40%, 50%, 60%, 70%, 80%, 90% of the immunogenicity as the protein it is derived from.

### DETAILED DESCRIPTION OF THE INVENTION

The present application is filed together with a sequence listing in electronic format, which is part of the description of the present application (WIPO standard ST.25). The information contained in the sequence listing is incorporated herein by reference in its entirety. Where reference is made herein to a “SEQ ID NO”, the corresponding nucleic acid sequence or amino acid (aa) sequence in the sequence listing having the respective identifier is referred to. For many sequences, the sequence listing also provides additional detailed information, e.g. regarding certain structural features, sequence optimizations, GenBank or NCBI identifiers, or additional detailed information regarding its coding capacity. Such information is provided under numeric identifier <223> in the WIPO standard ST.25 sequence listing. Accordingly, information provided under said numeric identifier <223> is explicitly included herein in its entirety and has to be understood as integral part of the description of the underlying invention. Where reference is made to “SEQ ID NOs” of other patent applications or patents, said sequences, e.g. amino acid sequences or nucleic acid sequences, are explicitly incorporated herein by reference. For “SEQ ID NOs” that are included by reference, information provided under identifier <223> (of the respective sequence protocol provided in the referenced application or patent) is also included herein in its entirety.

### Pharmaceutical Composition for Multidose Administration:

In a first aspect, the present invention provides a pharmaceutical composition for multidose administration comprising lipid-based carriers encapsulating an RNA, preferably mRNA, wherein the composition comprises at least one antimicrobial preservative.

In a preferred embodiment, the pharmaceutical composition provided herein is a pharmaceutical multidose composition.

The term “multidose composition” as used herein refers to a pharmaceutical composition that comprises more than one dose of an active pharmaceutical ingredient (API). In the context of the invention, the active pharmaceutical ingredient is an RNA, e.g. a therapeutic RNA, preferably mRNA. Accordingly, the pharmaceutical composition for multidose administration suitably comprises more than one dose of RNA.

As used herein, “dose” means an amount of the API, herein the lipid-based carriers encapsulating the RNA, that significantly induces a positive modification of a disease or disorder. The amount of the “dose” of the composition will vary in connection with the particular condition to be treated and also with the age and physical condition of the patient to be treated, the severity of the condition, the duration of the treatment, the nature of the accompanying therapy, of the particular pharmaceutically acceptable carrier used, and similar factors, within the knowledge and experience of the skilled person.

As used herein, the “dose” typically relates to the amount of RNA, preferably mRNA, and may be in a range of 1 ug to 200 ug RNA per dose.

In preferred embodiments, one dose comprises an amount of RNA, preferably mRNA, in a rage of 1 ug to 200 ug, preferably 5 ug to 200 ug, more preferably 5 ug to 100 ug, even more preferably 10 ug to 100 ug.

In some embodiments, the pharmaceutical multidose composition comprises 5 to 100 does, preferably 5 to 50 doses or 10 to 50 doses. In preferred embodiments, the pharmaceutical multidose composition comprises 5 to 25 does, preferably 5 to 10 doses. Accordingly, the composition may comprise 2, 3, 4, 5, 6, 7, 8, 9, 10, 11, 12, 13, 14, 15, 16, 17, 18, 19, 20, or more than 20 doses

The term “lipid-based carriers” encompass lipid based delivery systems for RNA, preferably mRNA, that comprise one or more lipid components (e.g. an aggregation reducing lipid, a cationic lipid, etc.). A lipid-based carrier may additionally comprise other components suitable for encapsulating/incorporating an RNA including a cationic or polycationic polymer, a cationic or polycationic polysaccharide, a cationic or polycationic protein, a cationic or polycationic peptide, or any combinations thereof. The term “lipid-based carriers” encompasses artificial lipid-based carrier system and does not comprise natural systems including virus particles etc.

In the context of the invention, a typical “lipid-based carrier” is selected from liposomes, lipid nanoparticles (LNPs), lipoplexes, and/or nanoliposomes. The RNA of the pharmaceutical composition for multidose administration may completely or partially incorporated or encapsulated in a lipid-based carrier, wherein the RNA may be located in the interior space of the lipid-based carrier, within the lipid layer/membrane of the lipid-based carrier, or associated with the exterior surface of the lipid-based carrier. The incorporation of an RNA into lipid-based carriers is also referred to as “encapsulation”. A “lipid-based carrier” is not restricted to any particular morphology, and include any morphology generated when e.g. an aggregation reducing lipid and at least one further lipid are combined, e.g. in an aqueous environment in the presence of an RNA. For example, an LNP, a liposome, a lipid complex, a lipoplex and the like are within the scope of the term “lipid-based carrier”. Lipid-based carriers can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 nm and 500 nm in diameter. Liposomes, a specific type of lipid-based carrier, are characterized as microscopic vesicles having an interior aqua space sequestered from an outer medium by a membrane of one or more bilayers. In a liposome, the RNA is typically located in the interior aqueous space enveloped by some or the entire lipid portion of the liposome. Bilayer membranes of liposomes are typically formed by amphiphilic molecules, such as lipids of synthetic or natural origin that comprise spatially separated hydrophilic and hydrophobic domains. Lipid nanoparticles (LNPs), a specific type of lipid-based carrier, are characterized as microscopic lipid particles having a solid core or a partially solid core. Typically, an LNP does not comprise an interior aqua space sequestered from an outer medium by a bilayer. In an LNP, the RNA may be encapsulated or incorporated in the lipid portion of the LNP enveloped by some or the entire lipid portion of the LNP. An LNP may comprise any lipid capable of forming a particle to which the RNA may be attached, or in which the RNA may be encapsulated.

Suitably, the RNA, preferably the mRNA, is encapsulated in the lipid-based carriers of the pharmaceutical composition for multidose administration.

The term “encapsulated”, e.g. incorporated, complexed, encapsulated, partially encapsulated, associated, partially associated, refers to the essentially stable combination of RNA with one or more lipids into lipid-based carriers (e.g. larger complexes or assemblies) without covalent binding of the RNA. The lipid-based carriers-encapsulated RNA may be completely or partially located in the interior of the lipid-based carrier (e.g. the lipid portion and/or an interior space) and/or within the lipid layer/membrane of the lipid-based carriers. The encapsulation of an RNA into lipid-based carriers is also referred to herein as “incorporation” as the RNA is preferably contained within the interior of the lipid-based carriers. Without wishing to be bound to theory, the purpose of incorporating or encapsulating RNA into lipid-based carriers may be to protect the RNA from an environment which may contain enzymes, chemicals, or conditions that degrade the RNA. Moreover, incorporating RNA into lipid-based carriers may promote the uptake of the RNA, and hence, may enhance the therapeutic effect of the RNA when administered to a cell or a subject.

In the context of the invention the term “antimicrobial preservative” relates to a group of compounds that can be used to prevent or reduce microbial growth in a pharmaceutical product. Accordingly, an antimicrobial preservative has a certain antimicrobial activity, e.g. bacteriostatic or bactericide activity. In the art, antimicrobial activity of preservatives is tested against the microbes listed in pharmacopeial test methods, but also possibly against product or facility-specific microbes that may pose a risk to product quality. An “antimicrobial preservative” is typically selected in a way that the compound does not adversely interact with the drug, or other components in the formulation, package or delivery device, and that the preservative maintains preservative efficacy throughout products shelf life and in use period. Typically, an antimicrobial preservative is required for multidose pharmaceutical compounds, e.g. multidose vaccines. Exemplary antimicrobial preservatives comprise benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, hexetidine, imidurea, phenol, phenoxyethanol, phenylethyl alcohol, phenylmercuric nitrate, propylene glycol, and/or thimerosal.

In preferred embodiments, the at least one antimicrobial preservative is not selected from an antimicrobial preservative that comprises a phenol or a phenol group. A phenol is a compound that comprises a hydroxyl group (—OH) that is directly bonded to an aromatic ring. Accordingly, the at least one antimicrobial preservative is not selected from antimicrobial preservatives that comprises a phenol group, e.g. chlorocresol, chloroxylenol, cresol, or phenol.

In preferred embodiments, the at least one antimicrobial preservative is not selected from an antimicrobial preservative that comprises a phenol or a phenol group, but may comprise a phenyl group.

Accordingly, in preferred embodiments, the at least one antimicrobial preservative is selected from a phenol-free antimicrobial preservative.

In embodiments, the at least one antimicrobial preservative is selected from bronopol, cetrimide, cetylpyridinium chloride, chlorhexidine, chlorobutanol, ethyl alcohol, hexetidine, imidurea, phenylmercuric nitrate, propylene glycol, a sugar alcohol, an aromatic alcohol, and/or thiomersal.

In preferred embodiments, the pharmaceutical composition for multidose administration comprising lipid-based carriers encapsulating an RNA comprises at least one antimicrobial preservative selected from at least one aromatic alcohol, at least one sugar alcohol, thiomersal, or a combination thereof.

In particularly preferred embodiments, the at least one antimicrobial preservative is selected from at least one aromatic alcohol.

The use of aromatic alcohols as antimicrobial preservative is particularly preferred in the context of the invention as aromatic alcohols show an as yet undescribed and surprising compatibility with lipid-based carriers encapsulating an RNA.

For example, physiochemical characteristics (RNA integrity, lipid-based carrier integrity) and functional characteristics (e.g. translation into protein, potency) are not impaired by aromatic alcohols.

Aromatic alcohols comprise any alcohol in which the alcoholic hydroxy group (—OH) is attached to a carbon which is itself bonded to an aromatic ring. The key difference between aromatic alcohols and phenol is that the hydroxyl group (—OH) of phenol is directly bonded to an aromatic ring.

In preferred embodiments, the at least one aromatic alcohol of the pharmaceutical composition for multidose administration is selected from benzyl alcohol, phenoxyethanol, phenylethyl alcohol, or a combination thereof.

In preferred embodiments, the least one antimicrobial preservative of the pharmaceutical composition for multidose administration is selected from at least one aromatic alcohol, wherein the at least one aromatic alcohol is phenoxyethanol.

The aromatic alcohol phenoxyethanol (2-Phenoxyethanol, 2-PE, Phenylglycol, 2-Phenoxy-1-ethanol, Monophenylglycol, Ethylenglycolmonophenylether; CAS: 122-99-6) is an organic compound having the chemical formula C8H10O2.

In preferred embodiments, the least one antimicrobial preservative of the pharmaceutical composition for multidose administration is selected from at least one aromatic alcohol, wherein the at least one aromatic alcohol is selected from benzyl alcohol.

The aromatic alcohol benzyl alcohol (Phenylmethanol, Phenylcarbinol, BnOH; CAS: 100-51-6) is an organic compound having the chemical formula C7H8O.

In embodiments, the least one antimicrobial preservative of the pharmaceutical composition for multidose administration is selected from at least one aromatic alcohol, wherein the at least one aromatic alcohol is selected from phenylethyl alcohol.

The aromatic alcohol phenylethyl alcohol (2-Phenylethanol, 2-Phenylethylalkohol, β-Phenylethylalkohol, Benzylcarbinol, Phenethanol; CAS: 60-12-8) is an organic compound having the chemical formula C8H10O.

In embodiments, the at least one aromatic alcohol (e.g. phenoxyethanol) is in a concentration of about 0.1% (w/v) to about 5% (w/v), preferably in a concentration of about 0.1% (w/v) to about 2%.

In preferred embodiments, the at least one aromatic alcohol (e.g. phenoxyethanol) is in a concentration of about 0.5% (w/v) to about 1% (w/v).

In specific embodiments, the at least one aromatic alcohol (e.g. phenoxyethanol) is in a concentration of 0.1% (w/v), 0.2% (w/v), 0.3% (w/v), 0.4% (w/v), 0.5% (w/v), 0.6% (w/v), 0.7% (w/v), 0.8% (w/v), 0.9% (w/v), 1% (w/v), 1.1% (w/v), 1.2% (w/v), 1.3% (w/v), 1.4% (w/v), 1.5% (w/v), 1.6% (w/v), 1.7% (w/v), 1.8% (w/v), 1.9% (w/v), or 2% (w/v).

In preferred embodiments, the at least one aromatic alcohol (e.g. phenoxyethanol) is in a concentration of about 0.5% (w/v).

In other preferred embodiments, the at least one aromatic alcohol (e.g. phenoxyethanol) is in a concentration of about 1% (w/v).

In preferred embodiments, the pharmaceutical composition for multidose administration comprises at least one antimicrobial preservative selected from thiomersal.

The use of thiomersal as antimicrobial preservative is particularly preferred in the context of the invention as thiomersal shows an as yet undescribed and surprising compatibility with lipid-based carriers encapsulating an RNA.

Thiomersal (thiomerosal, Natrium-2-(ethylmercurithio)benzoat; CAS: 54-64-8) is an organomercury compound having the chemical formula C9H9HgNaO2S.

In embodiments, thiomersal is in a concentration of about 0.0005% (w/v) to about 0.1% (w/v), preferably in a concentration of about 0.0005% (w/v) to about 0.05% (w/v).

In embodiments, thiomersal is in a concentration of 0.0005% (w/v), 0.001% (w/v), 0.0015% (w/v), 0.002% (w/v), 0.0025% (w/v), 0.003% (w/v), 0.0035% (w/v), 0.004% (w/v), 0.0045% (w/v), 0.005% (w/v), 0.0055% (w/v), 0.006% (w/v), 0.0065% (w/v), 0.007% (w/v), 0.0075% (w/v), 0.008% (w/v), 0.0085% (w/v), 0.009% (w/v), 0.0095% (w/v), 0.01% (w/v), 0.011% (w/v), 0.012% (w/v), 0.013% (w/v), 0.014% (w/v), 0.015% (w/v), 0.016% (w/v), 0.017% (w/v), 0.018% (w/v), 0.019% (w/v), 0.020% (w/v).

In embodiments, thiomersal is in a concentration of about 0.001% (w/v). In preferred embodiments, thiomersal is in a concentration of about 0.01% (w/v).

In embodiments, the pharmaceutical composition for multidose administration comprises at least one antimicrobial preservative selected from at least one sugar alcohol.

The use of sugar alcohol as antimicrobial preservative is particularly preferred in the context of the invention as sugar alcohols show an as yet undescribed and surprising compatibility with lipid-based carriers encapsulating an RNA.

Sugar alcohols (also called polyhydric alcohols, polyalcohols, alditols or glycitols) are organic compounds, typically derived from sugars, containing one hydroxyl group (—OH) attached to each carbon atom. They are white, water-soluble solids that can occur naturally or be produced industrially by hydrogenation of sugars. Since they contain multiple —OH groups, they are classified as polyols. Common sugar alcohols comprise ethylene glycol, glycerol, erythritol, threitol, arabitol, xylitol, ribitol, mannitol, sorbitol, galactitol, fucitol, iditol, inositol, volemitol, isomalt, maltitol, lactitol, maltotriitol, maltotetraitol, and polyglycitol. Most sugar alcohols are considered to have a certain antimicrobial activity and may therefore be used as suitable sugar alcohol in the context of the invention.

In preferred embodiments, the at least one sugar alcohol of the pharmaceutical composition for multidose administration is selected from xylitol, sorbitol, and/or glycerol, or a combination thereof.

In particularly preferred embodiments, the at least one sugar alcohol is xylitol.

In embodiments, the at least one sugar alcohol (e.g. xylitol) is in a concentration of about 10 mM to about 500 mM, preferably in a concentration of about 10 mM to about 200 mM, more preferably in a concentration of about 25 mM to about 200 mM, even more preferably in a concentration of about 25 mM to about 150 mM

In specific embodiments, the at least one sugar alcohol (e.g. xylitol) is in a concentration of 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, 100 mM, 110 mM, 120 mM, 130 mM, 140 mM, 150 mM, 160 mM, 170 mM, 180 mM, 190 mM, or 200 mM.

In preferred embodiments, the pharmaceutical composition for multidose administration comprising lipid-based carriers encapsulating an RNA may comprise more than one, preferably 2, 3, 4, 5, 6 or more of the antimicrobial preservatives as defined above.

In embodiments, the pharmaceutical composition for multidose administration may comprise more than one aromatic alcohol, preferably selected from benzyl alcohol, phenylethyl alcohol, or phenoxyethanol.

Accordingly, the pharmaceutical composition for multidose administration may comprise benzyl alcohol, phenylethyl alcohol, and phenoxyethanol. Alternatively, the pharmaceutical composition for multidose administration may comprise benzyl alcohol and phenoxyethanol. Alternatively, the pharmaceutical composition for multidose administration may comprise benzyl alcohol and phenylethyl alcohol. Alternatively, the pharmaceutical composition for multidose administration may comprise phenoxyethanol and phenylethyl alcohol.

In embodiments, the pharmaceutical composition for multidose administration may comprise thiomersal and at least one aromatic alcohol, preferably selected from benzyl alcohol, phenylethyl alcohol, or phenoxyethanol.

Accordingly, the pharmaceutical composition for multidose administration may comprise thiomersal and benzyl alcohol. Alternatively, the pharmaceutical composition for multidose administration may comprise thiomersal and phenylethyl alcohol. Alternatively, the pharmaceutical composition for multidose administration may comprise thiomersal and phenoxyethanol. Alternatively, the pharmaceutical composition for multidose administration may comprise thiomersal, benzyl alcohol and phenoxyethanol. Alternatively, the pharmaceutical composition for multidose administration may comprise thiomersal, benzyl alcohol and phenylethyl alcohol. Alternatively, the pharmaceutical composition for multidose administration may comprise thiomersal, phenoxyethanol and phenylethyl alcohol. Alternatively, the pharmaceutical composition for multidose administration may comprise thiomersal, benzyl alcohol, phenylethyl alcohol, and phenoxyethanol.

In embodiments, the pharmaceutical composition for multidose administration may comprise more than one sugar alcohol, preferably selected from xylitol, sorbitol, and/or glycerol.

Accordingly, the pharmaceutical composition for multidose administration may comprise xylitol, sorbitol, and glycerol. Alternatively, the pharmaceutical composition for multidose administration may comprise xylitol and sorbitol. Alternatively, the pharmaceutical composition for multidose administration may comprise xylitol and glycerol. Alternatively, the pharmaceutical composition for multidose administration may comprise glycerol and sorbitol.

In embodiments, the pharmaceutical composition for multidose administration may comprise thiomersal and at least one sugar alcohol, preferably selected from xylitol, sorbitol, and/or glycerol.

Accordingly, the pharmaceutical composition for multidose administration may comprise thiomersal and xylitol. Alternatively, the pharmaceutical composition for multidose administration may comprise thiomersal and sorbitol. Alternatively, the pharmaceutical composition for multidose administration may comprise thiomersal and glycerol. Alternatively, the pharmaceutical composition for multidose administration may comprise thiomersal, xylitol and sorbitol. Alternatively, the pharmaceutical composition for multidose administration may comprise thiomersal, xylitol and glycerol. Alternatively, the pharmaceutical composition for multidose administration may comprise thiomersal, glycerol and sorbitol. Alternatively, the pharmaceutical composition for multidose administration may comprise thiomersal, xylitol, sorbitol, and glycerol.

In particularly preferred embodiments, the pharmaceutical composition for multidose administration comprises at least two antimicrobial preservatives selected from at least one aromatic alcohol and from at least one sugar alcohol.

In preferred embodiments, the at least one aromatic alcohol is selected from benzyl alcohol, phenoxyethanol, phenylethyl alcohol, or a combination thereof, and the at least one sugar alcohol is selected from xylitol, sorbitol, and/or glycerol, or a combination thereof.

Accordingly, the pharmaceutical composition for multidose administration may comprise phenoxyethanol and xylitol.

Alternatively, the pharmaceutical composition for multidose administration may comprise phenoxyethanol and sorbitol.

Alternatively, the pharmaceutical composition for multidose administration may comprise phenoxyethanol and glycerol.

Alternatively, the pharmaceutical composition for multidose administration may comprise benzyl alcohol and xylitol.

Alternatively, the pharmaceutical composition for multidose administration may comprise benzyl alcohol and sorbitol.

Alternatively, the pharmaceutical composition for multidose administration may comprise benzyl alcohol and glycerol.

Alternatively, the pharmaceutical composition for multidose administration may comprise phenylethyl alcohol and xylitol.

Alternatively, the pharmaceutical composition for multidose administration may comprise phenylethyl alcohol and sorbitol.

Alternatively, the pharmaceutical composition for multidose administration may comprise phenylethyl alcohol and glycerol.

In preferred embodiments, the pharmaceutical composition for multidose administration comprises at least two antimicrobial preservatives selected from at least one aromatic alcohol and from at least one sugar alcohol, wherein the at least one aromatic alcohol is phenoxyethanol and the least one sugar alcohol is xylitol.

Accordingly, the pharmaceutical composition for multidose administration may comprise phenoxyethanol and xylitol.

In embodiments, the pharmaceutical composition for multidose administration comprises at least two aromatic alcohols and at least one sugar alcohol.

Accordingly, the pharmaceutical composition for multidose administration may comprise xylitol, benzyl alcohol and phenoxyethanol. Alternatively, the pharmaceutical composition for multidose administration may comprise xylitol, benzyl alcohol and phenylethyl alcohol. Alternatively, the pharmaceutical composition for multidose administration may comprise xylitol, phenoxyethanol and phenylethyl alcohol. Alternatively, the pharmaceutical composition for multidose administration may comprise sorbitol, benzyl alcohol and phenoxyethanol. Alternatively, the pharmaceutical composition for multidose administration may comprise sorbitol, benzyl alcohol and phenylethyl alcohol. Alternatively, the pharmaceutical composition for multidose administration may comprise sorbitol, phenoxyethanol and phenylethyl alcohol. Alternatively, the pharmaceutical composition for multidose administration may comprise glycerol, benzyl alcohol and phenoxyethanol. Alternatively, the pharmaceutical composition for multidose administration may comprise glycerol, benzyl alcohol and phenylethyl alcohol. Alternatively, the pharmaceutical composition for multidose administration may comprise glycerol, phenoxyethanol and phenylethyl alcohol.

In embodiments, the pharmaceutical composition for multidose administration comprises at least three aromatic alcohols and at least one sugar alcohol.

Accordingly, the pharmaceutical composition for multidose administration may comprise xylitol, benzyl alcohol, phenoxyethanol and phenylethyl alcohol. Alternatively, the pharmaceutical composition for multidose administration may comprise sorbitol, benzyl alcohol, phenoxyethanol and phenylethyl alcohol. Alternatively, the pharmaceutical composition for multidose administration may comprise glycerol, benzyl alcohol, phenoxyethanol and phenylethyl alcohol.

In embodiments, the pharmaceutical composition for multidose administration comprises at least one aromatic alcohol and at least two sugar alcohols.

Accordingly, the pharmaceutical composition for multidose administration may comprise phenoxyethanol, xylitol and sorbitol. Alternatively, the pharmaceutical composition for multidose administration may comprise phenoxyethanol, xylitol and glycerol. Alternatively, the pharmaceutical composition for multidose administration may comprise phenoxyethanol, glycerol and sorbitol. Alternatively, the pharmaceutical composition for multidose administration may comprise benzyl alcohol, xylitol and sorbitol. Alternatively, the pharmaceutical composition for multidose administration may comprise benzyl alcohol, xylitol and glycerol. Alternatively, the pharmaceutical composition for multidose administration may comprise benzyl alcohol, glycerol and sorbitol. Alternatively, the pharmaceutical composition for multidose administration may comprise phenylethyl alcohol, xylitol and sorbitol. Alternatively, the pharmaceutical composition for multidose administration may comprise phenylethyl alcohol, xylitol and glycerol. Alternatively, the pharmaceutical composition for multidose administration may comprise phenylethyl alcohol, glycerol and sorbitol.

In embodiments, the pharmaceutical composition for multidose administration comprises at least one aromatic alcohol and at least three sugar alcohols.

Accordingly, the pharmaceutical composition for multidose administration may comprise phenoxyethanol, xylitol, sorbitol, and glycerol. Alternatively, the pharmaceutical composition for multidose administration may comprise benzyl alcohol, xylitol, sorbitol, and glycerol. Alternatively, the pharmaceutical composition for multidose administration may comprise phenylethyl alcohol, xylitol, sorbitol, and glycerol.

In embodiments, the pharmaceutical composition for multidose administration comprises at least two aromatic alcohols and at least two sugar alcohols.

Accordingly, the pharmaceutical composition for multidose administration may comprise phenoxyethanol, phenylethyl alcohol, xylitol, and sorbitol. Alternatively, the pharmaceutical composition for multidose administration may comprise phenoxyethanol, benzyl alcohol, xylitol, and sorbitol. Alternatively, the pharmaceutical composition for multidose administration may comprise phenylethyl alcohol, benzyl alcohol, xylitol, and sorbitol. Alternatively, the pharmaceutical composition for multidose administration may comprise phenoxyethanol, phenylethyl alcohol, xylitol, and glycerol. Alternatively, the pharmaceutical composition for multidose administration may comprise phenoxyethanol, benzyl alcohol, xylitol, and glycerol. Alternatively, the pharmaceutical composition for multidose administration may comprise phenylethyl alcohol, benzyl alcohol, xylitol, and glycerol. Alternatively, the pharmaceutical composition for multidose administration may comprise phenoxyethanol, phenylethyl alcohol, glycerol, and sorbitol. Alternatively, the pharmaceutical composition for multidose administration may comprise phenoxyethanol, benzyl alcohol, glycerol, and sorbitol. Alternatively, the pharmaceutical composition for multidose administration may comprise phenylethyl alcohol, benzyl alcohol, glycerol, and sorbitol.

In embodiments, the pharmaceutical composition for multidose administration comprises at least three aromatic alcohols and at least two sugar alcohols.

Accordingly, the pharmaceutical composition for multidose administration may comprise xylitol, sorbitol, benzyl alcohol, phenoxyethanol and phenylethyl alcohol. Alternatively, the pharmaceutical composition for multidose administration may comprise xylitol, glycerol, benzyl alcohol, phenoxyethanol and phenylethyl alcohol. Alternatively, the pharmaceutical composition for multidose administration may comprise sorbitol, glycerol, benzyl alcohol, phenoxyethanol and phenylethyl alcohol.

In embodiments, the pharmaceutical composition for multidose administration comprises at least two aromatic alcohols and at least three sugar alcohols.

Accordingly, the pharmaceutical composition for multidose administration may comprise xylitol, sorbitol, glycerol, phenoxyethanol and phenylethyl alcohol. Alternatively, the pharmaceutical composition for multidose administration may comprise xylitol, sorbitol, glycerol, benzyl alcohol, phenoxyethanol. Alternatively, the pharmaceutical composition for multidose administration may comprise xylitol, sorbitol, glycerol, benzyl alcohol, phenylethyl alcohol.

In embodiments, the pharmaceutical composition for multidose administration comprises at least three aromatic alcohols and at least three sugar alcohols.

Accordingly, the pharmaceutical composition for multidose administration may comprise xylitol, sorbitol, glycerol, phenoxyethanol, phenylethyl alcohol, and benzyl alcohol.

In embodiments where at least one aromatic alcohol (e.g. phenoxyethanol) and at least one sugar alcohol (e.g. xylitol) are used as antimicrobial preservatives, the at least one aromatic alcohol (e.g. phenoxyethanol) is in a concentration of about 0.1% (w/v) to about 5% (w/v), preferably in a concentration of about 0.1% (w/v) to about 2%, and the at least one sugar alcohol (e.g. xylitol) is in a concentration of about 10 mM to about 500 mM, preferably in a concentration of about 10 mM to about 200 mM, more preferably in a concentration of about 25 mM to about 200 mM, even more preferably in a concentration of about 25 mM to about 150 mM In preferred embodiments of the first aspect, the pharmaceutical composition for multidose administration comprises phenoxyethanol and xylitol, wherein phenoxyethanol is in a concentration of about 0.1% (w/v) to about 2%, preferably 0.5% (w/v), and wherein xylitol is in a concentration of about 10 mM to about 200 mM, preferably about 25 mM to about 150 mM.

In embodiments, the concentration of the RNA in the pharmaceutical composition for multidose administration is in a range of about 100 μg/ml to about 1 mg/ml.

In embodiments, the concentration of the RNA in the pharmaceutical composition for multidose administration is for example about 100 μg/ml, about 200 μg/ml, about 300 μg/ml, about 400 μg/ml, about 500 μg/ml, about 600 μg/ml, about 700 μg/ml, about 800 μg/ml, about 900 μg/ml, about 1 mg/ml.

In various embodiments, the RNA of the pharmaceutical composition for multidose administration has a certain RNA integrity.

The term “RNA integrity” generally describes whether the complete RNA sequence is present in the pharmaceutical composition. Low RNA integrity could be due to, amongst others, RNA degradation, RNA cleavage, incorrect or incomplete chemical synthesis of the RNA, incorrect base pairing, integration of modified nucleotides or the modification of already integrated nucleotides, lack of capping or incomplete capping, lack of polyadenylation or incomplete polyadenylation, or incomplete RNA in vitro transcription. RNA is a fragile molecule that can easily degrade, which may be caused e.g. by temperature, ribonucleases, pH or other factors (e.g. nucleophilic attacks, hydrolysis etc.), which may reduce the RNA integrity and, consequently, the functionality of the RNA.

The skilled person can choose from a variety of different chromatographic or electrophoretic methods for determining an RNA integrity. Chromatographic and electrophoretic methods are well-known in the art. In case chromatography is used (e.g. RP-HPLC), the analysis of the integrity of the RNA may be based on determining the peak area (or “area under the peak”) of the full length RNA in a corresponding chromatogram. The peak area may be determined by any suitable software which evaluates the signals of the detector system. The process of determining the peak area is also referred to as integration. The peak area representing the full length RNA is typically set in relation to the peak area of the total RNA in a respective sample. The RNA integrity may be expressed in % RNA integrity.

In the context of the invention, RNA integrity may be determined using analytical (RP)HPLC. Typically, a test sample of the pharmaceutical composition for multidose administration comprising lipid based carrier encapsulating RNA may be treated with a detergent (e.g. about 2% Triton X100) to dissociate the lipid based carrier and to release the encapsulated RNA. The released RNA may be captured using suitable binding compounds, e.g. Agencourt AMPure XP beads (Beckman Coulter, Brea, CA, USA) essentially according to the manufacturer's instructions. Following preparation of the RNA sample, analytical (RP)HPLC may be performed to determine the integrity of RNA. Typically, for determining RNA integrity, the RNA samples may be diluted to a concentration of 0.1 g/l using e.g. water for injection (WFI). About 10 μl of the diluted RNA sample may be injected into an HPLC column (e.g. a monolithic poly(styrene-divinylbenzene) matrix). Analytical (RP)HPLC may be performed using standard conditions, for example: Gradient 1: Buffer A (0.1 M TEAA (pH 7.0)); Buffer B (0.1 M TEAA (pH 7.0) containing 25% acetonitrile). Starting at 30% buffer B the gradient extended to 32% buffer B in 2 min, followed by an extension to 55% buffer B over 15 minutes at a flow rate of 1 ml/min. HPLC chromatograms are typically recorded at a wavelength of 260 nm. The obtained chromatograms may be evaluated using a software and the relative peak area may be determined in percent (%) as commonly known in the art. The relative peak area indicates the amount of RNA that has 100% RNA integrity. Since the amount of the RNA injected into the HPLC is typically known, the analysis of the relative peak area provides information on the integrity of the RNA. Thus, if e.g. 100 ng RNA have been injected in total, and 100 ng are determined as the relative peak area, the RNA integrity would be 100%. If, for example, the relative peak area would correspond to 80 ng, the RNA integrity would be 80%. Accordingly, RNA integrity in the context of the invention is determined using analytical HPLC, preferably analytical RP-HPLC.

In embodiments, the RNA has an RNA integrity ranging from about 40% to about 100%. In embodiments, the RNA has an RNA integrity ranging from about 50% to about 100%. In embodiments, the RNA has an RNA integrity ranging from about 60% to about 100%. In embodiments, the RNA has an RNA integrity ranging from about 70% to about 100%. In embodiments, the RNA integrity is for example about 50%, about 60%, about 70%, about 80%, or about 90%. RNA is suitably determined using analytical HPLC, preferably analytical RP-HPLC.

In preferred embodiments, the RNA has an RNA integrity of at least about 50%, preferably of at least about 60%, more preferably of at least about 70%, most preferably of at least about 80%. RNA is suitably determined using analytical HPLC, preferably analytical RP-HPLC.

In various embodiments, the RNA of the pharmaceutical composition for multidose administration does not exceed a certain proportion of free RNA.

The term “free RNA” or “non-complexed RNA” or “non-encapsulated RNA” comprise the RNA molecules that are not encapsulated in the lipid-based carriers as defined herein. During formulation of the pharmaceutical composition (e.g. during encapsulation of the RNA into the lipid-based carriers and after adding the antimicrobial preservative), free RNA may represent a contamination or an impurity. A large proportion of non-encapsulated or free RNA may also be an indicator for destabilization of a the lipid-based carriers of the composition (e.g. caused by an added antimicrobial preservative).

The skilled person can choose from a variety of different methods for determining the amount and/or the proportion of free RNA in the pharmaceutical composition for multidose administration. Free RNA in the pharmaceutical composition for multidose administration may be determined by chromatographic methods (e.g. AEX, SEC) or by using probes (e.g. dyes) that bind to free RNA in the composition. In the context of the invention, the amount of free RNA or non-encapsulated RNA may be determined using a dye based assay. Suitable dyes that may be used to determine the amount and/or the proportion of free RNA comprise RiboGreen®, PicoGreen® dye, OliGreen® dye, QuantiFluor® RNA dye, Qubit® RNA dye, Quant-iT™ RNA dye, TOTO®-1 dye, YOYO®-1 dye. Such dyes are suitable to discriminate between free RNA and encapsulated RNA. Reference standards consisting of defined amounts of free RNA or encapsulated RNA may be used and mixed with the respective reagent (e.g. RiboGreen® reagent (Excitation 500 nm/Emission 525 nm)) as recommended by the supplier's instructions. Typically, the free RNA of the pharmaceutical composition for multidose administration is quantitated using the Quant-iT RiboGreen® RNA Reagent according to the manufacturer's instructions. The proportion of free RNA in the context of the invention is typically determined using a RiboGreen assay.

In embodiments, the pharmaceutical composition for multidose administration comprises free RNA ranging from about 30% to about 0%. In embodiments, the pharmaceutical composition for multidose administration comprises about 20% free RNA (and about 80% encapsulated RNA), about 15% free RNA (and about 85% encapsulated RNA), about 10% free RNA (and about 90% encapsulated RNA), or about 5% free RNA (and about 95% encapsulated RNA). In preferred embodiments, the pharmaceutical composition for multidose administration comprises less than about 20% free RNA, preferably less than about 15% free RNA, more preferably less than about 10% free RNA, most preferably less than about 5% free RNA.

The term “encapsulated RNA” comprise the RNA molecules that are encapsulated in the lipid-based carriers as defined herein. The proportion of encapsulated RNA in the context of the invention is typically determined using a RiboGreen assay.

Accordingly, in embodiments, about 70% to about 100% of the RNA in the pharmaceutical composition for multidose administration is encapsulated in the lipid-based carriers. In embodiments, the pharmaceutical composition for multidose administration comprises about 80% encapsulated RNA (and about 20% free RNA), about 85% encapsulated RNA (and about 15% free RNA), about 90% encapsulated RNA (and about 10% free RNA), or about 95% encapsulated RNA (and 5% about free RNA).

In preferred embodiments, 80% of the RNA comprised in the pharmaceutical composition for multidose administration is encapsulated, preferably 85% of the RNA comprised in the composition is encapsulated, more preferably 90% of the RNA comprised in the composition is encapsulated, most preferably 95% of the RNA comprised in the composition is encapsulated.

In various embodiments, the pharmaceutical composition for multidose administration comprises purified RNA. It may be suitably to apply certain purification steps during RNA production to achieve certain RNA purity levels in regards of various impurities. Accordingly, the RNA used for formulation of the lipid-based carriers has been purified (before formulation/encapsulation) to remove various RNA impurities.

In embodiments, the RNA of the pharmaceutical composition for multidose administration (the RNA that is encapsulated in the lipid based carriers) is a purified RNA. Accordingly, in embodiments, the RNA encapsulated in lipid based carriers has been purified prior to formulation of the lipid based carriers encapsulating the RNA.

The term “purified RNA” or “purified mRNA” as used herein has to be understood as RNA which has a higher purity after certain purification steps (e.g. HPLC, TFF, Oligo d(T) purification, precipitation, filtration, AEX) than the starting material (e.g. in vitro transcribed RNA). Typical impurities that are essentially not present in purified RNA comprise peptides or proteins (e.g. enzymes derived from RNA in vitro transcription, e.g. RNA polymerases, RNases, pyrophosphatase, restriction endonuclease, DNase), spermidine, BSA, short abortive RNA sequences, RNA fragments (short double stranded RNA fragments, short single stranded RNA fragments, abortive RNA sequences etc.), free nucleotides (modified nucleotides, conventional NTPs, cap analogue), template DNA fragments, buffer components (HEPES, TRIS, MgCl2, CaCl2) etc. Other potential impurities may be derived from e.g. fermentation procedures and comprise bacterial impurities (bioburden, bacterial DNA, bacterial RNA) or impurities derived from purification procedures (organic solvents etc.). Accordingly, it is desirable in this regard for the “degree of RNA purity” to be as close as possible to 100%.

Accordingly, “purified RNA” as used herein has a degree of purity of more than 75%, 80%, 85%, very particularly 90%, 91%, 92%, 93%, 94%, 95%, 96%, 97%, 98% and most favourably 99% or more. The degree of purity may for example be determined by an analytical HPLC, wherein the percentages provided above correspond to the ratio between the area of the peak for the target RNA and the total area of all peaks representing all the by-products. Alternatively, the degree of purity may for example be determined by an analytical agarose gel electrophoresis or capillary gel electrophoresis.

In embodiments, the RNA of the pharmaceutical composition for multidose administration is an RP-HPLC purified RNA and/or a tangential flow filtration (TFF) purified RNA.

In preferred embodiments, the RNA of the pharmaceutical composition for multidose administration (the RNA that is encapsulated in the lipid based carriers) is an RP-HPLC purified RNA, wherein the RNA has been purified using a method as described in published patent application WO2008/077592, the specific disclosure relating to the published PCT claims 1 to 28 herewith incorporated by reference.

In embodiments, the RNA of the pharmaceutical composition for multidose administration (the RNA that is encapsulated in the lipid based carriers) has been purified by at least one step of TFF against a salt buffer, preferably against an NaCl buffer. In preferred embodiments, a tangential flow filtration method as described in published patent application WO2016/193206 may be used, the specific disclosure relating to the published PCT claims 1 to 48 herewith incorporated by reference.

In preferred embodiments, the RNA of pharmaceutical composition for multidose administration is an artificial RNA.

The term “artificial RNA” as used herein is intended to refer to an RNA that does not occur naturally. In other words, an artificial RNA may be understood as a non-natural RNA molecule. Such RNA molecules may be non-natural due to its individual sequence (e.g. G/C content modified coding sequence, heterologous UTRs) and/or due to other modifications, e.g. structural modifications of nucleotides. Typically, artificial RNA may be designed and/or generated by genetic engineering to correspond to a desired artificial sequence of nucleotides. In this context, an artificial RNA is a sequence that may not occur naturally, i.e. a sequence that differs from the wild type sequence/the naturally occurring sequence by at least one nucleotide (via e.g. codon modification as further specified below). The term “artificial RNA” is not restricted to mean “one single molecule” but is understood to comprise an ensemble of essentially identical RNA molecules. Accordingly, the term may relate to a plurality of essentially identical RNA molecules.

In embodiments, the RNA of pharmaceutical composition for multidose administration does comprise (chemically) modified nucleotides.

In the context of the invention, the terms “modified nucleotides” or “chemically modified nucleotides” do not encompass 5′ cap structures (e.g. cap0, cap1 as defined herein). Additionally, the term “modified nucleotides” does not relate to modifications of the codon usage of e.g. a respective coding sequence. The terms “modified nucleotides” or “chemically modified nucleotides” do encompass all potential natural and non-natural chemical modifications of the building blocks of an RNA, namely the ribonucleotides A, G, C, U.

A chemically modified RNA may comprise nucleotide analogues/modifications, e.g. backbone modifications, sugar modifications or base modifications. A backbone modification is a chemical modification in which phosphates of the backbone of the nucleotides of the RNA are modified. A sugar modification is a chemical modification of the sugar of the nucleotides of the RNA. Furthermore, a base modification is a chemical modification of the base moiety of the nucleotides of the RNA. Examples of chemically modified nucleotides comprise 2-amino-6-chloropurineriboside-5′-triphosphate, 2-Aminopurine-riboside-5′-triphosphate; 2-aminoadenosine-5′-triphosphate, 2′-Amino-2′-deoxycytidine-triphosphate, 2-thiocytidine-5′-triphosphate, 2-thiouridine-5′-triphosphate, 2′-Fluorothymidine-5′-triphosphate, 2′-O-Methyl-inosine-5′-triphosphate 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-Bromo-2′-deoxycytidine-5′-triphosphate, 5-Bromo-2′-deoxyuridine-5′-triphosphate, 5-iodocytidine-5′-triphosphate, 5-lodo-2′-deoxycytidine-5′-triphosphate, 5-iodouridine-5′-triphosphate, 5-lodo-2′-deoxyuridine-5′-triphosphate, 5-methylcytidine-5′-triphosphate, 5-methyluridine-5′-triphosphate, 5-Propynyl-2′-deoxycytidine-5′-triphosphate, 5-Propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, O6 -methylguanosine-5′-triphosphate, pseudouridine-5′-triphosphate, or puromycin-5′-triphosphate, xanthosine-5′-triphosphate. Particular preference is given to nucleotides for base modifications selected from the group of base-modified nucleotides consisting of 5-methylcytidine-5′-triphosphate, 7-deazaguanosine-5′-triphosphate, 5-bromocytidine-5′-triphosphate, and pseudouridine-5′-triphosphate, pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine, 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, 5′-O-(1-thiophosphate)-adenosine, 5′-O-(1-thiophosphate)-cytidine, 5′-O-(1-thiophosphate)-guanosine, 5′-O-(1-thiophosphate)-uridine, 5′-O-(1-thiophosphate)-pseudouridine, 6-aza-cytidine, 2-thio-cytidine, alpha-thio-cytidine, Pseudo-iso-cytidine, 5-aminoallyl-uridine, 5-iodo-uridine, N1-methyl-pseudouridine, 5,6-dihydrouridine, alpha-thio-uridine, 4-thio-uridine, 6-aza-uridine, 5-hydroxy-uridine, deoxy-thymidine, 5-methyl-uridine, Pyrrolo-cytidine, inosine, alpha-thio-guanosine, 6-methyl-guanosine, 5-methyl-cytdine, 8-oxo-guanosine, 7-deaza-guanosine, N1-methyl-adenosine, 2-amino-6-Chloro-purine, N6-methyl-2-amino-purine, Pseudo-iso-cytidine, 6-Chloro-purine, N6-methyl-adenosine, alpha-thio-adenosine, 8-azido-adenosine, 7-deaza-adenosine.

In embodiments, the RNA of pharmaceutical composition for multidose administration comprises chemically modified nucleotides selected from pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine. In preferred embodiments, the RNA of the composition comprises pseudouridine (ψ) or N1-methylpseudouridine (m1Φ), 5-methylcytosine, and 5-methoxyuridine, suitably ψ or m1ψ.

In embodiments, essentially all, e.g. essentially 100% of the uracil in the coding sequence (or the full length sequence) of the RNA have a chemical modification, preferably a chemical modification is in the 5-position of the uracil (e.g. ψ or m1ψ).

Incorporating modified nucleotides such as e.g. pseudouridine (ψ), N1-methylpseudouridine (m1ψ), 5-methylcytosine, and/or 5-methoxyuridine into the coding sequence may be advantageous as unwanted innate immune responses (upon administration of the composition) may be adjusted or reduced (if required).

In embodiments, the RNA of the pharmaceutical composition for multidose administration does not comprise (chemically) modified nucleotides as defined herein. In preferred embodiments, the RNA of the composition does not comprise pseudouridine (ψ), N1-methylpseudouridine (m1ψ), 5-methylcytosine, and 5-methoxyuridine.

In embodiments, the RNA of the pharmaceutical composition for multidose administration is an in vitro transcribed RNA, preferably wherein RNA in vitro transcription has been performed in the presence of a sequence optimized mixture of nucleotides and, optionally, in the presence of a cap analog (e.g. a cap1 analog).

In embodiments, the RNA of the pharmaceutical composition for multidose administration has a length ranging from about 50 nucleotides to about 20000 nucleotides, about 200 nucleotides to about 10000 nucleotides, about 500 nucleotides to about 10000 nucleotides, or about 1000 nucleotides to about 10000 nucleotides, or preferably about 1000 nucleotides to about 5000 nucleotides, or even more preferably about 2000 to about 5000 nucleotides. In embodiments, the RNA is at least 500 nt in length, the RNA is at least 500 nt in length, the RNA is at least 1000 nt in length, the RNA is at least 2000 nt in length, the RNA is at least 3000 nt in length, the RNA is at least 4000 nt in length, or the RNA is at least 5000 nt in length.

In various embodiments, the RNA of the pharmaceutical composition for multidose administration is a therapeutic RNA.

The term “therapeutic RNA” relates to an RNA providing a therapeutic modality. The term “therapeutic” in that context has to be understood as “providing a therapeutic function” or as “being suitable for therapy or administration”. However, “therapeutic” in that context should not at all to be understood as being limited to a certain therapeutic modality. Examples for therapeutic modalities may be the provision of a coding sequence (via said therapeutic RNA) that encodes for a peptide or protein (wherein said peptide or protein has a certain therapeutic function, e.g. an antigen for a vaccine, or an enzyme for protein replacement therapies). A further therapeutic modality may be genetic engineering, wherein the RNA provides or orchestrates factors to e.g. manipulate DNA and/or RNA in a cell or a subject. Typically, the term “therapeutic RNA” does not include natural RNA extracts or RNA preparations (e.g. obtained from bacteria, or obtained from plants) that are not suitable for administration to a subject (e.g. animal, human). For being suitable for a therapeutic purpose, the RNA of the invention may be an artificial RNA.

In embodiments, the RNA of the pharmaceutical composition for multidose administration, preferably the therapeutic RNA, is selected from viral RNA, retroviral RNA, replicon RNA, small interfering RNA (siRNA), antisense RNA, saRNA (small activating RNA), CRISPR RNA (small guide RNA, sgRNA), ribozymes, aptamers, riboswitches, immunostimulating RNA, transfer RNA (tRNA), ribosomal RNA (rRNA), small nuclear RNA (snRNA), small nucleolar RNA (snoRNA), microRNA (miRNA), Piwi-interacting RNA (piRNA), self-replicating RNA, circular RNA, or mRNA.

In embodiments, the RNA of the pharmaceutical composition for multidose administration, preferably the therapeutic RNA, is a non-coding RNA, preferably a CRISPR/Cas9 guide RNA or a small interfering RNA (siRNA).

As used herein, the term “guide RNA” (gRNA) relates to an RNA molecule capable of targeting a CRISPR-associated protein or a CRISPR-associated endonuclease to a target DNA sequence of interest. The term guide RNA has to be understood in its broadest sense, and may comprise two-molecule gRNAs (“tracrRNA/crRNA”) comprising crRNA (“CRISPR RNA” or “targeter-RNA” or “crRNA” or “crRNA repeat”) and a corresponding tracrRNA (“trans-acting CRISPR RNA” or “activator-RNA” or “tracrRNA”) molecule, or single-molecule gRNAs. A “sgRNA” typically comprises a crRNA connected at its 3′ end to the 5′ end of a tracrRNA through a “loop” sequence.

In preferred embodiments, the RNA of the pharmaceutical composition for multidose administration is a coding RNA. Most preferably, said coding RNA may be selected from an mRNA, a (coding) self-replicating RNA, a (coding) circular RNA, a (coding) viral RNA, or a (coding) replicon RNA.

A coding RNA can be any type of RNA construct (for example a double stranded RNA, a single stranded RNA, a circular double stranded RNA, or a circular single stranded RNA) characterized in that said coding RNA comprises at least one coding sequence (cds) that is translated into at least one amino-acid sequence (e.g. upon administration to e.g a cell).

In embodiments, the RNA of the pharmaceutical composition for multidose administration is a circular RNA. As used herein, the terms “circular RNA” or “circRNAs” have to be understood as a circular polynucleotide constructs that may encode at least one peptide or protein. Preferably, such a circRNA is a single stranded RNA molecule. In preferred embodiments, said circRNA comprises at least one coding sequence encoding at least one peptide or protein as defined herein, or a fragment or variant thereof.

In embodiments, the RNA of the pharmaceutical composition for multidose administration is a replicon RNA. The term “replicon RNA” is e.g. intended to be an optimized self-replicating RNA. Such constructs may include replicase elements derived from e.g. alphaviruses (e.g. SFV, SIN, VEE, or RRV) and the substitution of the structural virus proteins with the nucleic acid of interest (that is, the coding sequence encoding an antigenic peptide or protein as defined herein).

Alternatively, the replicase may be provided on an independent coding RNA construct or a coding DNA construct. Downstream of the replicase may be a sub-genomic promoter that controls replication of the replicon RNA.

In preferred embodiments, the RNA of the pharmaceutical composition for multidose administration is not a replicon RNA.

In preferred embodiments, the RNA of the pharmaceutical composition for multidose administration is not a self-replicating RNA.

In particularly preferred embodiments, the RNA of the pharmaceutical composition for multidose administration is an mRNA.

In preferred embodiments, the RNA of the pharmaceutical composition for multidose administration comprises at least one coding sequence.

In embodiments, the length the coding sequence (which may be a part of the RNA) may be at least or greater than about 50, 60, 70, 80, 90, 100, 150, 200, 250, 300, 350, 400, 450, 500, 600, 700, 800, 900, 1000, 1200, 1400, 1600, 1800, 2000, 2500, 3000, 3500, 4000, 5000, or 6000 nucleotides. In embodiments, the length of the coding sequence may be in a range of from about 300 to about 2000 nucleotides.

In preferred embodiments, the RNA of the pharmaceutical composition for multidose administration comprises at least one codon modified coding sequence.

In preferred embodiments, the at least one coding sequence of the RNA is a codon modified coding sequence. Suitably, the amino acid sequence encoded by the at least one codon modified coding sequence is not being modified compared to the amino acid sequence encoded by the corresponding wild type coding sequence.

The term “codon modified coding sequence” relates to coding sequences that differ in at least one codon (triplets of nucleotides coding for one amino acid) compared to the corresponding wild type coding sequence. Suitably, a codon modified coding sequence in the context of the invention may show improved resistance to in vivo degradation and/or improved stability in vivo, and/or improved translatability in vivo and/or improved temperature stability upon storage. Codon modifications in the broadest sense make use of the degeneracy of the genetic code wherein multiple codons may encode the same amino acid and may be used interchangeably to optimize/modify the coding sequence for in vivo applications as outlined above.

In preferred embodiments, the at least one coding sequence of the RNA is a codon modified coding sequence, wherein the codon modified coding sequence is selected from C maximized coding sequence, CAI maximized coding sequence, human codon usage adapted coding sequence, G/C content modified coding sequence, and G/C optimized coding sequence, or any combination thereof.

In embodiments, the RNA of the pharmaceutical composition for multidose administration may be codon modified, wherein the C content of the at least one coding sequence may be increased, preferably maximized, compared to the C content of the corresponding wild type coding sequence (herein referred to as “C maximized coding sequence”). The amino acid sequence encoded by the C maximized coding sequence of the nucleic acid is preferably not modified compared to the amino acid sequence encoded by the respective wild type coding sequence. The generation of a C maximized RNA sequences be carried out using a modification method according to WO2015/062738. In this context, the disclosure of WO2015/062738 is included herewith by reference.

In embodiments, the RNA of the pharmaceutical composition for multidose administration may be codon modified, wherein the codons in the at least one coding sequence may be adapted to human codon usage (herein referred to as “human codon usage adapted coding sequence”). Codons encoding the same amino acid occur at different frequencies in humans. Accordingly, the coding sequence of the RNA is preferably modified such that the frequency of the codons encoding the same amino acid corresponds to the naturally occurring frequency of that codon according to the human codon usage. Such a procedure may be applied for each amino acid encoded by the coding sequence of the RNA to obtain sequences adapted to human codon usage.

In embodiments, the RNA of the pharmaceutical composition for multidose administration may be codon modified, wherein the codon adaptation index (CAI) may be increased or preferably maximised in the at least one coding sequence (herein referred to as “CAI maximized coding sequence”). It is preferred that all codons of the wild type sequence that are relatively rare in e.g. a human are exchanged for a respective codon that is frequent in the e.g. a human, wherein the frequent codon encodes the same amino acid as the relatively rare codon. Suitably, the most frequent codons are used for each amino acid of the encoded protein. Suitably, the RNA may comprise at least one coding sequence, wherein the codon adaptation index (CAI) of the at least one coding sequence is at least 0.5, at least 0.8, at least 0.9 or at least 0.95. Most preferably, the codon adaptation index (CAI) of the at least one coding sequence is 1 (CAI=1). Such a procedure (as exemplified for Ala) may be applied for each amino acid encoded by the coding sequence of the nucleic acid to obtain CAI maximized coding sequences.

In preferred embodiments, the RNA of the pharmaceutical composition for multidose administration may be codon modified, wherein the G/C content of the at least one coding sequence may be optimized compared to the G/C content of the corresponding wild type coding sequence (herein referred to as “G/C content optimized coding sequence”). “Optimized” in that context refers to a coding sequence wherein the G/C content is preferably increased to the essentially highest possible G/C content. The amino acid sequence encoded by the G/C content optimized coding sequence of the RNA is preferably not modified as compared to the amino acid sequence encoded by the respective wild type coding sequence. The generation of a G/C content optimized RNA sequences may be carried out using a method according to WO2002/098443. In this context, the disclosure of WO2002/098443 is included in its full scope in the present invention.

In preferred embodiments, the RNA of the pharmaceutical composition for multidose administration may be codon modified, wherein the G/C content of the at least one coding sequence may be modified compared to the G/C content of the corresponding wild type coding sequence (herein referred to as “G/C content modified coding sequence”). In this context, the terms “G/C optimization” or “G/C content modification” relate to an RNA that comprises a modified, preferably an increased number of guanosine and/or cytosine nucleotides as compared to the corresponding wild type coding sequence. Such an increased number may be generated by substitution of codons containing adenosine or thymidine nucleotides by codons containing guanosine or cytosine nucleotides. Advantageously, RNA sequences having an increased G/C content may be more stable or may show a better expression than sequences having an increased A/U. The amino acid sequence encoded by the G/C content modified coding sequence of the RNA is preferably not modified as compared to the amino acid sequence encoded by the respective wild type sequence.

Suitably, the G/C content of the coding sequence of the RNA of the pharmaceutical composition for multidose administration is increased by at least 10%, 20%, 30%, preferably by at least 40% compared to the G/C content of the corresponding wild type coding sequence.

According to various embodiments, the pharmaceutical composition for multidose administration comprises RNA with an increased GC content. Accordingly, the RNA used for encapsulation in the lipid-based carriers may have a certain (increased) GC content.

In various embodiments, the RNA of the pharmaceutical composition for multidose administration has a GC content of about 50% to about 80%. In preferred embodiments, the RNA of the pharmaceutical composition for multidose administration has a GC content of at least about 50%, preferably at least about 55%, more preferably of at least about 60%. In specific embodiments, the RNA of the pharmaceutical composition for multidose administration has a GC content of about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, or about 70%.

In various embodiments, the coding sequence of the RNA has a GC content of about 60% to about 90%. In preferred embodiments, the coding sequence of the RNA has a GC content of at least about 60%, preferably at least about 65%, more preferably of at least about 70%. In specific embodiments, the RNA of the composition has a GC content of about 60%, about 61%, about 62%, about 63%, about 64%, about 65%, about 66%, about 67%, about 68%, about 69%, about 70%, about 710%, about 72%, about 73%, about 74%, about 75%, about 76%, about 77%, about 78%, about 79%, or about 80%.

In various embodiments, the RNA of the pharmaceutical composition for multidose administration composition comprises a 5′-cap structure, preferably a cap1 structure.

Accordingly, in preferred embodiments, the RNA of the pharmaceutical composition for multidose administration comprises a 5′-cap structure, preferably m7G, cap0, cap1, cap2, a modified cap0 or a modified cap1 structure.

The term “5′-cap structure” as used herein is intended to refer to the 5′ structure of the RNA, particularly a guanine nucleotide, positioned at the 5′-end of an RNA, e.g. an mRNA. Preferably, the 5′-cap structure is connected via a 5′-5′-triphosphate linkage to the RNA. Notably, a “5′-cap structure” or a “cap analogue” is not considered to be a “modified nucleotide” or “chemically modified nucleotides” in the context of the invention. 5′-cap structures which may be suitable in the context of the present invention are cap0 (methylation of the first nucleobase, e.g. m7GpppN), cap1 (additional methylation of the ribose of the adjacent nucleotide of m7GpppN), cap2 (additional methylation of the ribose of the 2nd nucleotide downstream of the m7GpppN), cap3 (additional methylation of the ribose of the 3rd nucleotide downstream of the m7GpppN), cap4 (additional methylation of the ribose of the 4th nucleotide downstream of the m7GpppN), ARCA (anti-reverse cap analogue), modARCA (e.g. phosphothioate modARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

A 5-cap (cap0 or cap1) structure may be formed in chemical RNA synthesis, using capping enzymes, or in RNA in vitro transcription (co-transcriptional capping) using cap analogs.

The term “cap analog” as used herein is intended to refer to a non-polymerizable di-nucleotide or tri-nucleotide that has cap functionality in that it facilitates translation or localization, and/or prevents degradation the RNA when incorporated at the 5′-end of the RNA. Non-polymerizable means that the cap analogue will be incorporated only at the 5′-terminus because it does not have a 5′ triphosphate and therefore cannot be extended in the 3′-direction by a template-dependent polymerase, (e.g. a DNA-dependent RNA polymerase). Examples of cap analogues include m7GpppG, m7GpppA, m7GpppoC unmethylated cap analogues (e.g. GpppG); dimethylated cap analogue (e.g. m2,7GpppG), trimethylated cap analogue (e.g. m2,2,7GpppG), dimethylated symmetrical cap analogues (e.g. m7Gpppm7G), or anti reverse cap analogues (e.g. ARCA; m7,2′OmeGpppG, m7,2′dGpppG, m7,3′OmeGpppG, m7,3′dGpppG and their tetraphosphate derivatives). Further cap analogues have been described previously (WO2008/016473, WO2008/157688, WO2009/149253, WO2011/015347, and WO2013/059475). Further suitable cap analogues in that context are described in WO2017/066793, WO2017/066781, WO2017/066791, WO2017/066789, WO2017/053297, WO2017/066782, WO2018/075827 and WO2017/066797 wherein the disclosures relating to cap analogues are incorporated herewith by reference.

In embodiments, a cap1 structure is generated using tri-nucleotide cap analogue as disclosed in WO2017/053297, WO2017/066793, WO2017/066781, WO2017/066791, WO2017/066789, WO2017/066782, WO2018/075827 and WO2017/066797. In particular, any cap analog derivable from the structure disclosed in claim 1-5 of WO2017/053297 may be suitably used to co-transcriptionally generate a cap1 structure. Further, any cap analog derivable from the structure defined in claim 1 or claim 21 of WO2018/075827 may be suitably used to co-transcriptionally generate a cap1 structure.

In preferred embodiments, the RNA of the pharmaceutical composition for multidose administration comprises a cap1 structure.

In preferred embodiments, the cap1 structure of the RNA is formed using co-transcriptional capping using tri-nucleotide cap analog m7G(5′)ppp(5′)(2′OMeA)pG or m7G(5′)ppp(5′)(2′OMeG)pG. A preferred cap1 analog in that context is m7G(5′)ppp(5′)(2′OMeA)pG.

In embodiments, about 70%, 75%, 80%, 85%, 90%, 95% of the RNA of the pharmaceutical composition for multidose administration comprises a cap structure, preferably a cap1 structure, as determined using a capping assay. In preferred embodiments, less than about 20%, 15%, 10%, 5%, 4%, 3%, 2%, 1% of the RNA (species) does not comprises a cap structure as determined using a capping assay.

In preferred embodiments, at least 70%, 80%, or 90% of the RNA of the pharmaceutical composition for multidose administration comprise a cap1 structure.

For determining the presence/absence of a cap0 or a cap1 structure, a capping assays as described in published PCT application WO2015/101416, in particular, as described in claims 27 to 46 of published PCT application WO2015/101416 may be used. Other capping assays that may be used to determine the presence/absence of a cap0 or a cap1 structure of an RNA are described in WO2020/127959, or published PCT applications WO2014/152673 and WO2014/152659.

In preferred embodiments, the RNA of the pharmaceutical composition for multidose administration comprises an m7G(5′)ppp(5′)(2′OMeA) cap structure. In such embodiments, the RNA comprises a 5-terminal m7G cap, and an additional methylation of the ribose of the adjacent nucleotide of m7GpppN, in that case, a 2′O methylated Adenosine. Preferably, about 70%, 75%, 80%, 85%, 90%, 95% of the RNA (species) comprises such a cap1 structure as determined using a capping assay. Preferably, about 95% of the RNA (species) comprises a cap1 structure in the correct orientation (and less that about 5% in reverse orientation) as determined using a capping assay.

In other preferred embodiments, the RNA of the pharmaceutical composition for multidose administration comprises an m7G(5′)ppp(5′)(2′OMeG) cap structure. In such embodiments, the RNA comprises a 5-terminal m7G cap, and an additional methylation of the ribose of the adjacent nucleotide, in that case, a 2′O methylated guanosine. Preferably, about 70%, 75%, 80%, 85%, 90%, 95% of the coding RNA (species) comprises such a cap1 structure as determined using a capping assay.

Accordingly, the first nucleotide of said RNA or mRNA sequence, that is, the nucleotide downstream of the m7G(5′)ppp structure, may be a 2′O methylated guanosine or a 2′O methylated adenosine.

In preferred embodiments, the RNA of the pharmaceutical composition for multidose administration comprises at least one poly(A) sequence, and/or at least one poly(C) sequence, and/or at least one histone stem-loop and/or at least one 5′-UTR and/or at least one 3′-UTR.

In preferred embodiments, the RNA of the pharmaceutical composition for multidose administration comprises at least one heterologous untranslated region (UTR).

The term “untranslated region” or “UTR” or “UTR element” are intended to refer to a part of an RNA typically located 5′ or 3′ of a coding sequence. An UTR is not translated into protein. An UTR may comprise elements for controlling gene expression, also called regulatory elements. Such regulatory elements may be, e.g., ribosomal binding sites, miRNA binding sites, promotor elements etc.

UTRs may harbor regulatory sequence elements that determine RNA turnover, stability, and localization. Moreover, UTRs may harbor sequence elements that enhance translation. In medical application of and RNA, translation into at least one peptide or protein may be of paramount importance to therapeutic efficacy. Certain combinations of 3′-UTRs and/or 5′-UTRs may enhance the expression of operably linked coding sequences encoding peptides or proteins of the invention.

RNA harboring said UTR combinations advantageously enable rapid and transient expression of antigenic peptides or proteins after administration to a subject.

In embodiments, the RNA comprises at least one 5′-UTR, preferably a heterologous 5′-UTR and/or at least one 3′-UTR, preferably a heterologous 3′-UTR.

Heterologous 5′-UTRs or 3′-UTRs may be derived from naturally occurring genes or may be synthetically engineered. In preferred embodiments, the RNA comprises at least one coding sequence as defined herein operably linked to at least one (heterologous) 3′-UTR and/or at least one (heterologous) 5′-UTR.

In preferred embodiments, the RNA of the pharmaceutical composition for multidose administration comprises at least one heterologous 3′-UTR.

The term “3′-untranslated region” or “3′-UTR” or “3′-UTR element” are intended to refer to a part of an RNA molecule located 3′ (i.e. downstream) of a coding sequence and which is not translated into protein. A 3′-UTR may be part of an RNA, located between a coding sequence and an optional terminal poly(A) sequence. A 3′-UTR may comprise elements for controlling gene expression, also called regulatory elements. Such regulatory elements may be, e.g., ribosomal binding sites, miRNA binding sites etc. The 3′-UTR may be post-transcriptionally modified, e.g. by enzymatic or post-transcriptional addition of a Poly-A tail.

Preferably, the RNA comprises a 3′-UTR, which may be derivable from a gene that relates to an RNA with enhanced half-life (i.e. that provides a stable RNA).

In some embodiments, a 3′-UTR comprises one or more of a polyadenylation signal, a binding site for proteins that affect a nucleic acid stability of location in a cell, or one or more miRNA or binding sites for miRNAs.

In preferred embodiments, the RNA of the pharmaceutical composition for multidose administration comprises at least one heterologous 3′-UTR, wherein the at least one heterologous 3′-UTR comprises a nucleic acid sequence that is derived or that is selected from a 3-UTR of a gene selected from PSMB3, ALB7, alpha-globin (referred to as “muag”), CASP1, COX6B1, GNAS, NDUFA1 and RPS9, or from a homolog, a fragment or variant of any one of these genes, preferably according to nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 253-268 of WO2021/156267, or a fragment or a variant of any of these. Particularly preferred nucleic acid sequences in that context can be derived from published PCT application WO2019/077001A1, in particular, claim 9 of WO2019/077001A1. The corresponding 3′-UTR sequences of claim 9 of WO2019/077001A1 are herewith incorporated by reference (e.g., SEQ ID NOs: 23-34 of WO2019/077001A1, or fragments or variants thereof).

In embodiments, the RNA of the pharmaceutical composition for multidose administration comprises a 3′-UTR derived from an alpha-globin gene. Said 3′-UTR derived from a alpha-globin gene (“muag”) may comprise or consist of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 267 or 268 of WO2021/156267, or a fragment or a variant thereof.

In preferred embodiments, the RNA of the pharmaceutical composition for multidose administration comprises a 3′-UTR derived from a PSMB3 gene. Said 3′-UTR derived from a PSMB3 gene may comprise or consist of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 253 or 254 of WO2021/156267, or a fragment or a variant thereof.

In other embodiments, the RNA of the pharmaceutical composition for multidose administration may comprise a 3′-UTR as described in WO2016/107877, the disclosure of WO2016/107877 relating to 3′-UTR sequences herewith incorporated by reference. Suitable 3′-UTRs are SEQ ID NOs: 1-24 and SEQ ID NOs: 49-318 of WO2016/107877, or fragments or variants of these sequences. In other embodiments, the RNA may comprise a 3′-UTR as described in WO2017/036580, the disclosure of WO2017/036580 relating to 3′-UTR sequences herewith incorporated by reference. Suitable 3′-UTRs are SEQ ID NOs: 152-204 of WO2017/036580, or fragments or variants of these sequences. In other embodiments, the RNA may comprise a 3′-UTR as described in WO2016/022914, the disclosure of WO2016/022914 relating to 3′-UTR sequences herewith incorporated by reference. Particularly preferred 3′-UTRs are nucleic acid sequences according to SEQ ID NOs: 20-36 of WO2016/022914, or fragments or variants of these sequences.

In preferred embodiments, the RNA of the pharmaceutical composition for multidose administration comprises at least one heterologous 5′-UTR.

The terms “5′-untranslated region” or “5′-UTR” or “5′-UTR element” are intended to refer to a part of an RNA molecule located 5′ (i.e. “upstream”) of a coding sequence and which is not translated into protein. A 5′-UTR may be part of an RNA located 5′ of the coding sequence. Typically, a 5′-UTR starts with the transcriptional start site and ends before the start codon of the coding sequence. A 5′-UTR may comprise elements for controlling gene expression, also called regulatory elements. Such regulatory elements may be, e.g., ribosomal binding sites, miRNA binding sites etc. The 5′-UTR may be post-transcriptionally modified, e.g. by enzymatic or post-transcriptional addition of a 5′-cap structure.

Preferably, the RNA of the pharmaceutical composition for multidose administration comprises a 5′-UTR, which may be derivable from a gene that relates to an RNA with enhanced half-life (i.e. that provides a stable RNA).

In some embodiments, a 5′-UTR comprises one or more of a binding site for proteins that affect an RNA stability or RNA location in a cell, or one or more miRNA or binding sites for miRNAs.

In preferred embodiments, the RNA of the pharmaceutical composition for multidose administration comprises at least one heterologous 5′-UTR, wherein the at least one heterologous 5′-UTR comprises a nucleic acid sequence is derived or selected from a 5′-UTR of gene selected from HSD17B4, RPL32, ASAH1, ATP5A1, MP68, NDUFA4, NOSIP, RPL31, SLC7A3, TUBB4B, and UBQLN2, or from a homolog, a fragment or variant of any one of these genes according to nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 231-252 of WO2021/156267, or a fragment or a variant of any of these.

Particularly preferred nucleic acid sequences in that context can be selected from published PCT application WO2019/077001A1, in particular, claim 9 of WO2019/077001A1. The corresponding 5′-UTR sequences of claim 9 of WO2019/077001A1 are herewith incorporated by reference (e.g., SEQ ID NOs: 1-20 of WO2019/077001A1, or fragments or variants thereof).

In preferred embodiments, the RNA of the pharmaceutical composition for multidose administration comprises a 5′-UTR derived or selected from a HSD17B4 gene, wherein said 5′-UTR derived from a HSD17B4 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 231 or 232 of WO2021/156267, or a fragment or a variant thereof.

In other embodiments, the RNA of the pharmaceutical composition for multidose administration may comprise a 5′-UTR as described in WO2013/143700, the disclosure of WO2013/143700 relating to 5′-UTR sequences herewith incorporated by reference. Particularly preferred 5′-UTRs are nucleic acid sequences derived from SEQ ID NOs: 1-1363, SEQ ID NO: 1395, SEQ ID NO: 1421 and SEQ ID NO: 1422 of WO2013/143700, or fragments or variants of these sequences. In other embodiments, the RNA may comprises a 5′-UTR as described in WO2016/107877, the disclosure of WO2016/107877 relating to 5′-UTR sequences herewith incorporated by reference. Particularly preferred 5′-UTRs are nucleic acid sequences according to SEQ ID NOs: 25-30 and SEQ ID NOs: 319-382 of WO2016/107877, or fragments or variants of these sequences. In other embodiments, the nucleic acid comprises a 5′-UTR as described in WO2017/036580, the disclosure of WO2017/036580 relating to 5′-UTR sequences herewith incorporated by reference. Particularly preferred 5′-UTRs are nucleic acid sequences according to SEQ ID NOs: 1-151 of WO2017/036580, or fragments or variants of these sequences. In other embodiments, the RNA may comprise a 5′-UTR as described in WO2016/022914, the disclosure of WO2016/022914 relating to 5′-UTR sequences herewith incorporated by reference. Particularly preferred 5′-UTRs are nucleic acid sequences according to SEQ ID NOs: 3-19 of WO2016/022914, or fragments or variants of these sequences.

In various embodiments, the RNA of the pharmaceutical composition for multidose administration may comprise a 5′-terminal sequence element according to SEQ ID NOs: 176 or 177 of WO2021/156267, or a fragment or variant thereof. Such a 5-terminal sequence element comprises e.g. a binding site for T7 RNA polymerase. Further, the first nucleotide of said 5-terminal start sequence may preferably comprise a 2′O methylation, e.g. 2′O methylated guanosine or a 2′O methylated adenosine (which is an element of a Cap1 structure).

In particularly preferred embodiments, the RNA of the pharmaceutical composition for multidose administration comprises at least one coding sequence as defined wherein said coding sequence is operably linked to a HSD17B4 5′-UTR and a PSMB3 3′-UTR (HSD17B4/PSMB3).

In particularly preferred embodiments, the RNA of the pharmaceutical composition for multidose administration comprises at least one coding sequence as defined herein, wherein said coding sequence is operably linked to an alpha-globin (“muag”) 3′-UTR.

In various embodiments, the RNA of the pharmaceutical composition for multidose administration comprises at least one poly(N) sequence, e.g. at least one poly(A) sequence, at least one poly(U) sequence, at least one poly(C) sequence, or combinations thereof.

In preferred embodiments, the RNA of the pharmaceutical composition for multidose administration comprises at least one poly(A) sequence.

The terms “poly(A) sequence”, “poly(A) tail” or “3′-poly(A) tail” as used herein will be recognized and understood by the person of ordinary skill in the art, and are e.g. intended to be a sequence of adenosine nucleotides, typically located at the 3-end of an RNA of up to about 1000 adenosine nucleotides. Preferably, said poly(A) sequence is essentially homopolymeric, e.g. a poly(A) sequence of e.g. 100 adenosine nucleotides has essentially the length of 100 nucleotides.

In other embodiments, the poly(A) sequence may be interrupted by at least one nucleotide different from an adenosine nucleotide, e.g. a poly(A) sequence of e.g. 100 adenosine nucleotides may have a length of more than 100 nucleotides (comprising 100 adenosine nucleotides and in addition said at least one nucleotide—or a stretch of nucleotides—different from an adenosine nucleotide). For example, the poly(A) sequence may comprise about 100 A nucleotides being interrupted by at least one nucleotide different from A (e.g. a linker (L), typically about 2 to 20 nucleotides in length), e.g. A30-L-A70 or A70-L-A30.

The poly(A) sequence may comprise about 10 to about 500 adenosine nucleotides, about 10 to about 200 adenosine nucleotides, about 40 to about 200 adenosine nucleotides, or about 40 to about 150 adenosine nucleotides. Suitably, the length of the poly(A) sequence may be at least about or even more than about 10, 50, 64, 75, 100, 200, 300, 400, or 500 adenosine nucleotides. In preferred embodiments, the at least one nucleic acid comprises at least one poly(A) sequence comprising about 30 to about 200 adenosine nucleotides. In particularly preferred embodiments, the poly(A) sequence comprises about 64 adenosine nucleotides (A64). In other particularly preferred embodiments, the poly(A) sequence comprises about 100 adenosine nucleotides (A100). In other embodiments, the poly(A) sequence comprises about 150 adenosine nucleotides.

The poly(A) sequence as defined herein may be located directly at the 3′ terminus of the at least one nucleic acid, preferably directly located at the 3′ terminus of an RNA. In such embodiments, the 3′-terminal nucleotide (that is the last 3′-terminal nucleotide in the polynucleotide chain) is the 3′-terminal A nucleotide of the at least one poly(A) sequence. The term “directly located at the 3′ terminus” has to be understood as being located exactly at the 3′ terminus—in other words, the 3′ terminus of the nucleic acid consists of a poly(A) sequence terminating with an A nucleotide.

In embodiments, the RNA of the pharmaceutical composition for multidose administration may comprise a poly(A) sequence obtained by enzymatic polyadenylation, wherein the majority of nucleic acid molecules comprise about 100 (+/−20) to about 500 (+/−50), preferably about 250 (+/−20) adenosine nucleotides.

In embodiments, the RNA of the pharmaceutical composition for multidose administration comprises a poly(A) sequence derived from a template DNA and additionally comprises at least one poly(A) sequence generated by enzymatic polyadenylation, e.g. as described in WO2016/091391.

In embodiments, the RNA of the pharmaceutical composition for multidose administration comprises at least one polyadenylation signal.

In embodiments, the RNA of the pharmaceutical composition for multidose administration comprises at least one poly(C) sequence.

The term “poly(C) sequence” as used herein is intended to be a sequence of cytosine nucleotides of up to about 200 cytosine nucleotides. In preferred embodiments, the poly(C) sequence comprises about 10 to about 200 cytosine nucleotides, about 10 to about 100 cytosine nucleotides, about 20 to about 70 cytosine nucleotides, about 20 to about 60 cytosine nucleotides, or about 10 to about 40 cytosine nucleotides. In a particularly preferred embodiment, the poly(C) sequence comprises about 30 cytosine nucleotides.

In embodiments, the RNA of the pharmaceutical composition for multidose administration comprises at least one histone stem-loop (hSL) or histone stem loop structure.

The term “histone stem-loop” (abbreviated as “hSL” in e.g. the sequence listing) is intended to refer to nucleic acid sequences that form a stem-loop secondary structure predominantly found in histone mRNAs.

Histone stem-loop sequences/structures may suitably be selected from histone stem-loop sequences as disclosed in WO2012/019780, the disclosure relating to histone stem-loop sequences/histone stem-loop structures incorporated herewith by reference. A histone stem-loop sequence may preferably be derived from formulae (I) or (II) of WO2012/019780. According to a further preferred embodiment, the RNA comprises at least one histone stem-loop sequence derived from at least one of the specific formulae (Ia) or (IIa) of the patent application WO2012/019780.

In preferred embodiments, the RNA of the pharmaceutical composition for multidose administration comprises at least one histone stem-loop, wherein said histone stem-loop (hSL) comprises or consists a nucleic acid sequence identical or at least 70%, 80%, 90%, 91%, 92%, 93%, 94%, 95%, 96%, 97%, 98%, or 99% identical to SEQ ID NOs: 178 or 179 of WO2021/156267, or fragments or variants thereof.

In embodiments, the RNA of the pharmaceutical composition for multidose administration comprises a 3′-terminal sequence element. Said 3′-terminal sequence element comprises a poly(A) sequence and a histone-stem-loop sequence. Accordingly, the RNA comprises at least one 3′-terminal sequence element comprising or consisting of a nucleic acid sequence being identical or at least 70%, 80%, 90%, 91%, 92%, 93%, 94%, 95%, 96%, 97%, 98%, or 99% identical to SEQ ID NOs: 182 to 230 of WO2021/156267, or a fragment or variant thereof.

In embodiments, the RNA of the pharmaceutical composition for multidose administration may be monocistronic, bicistronic, or multicistronic.

The term “monocistronic” will be recognized and understood by the person of ordinary skill in the art, and is e.g. intended to refer to an RNA that comprises only one coding sequence. The terms “bicistronic”, or “multicistronic” as used herein will be recognized and understood by the person of ordinary skill in the art, and are e.g. intended to refer to an RNA that may comprise two (bicistronic) or more (multicistronic) coding sequences.

In preferred embodiments, the RNA of the pharmaceutical composition for multidose administration is monocistronic.

In embodiments, the A/U (A/T) content in the environment of the ribosome binding site of the RNA may be increased compared to the A/U (A/T) content in the environment of the ribosome binding site of its respective wild type nucleic acid. This modification (an increased A/U (A/T) content around the ribosome binding site) increases the efficiency of ribosome binding to the RNA. An effective binding of the ribosomes to the ribosome binding site in turn has the effect of an efficient translation the RNA. Accordingly, in a particularly preferred embodiment, the RNA of the composition comprises a ribosome binding site, also referred to as “Kozak sequence” identical to or at least 80%, 85%, 90%, 95% identical to any one of the sequences SEQ ID NOs: 180 or 181 of WO2021/156267, or fragments or variants thereof.

In various embodiments the RNA comprises, preferably in 5′- to 3′-direction, the following elements:
A) 5′-cap structure, preferably as specified herein;
B) 5-terminal start element, preferably as specified herein;
C) optionally, a 5′-UTR, preferably as specified herein;
D) a ribosome binding site, preferably as specified herein;
E) at least one coding sequence, preferably as specified herein;
F) 3′-UTR, preferably as specified herein;
G) optionally, poly(A) sequence, preferably as specified herein;
H) optionally, poly(C) sequence, preferably as specified herein;
I) optionally, histone stem-loop preferably as specified herein;
J) optionally, 3-terminal sequence element, preferably as specified herein.

In particularly preferred embodiments the RNA, comprises the following elements in 5′- to 3′-direction:
A) cap1 structure as defined herein;
B) 5-terminal start element, preferably as specified herein;
C) coding sequence as specified herein;
D) 3′-UTR derived from a 3′-UTR of a muag gene as defined herein, preferably according to SEQ ID NO: 267 or 268 of WO2021/156267;
E) poly(A) sequence comprising about 64 A nucleotides.
F) poly(C) sequence comprising about 10 to about 100 cytosines;
G) histone stem-loop selected from SEQ ID NOs: 178 or 179 of WO2021/156267;

In particularly preferred embodiments the at least one nucleic acid, preferably the mRNA, comprises the following elements in 5′- to 3′-direction:
A) cap1 structure as defined herein;
B) 5-terminal start element, preferably as specified herein;
C) 5′-UTR derived from a HSD17B4 gene as defined herein, preferably according to SEQ ID NO: 231 or 232 of WO2021/156267;
D) coding sequence selected as specified herein;
E) 3-UTR derived from a 3′-UTR of a PSMB3 gene as defined herein, preferably according to SEQ ID NO: 253 or 254 of WO2021/156267;
F) optionally, a histone stem-loop selected from SEQ ID NOs: 178 or 179 of WO2021/156267;
G) poly(A) sequence comprising about 100 A nucleotides, preferably representing the 3′ terminus.

In the context of the invention, the RNA of the pharmaceutical composition for multidose administration may provide at least one coding sequence encoding a peptide or protein that is translated into a (functional) peptide or protein after administration (e.g. after administration to a subject, e.g. a human subject).

In preferred embodiments, the coding sequence of the RNA encodes at least one peptide or protein, wherein said at least one peptide or protein is selected or derived from a therapeutic peptide or protein. Accordingly, the RNA of the pharmaceutical composition for multidose administration may comprise at least one coding sequence encoding at least one peptide or protein suitable for use in treatment or prevention of a disease, disorder or condition.

In various embodiments, the length of the encoded peptide or protein, e.g. the therapeutic peptide or protein, may be at least or greater than about 20, 50, 100, 150, 200, 300, 400, 500, 600, 700, 800, 900, 1000, or 1500 amino acids.

In embodiments, the at least one (therapeutic) peptide or protein is selected or is derived from an antibody, an intrabody, a receptor, a receptor agonist, a receptor antagonist, a binding protein, a CRISPR-associated endonuclease, a chaperone, a transporter protein, an ion channel, a membrane protein, a secreted protein, a transcription factor, an enzyme, a peptide or protein hormone, a growth factor, a structural protein, a cytoplasmic protein, a cytoskeletal protein, a viral antigen, a bacterial antigen, a protozoan antigen, an allergen, a tumor antigen, or fragments, variants, or combinations of any of these.

In embodiments, the peptide or protein is selected from an antigen or epitope of a pandemic pathogen, preferably a pandemic virus (e.g. a pandemic Coronavirus).

In embodiments, the peptide or protein is selected from an antigen or epitope of a pathogen selected or derived from List 1 provided below.

### List 1: Suitable Pathogens of the Invention

Acinetobacter baumannii, Anaplasma genus, Anaplasma phagocytophilum, Ancylostoma braziliense, Ancylostoma duodenale, Arcanobacterium haemolyticum, Ascaris lumbricoides, Aspergillus genus, Astroviridae, Babesia genus, Bacillus anthracis, Bacillus cereus, Bartonella henselae, BK virus, Blastocystis hominis, Blastomyces dermatitidis, Bordetella pertussis, Borrelia burgdorferi, Borrelia genus, Borrelia spp, Brucella genus, Brugia malayi, Bunyaviridae family, Burkholderia cepacia and other Burkholderia species, Burkholderia mallei, Burkholderia pseudomallei, Caliciviridae family, Campylobacter genus, Candida albicans, Candida spp, Chlamydia trachomatis, Chlamydophila pneumoniae, Chlamydophila psittaci, CJD prion, Clonorchis sinensis, Clostridium botulinum, Clostridium difficile, Clostridium perfringens, Clostridium perfringens, Clostridium spp, Clostridium tetani, Coccidioides spp, coronaviruses, Corynebacterium diphtheriae, Coxiella burnetii, Crimean-Congo hemorrhagic fever virus, Cryptococcus neoformans, Cryptosporidium genus, Cytomegalovirus (CMV), Dengue viruses (DEN-1, DEN-2, DEN-3 and DEN-4), Dientamoeba fragilis, Ebolavirus (EBOV), Echinococcus genus, Ehrlichia chaffeensis, Ehrlichia ewingii, Ehrlichia genus, Entamoeba histolytica, Enterococcus genus, Enterovirus genus, Enteroviruses, mainly Coxsackie A virus and Enterovirus 71 (EV71), Epidermophyton spp, Epstein-Barr Virus (EBV), Escherichia coli O157:H7, O111 and O104:H4, Fasciola hepatica and Fasciola gigantica, FFI prion, Filarioidea superfamily, Flaviviruses, Francisella tularensis, Fusobacterium genus, Geotrichum candidum, Giardia intestinalis, Gnathostoma spp, GSS prion, Guanarito virus, Haemophilus ducreyi, Haemophilus influenzae, Helicobacter pylori, Henipavirus (Hendra virus Nipah virus), Hepatitis A Virus, Hepatitis B Virus (HBV), Hepatitis C Virus (HCV), Hepatitis D Virus, Hepatitis E Virus, Herpes simplex virus 1 and 2 (HSV-1 and HSV-2), Histoplasma capsulatum, HIV (Human immunodeficiency virus), Hortaea werneckii, Human bocavirus (HBoV), Human herpesvirus 6 (HHV-6) and Human herpesvirus 7 (HHV-7), Human metapneumovirus (hMPV), Human papillomavirus (HPV), Human parainfluenza viruses (HPIV), Japanese encephalitis virus, JC virus, Junin virus, Kingella kingae, Klebsiella granulomatis, Kuru prion, Lassa virus, Legionella pneumophila, Leishmania genus, Leptospira genus, Listeria monocytogenes, Lymphocytic choriomeningitis virus (LCMV), Machupo virus, Malassezia spp, Marburg virus, Measles virus, Metagonimus yokagawai, Microsporidia phylum, Molluscum contagiosum virus (MCV), Mumps virus, Mycobacterium leprae and Mycobacterium lepromatosis, Mycobacterium tuberculosis, Mycobacterium ulcerans, Mycoplasma pneumoniae, Naegleria fowleri, Necator americanus, Neisseria gonorrhoeae, Neisseria meningitidis, Nocardia asteroides, Nocardia spp, Onchocerca volvulus, Orientia tsutsugamushi, Orthomyxoviridae family (Influenza), Paracoccidioides brasiliensis, Paragonimus spp, Paragonimus westermani, Parvovirus B19, Pasteurella genus, Plasmodium genus, Pneumocystis jirovecii, Poliovirus, Rabies virus, Respiratory syncytial virus (RSV), Rhinovirus, rhinoviruses, Rickettsia akari, Rickettsia genus, Rickettsia prowazekii, Rickettsia rickettsii, Rickettsia typhi, Rift Valley fever virus, Rotavirus, Rubella virus, Sabia virus, Salmonella genus, Sarcoptes scabiei, SARS coronavirus, SARS-CoV-2 coronavirus, Schistosoma genus, Shigella genus, Sin Nombre virus, Hantavirus, Sporothrix schenckii, Staphylococcus genus, Staphylococcus genus, Streptococcus agalactiae, Streptococcus pneumoniae, Streptococcus pyogenes, Strongyloides stercoralis, Taenia genus, Taenia solium, Tick-borne encephalitis virus (TBEV), Toxocara canis or Toxocara cati, Toxoplasma gondii, Treponema pallidum, Trichinella spiralis, Trichomonas vaginalis, Trichophyton spp, Trichuris trichiura, Trypanosoma brucei, Trypanosoma cruzi, Ureaplasma urealyticum, Varicella zoster virus (VZV), Varicella zoster virus (VZV), Variola major or Variola minor, vCJD prion, Venezuelan equine encephalitis virus, Vibrio cholerae, West Nile virus, Western equine encephalitis virus, Wuchereria bancrofti, Yellow fever virus, Yersinia enterocolitica, Yersinia pestis, and Yersinia pseudotuberculosis.

In embodiments, the peptide or protein is selected from an antigen or epitope of a pathogen selected or derived from a (pandemic) Coronavirus, e.g. SARS-CoV-2, or a fragment or variant of any of these.

According to preferred embodiments, the RNA of the pharmaceutical composition for multidose administration comprises a coding sequence encoding at least one antigen or epitope selected or derived from a (pandemic) Coronavirus, preferably SARS-CoV-2.

In preferred embodiments, the at least one antigenic or epitope selected or derived from a (pandemic) Coronavirus comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 1-111, 274-11663, 13176-13510, 13521-14123, 22732-22758, 22917, 22923, 22929-22964, 26938, 26939 of WO2021156267, or an immunogenic fragment or immunogenic variant of any of these amino acid sequences.

In preferred embodiments, the RNA comprises at least one coding sequence comprising or consisting of at least one nucleic acid sequence which is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 116-132, 134-138, 140-143, 145-175, 11664-11813, 11815, 11817-12050, 12052, 12054-13147, 13514, 13515, 13519, 13520, 14124-14177, 22759, 22764-22786, 22791-22813, 22818-22839, 22969-23184, 23189-23404, 23409-23624, 23629-23844, 23849-24064, 24069-24284, 24289-24504, 24509-24724, 24729-24944, 24949-25164, 25169-25384, 25389-25604, 25609-25824, 25829-26044, 26049-26264, 26269-26484, 26489-26704, 26709-26937 of WO2021156267, or a fragment or a fragment or variant of any of these nucleic acid sequences

In preferred embodiments, the mRNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 148-175, 12204-13147, 14142-14177, 22786-22839, 23189-23404, 23409-23624, 23629-23844, 23849-24064, 24069-24284, 24289-24504, 24509-24724, 24729-24944, 24949-25164, 25169-25384, 25389-25604, 25609-25824, 25829-26044, 26049-26264, 26269-26484, 26489-26704, 26709-26937 of WO2021156267, or a fragment or a fragment or variant of any of these RNA sequences

In a preferred embodiment, the RNA encoding the antigen or epitope selected or derived from a SARS-CoV-2 virus comprises or consists of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NO: 3 or a fragment or variant of that sequence. Preferably, the RNA sequence does not comprise chemically modified nucleotides. Preferably, the RNA comprises a 5′ cap1 structure.

In embodiments, the peptide or protein is selected from an antigen or epitope of a pathogen selected or derived from a Rabies virus, or a fragment or variant of any of these.

According to preferred embodiments, the RNA of the pharmaceutical composition for multidose administration comprises a coding sequence encoding at least one antigen or epitope selected or derived from a Rabies virus.

In a preferred embodiment, the RNA encoding the antigen or epitope selected or derived from a Rabies virus comprises or consists of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NO: 6 or a fragment or variant of any of these sequences. Preferably, the RNA sequence does not comprise chemically modified nucleotides. Preferably, the RNA comprises a 5′ cap1 structure.

In preferred embodiments, the pharmaceutical composition for multidose administration is a liquid pharmaceutical composition.

In embodiments, the concentration of lipid (or lipid-based carriers) in the pharmaceutical composition for multidose administration is in a range from about 250 μg/ml to about 250 mg/ml.

In embodiments, the concentration of lipid (or lipid-based carriers) in the pharmaceutical composition for multidose administration is for example about 2.5 mg/ml, about 5 mg/ml, about 7.5 mg/ml, about 10 mg/ml, about 12.5 mg/ml, about 15 mg/ml, about 17.5 mg/ml, about 20 mg/ml, about 22.5 mg/ml, or about 25 mg/ml.

In that context, “the concentration of lipid (or lipid-based carriers)” relates to the total concentration of lipid (or lipid-based carriers) in the composition.

In embodiments, the weight to weight (wt/wt) ratio of lipid to the RNA (in the lipid-based carriers) is from about 10:1 to about 60:1. In preferred embodiments, the weight to weight (wt/wt) ratio of lipid to the RNA (in the lipid-based carriers) is from about 20:1 to about 30:1. In embodiments, the weight to weight (wt/wt) ratio of lipid to the RNA (in the lipid-based carriers) is for example about 20:1, about 21:1, about 22:1, about 23:1, about 24:1, about 25:1, about 26:1, about 27:1, about 28:1, about 29:1, or about 30:1. In particularly preferred embodiments, the wt/wt ratio of lipid to the RNA (in the lipid-based carriers) is about 25:1.

In embodiments, the RNA to total lipid ratio in the lipid based carriers is less than about 0.1 w/w, preferably less than about 0.06 w/w. In preferred embodiments, the RNA to total lipid ratio in the lipid based carriers is between about 0.03 w/w and 0.05 w/w. In particularly preferred embodiments, the RNA to total lipid ratio in the lipid based carriers is between about 0.04 w/w.

The amount of lipid comprised in the lipid-based carriers may be selected taking the amount of the RNA cargo into account.

In one embodiment, these amounts are selected such as to result in an N/P ratio of the lipid-based carriers encapsulating the RNA in the range of about 0.1 to about 20. The N/P ratio is defined as the mole ratio of the nitrogen atoms (“N”) of the basic nitrogen-containing groups of the lipid to the phosphate groups (“P”) of the RNA which is used as cargo. The N/P ratio may be calculated on the basis that, for example, 1 ug RNA typically contains about 3 nmol phosphate residues, provided that the RNA exhibits a statistical distribution of bases. The “N”-value of the lipid or lipidoid may be calculated on the basis of its molecular weight and the relative content of permanently cationic and—if present—cationisable groups.

In embodiments, the N/P ratio of the lipid-based carriers to the RNA is in a range from about 1 to about 10, preferably in a range from about 1 to about 7, more preferably in a range from about 5 to about 7, e.g. about 5.5, about 5.6, about 5.7, about 5.8, about 5.9, about 6, about 6.1, about 6.2, bout 6.3, about 6.4, about 6.5. In preferred embodiments, the N/P ratio of the lipid-based carriers to the RNA is about 6.

In various embodiments, lipid-based carriers encapsulating the RNA are monodisperse, meaning that the lipid-based carriers comprised in the composition have a uniform size. Typically, the distribution of size populations within a composition is expressed by the polydispersity index (PDI) value.

The term “polydispersity index” (PDI) is used herein as a measure of the size distribution of an ensemble of particles, e.g., lipid-based carriers. The polydispersity index is calculated based on dynamic light scattering measurements by the so-called cumulant analysis. Typically, the PDI is determined by dynamic light scattering at an angle of 90° or 173°, typically measured at a temperature of 25° C. PDI is basically a representation of the distribution of size populations within a given sample. The numerical value of PDI ranges from 0.0 (for a perfectly uniform sample with respect to the particle size) to 1.0 (for a highly polydisperse sample with multiple particle size populations).

In embodiments, the lipid-based carriers encapsulating the RNA have as a polydispersity index (PDI) value ranging from about 0.50 to about 0.00. In embodiments, the lipid-based carriers encapsulating the RNA have a polydispersity index (PDI) value of less than about 0.3, preferably of less than about 0.2, more preferably of less than about 0.15, most preferably of less than about 0.1.

In various embodiments, the pharmaceutical composition for multidose administration comprises lipid-based carriers encapsulating the RNA that have a defined size (particle size, homogeneous size distribution).

The size of the lipid-based carriers encapsulating the RNA is typically described herein as Z-average size. The terms “average diameter”, “mean diameter”, “diameter” or “size” for particles (e.g. lipid-based carrier) are used synonymously with the value of the Z-average. The term “Z-average size” refers to the mean diameter of particles as measured by dynamic light scattering (DLS) with data analysis using the so-called cumulant algorithm, which provides as results the so-called Z-average with the dimension of a length, and the polydispersity index (PI), which is dimensionless (Koppel, D., J. Chem. Phys. 57, 1972, pp 4814-4820, ISO 13321).

The term “dynamic light scattering” or “DLS” refers to a method for analyzing particles in a liquid, wherein the liquid is typically illuminated with a monochromatic light source and wherein the light scattered by particles in the liquid is detected.

Due to Brownian motion, smaller particles typically result in time-dependent scattering intensity fluctuations that are distinct from those observed for larger particles. DLS can thus be used to measure particle sizes in a liquid. Suitable DLS protocols are known in the art. DLS instruments are commercially available (such as the Zetasizer Nano Series, Malvern Instruments, Worcestershire, UK). DLS instruments employ either a detector at 90° (e.g., DynaPro® NanoStar® from Wyatt Technology or Zetasizer Nano S90® from Malvern Instruments) or a backscatter detection system at 173° (e.g., Zetasizer Nano S® from Malvern Instruments) and at 158° (DynaPro Plate Reader® from Malvern Instruments) close to the incident light of 180°. Typically, DLS measurements are performed at a temperature of about 25° C. DLS is also used in the context of the present invention to determine the polydispersity index (PDI) and/or the main peak diameter of the lipid-based carriers incorporating RNA.

An alternative to DLS is nanoparticle tracking analysis (NTA) or micro-flow imaging (MFI).

Nanoparticle tracking analysis (NTA) refers to a method for analyzing particles in a liquid that relates the rate of Brownian motion to particle size. Suitable NTA protocols are known in the art and instruments for NTA are commercially available (such as the NanoSight instruments, e.g. NanoSight LM20, NanoSight, Amesbury, UK).

Micro-flow imaging (MFI) is a flow microscopy technology, where bright field images are captured in successive frames as a continuous sample stream passes through a flow cell centered in the field-of-view of a custom magnification system having a well-characterized and extended depth-of-field. MFI may be used to analyse the amount or concentration of sub-visible particles comprised in the composition (larger than about 500 nm, e.g. measured by a Coulter counter device).

In various embodiments, the lipid-based carriers encapsulating the RNA have a Z-average size ranging from about 50 nm to about 200 nm, from about 50 nm to about 190 nm, from about 50 nm to about 180 nm, from about 50 nm to about 170 nm, from about 50 nm to about 160 nm, 50 nm to about 150 nm, 50 nm to about 140 nm, 50 nm to about 130 nm, 50 nm to about 120 nm, 50 nm to about 110 nm, 50 nm to about 100 nm, 50 nm to about 90 nm, 50 nm to about 80 nm, 50 nm to about 70 nm, 50 nm to about 60 nm, 60 nm to about 200 nm, from about 60 nm to about 190 nm, from about 60 nm to about 180 nm, from about 60 nm to about 170 nm, from about 60 nm to about 160 nm, 60 nm to about 150 nm, 60 nm to about 140 nm, 60 nm to about 130 nm, 60 nm to about 120 nm, 60 nm to about 110 nm, 60 nm to about 100 nm, 60 nm to about 90 nm, 60 nm to about 80 nm, or 60 nm to about 70 nm, for example about 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 160 nm, 170 nm, 180 nm, 190 nm, or 200 nm. Suitably, the Z-average size may be determined by DLS as commonly known in the art.

In preferred embodiments, the lipid-based carriers encapsulating the RNA have a Z-average size ranging from about 50 nm to about 150 nm, preferably in a range from about 50 nm to about 120 nm, more preferably in a range from about 60 nm to about 115 nm. Suitably, the Z-average size may be determined by DLS as commonly known in the art.

In embodiments, the lipid-based carriers encapsulating the RNA have a Z-average size of less than about 150 nm, preferably less than about 120 nm, more preferably less than about 100 nm, most preferably less than about 80 nm. Suitably, the Z-average size may be determined by DLS as commonly known in the art.

In embodiments, the lipid-based carriers encapsulating the RNA comprise more than about 70%, 75%, 80%, 85%, 90%, 91%, 92%, 93%, 94%, 95%, 96%, 97%, 98%, 99% lipid-based carriers that have a particle size ranging from about 50 nm to about 150 nm, preferably ranging from about 60 nm to about 115 nm, more preferably ranging from about 60 nm to about 80 nm. The particle size may be determined by DLS as commonly known in the art (e.g. MADLS). Alternatively, nanoparticle tracking analysis (NTA) or MFI as commonly known in the art may be used, or electron microscopy may be used (determining the particle size of lipid-based carriers comprised in a certain sample volume of the composition).

In embodiments, the lipid-based carriers encapsulating the RNA comprise less than about 10%, 9%, 8%, 7%, 6%, 5%, 4%, 3%, 2%, 1% lipid-based carriers that have a particle size exceeding about 500 nm. The particle size may be determined by DLS as commonly known in the art (e.g. MADLS). Alternatively, nanoparticle tracking analysis (NTA) or MFI as commonly known in the art may be used, or electron microscopy may be used (determining the particle size of lipid-based carriers comprised in a certain sample volume of the composition).

In embodiments, the pharmaceutical composition for multidose administration comprising the lipid-based carriers encapsulating the RNA comprises less than about 200,000 subvisible particles ≥2 μm (#per ml). In embodiments, the pharmaceutical composition for multidose administration comprising the lipid-based carriers encapsulating the RNA comprises less than about 100,000 subvisible particles ≥2 μm (#per ml). Preferably, the number of subvisible particles is determined by MFI.

In embodiments, the Zeta potential of the lipid-based carriers encapsulating the RNA is in a range from +20 mV to −20 mV, preferably from +10 mV to −10 mV, more preferably at around 0 mV. Methods for determining the Zeta potential are known in the art. For example, the Zeta potential of the lipid-based carriers encapsulating the RNA can be determined by using a Zetasizer (Malvern Instruments, Worcestershire, UK).

In embodiments, the lipid-based carriers encapsulating the RNA are a liposomes, lipid nanoparticles, lipoplexes, and/or nanoliposomes.

In preferred embodiments, the lipid-based carriers encapsulating the RNA are a lipid nanoparticle (LNP).

Accordingly, the RNA may be completely or partially encapsulated in a lipid nanoparticle, wherein the RNA may be located in the interior space of the lipid nanoparticle, within the lipid layer/membrane of the lipid nanoparticle, or associated with the exterior surface of the lipid nanoparticle. Lipid nanoparticles (LNPs) are typically characterized as microscopic lipid particles having a solid (lipid) core or partially solid (lipid) core. Typically, an LNP does not comprise an interior aqua space sequestered from an outer medium by a bilayer. However, an LNP may comprise multiple internal (aqueous) droplets in the core of a lipid nanoparticle that may entrap the RNA. In an LNP, the RNA may suitably be encapsulated or incorporated in the lipid portion of the LNP enveloped by some or the entire lipid portion of the LNP. An LNP may comprise any lipid combination capable of forming a particle to which the RNA may be attached, or in which the RNA may be encapsulated.

In preferred embodiments, the lipid-based carriers (e.g. LNPs) encapsulating the RNA comprise at least two lipid components, at least three lipid components, preferably at least four lipid components, wherein the lipid components may be selected from at least one aggregation-reducing lipid, at least one cationic lipid, at least one neutral lipid, and/or at least one steroid or steroid analog.

In preferred embodiments, the lipid-based carriers (e.g. LNPs) encapsulating the RNA comprise at least one aggregation-reducing lipid, at least one cationic lipid, at least one neutral lipid, and/or at least one steroid or steroid analog.

In embodiments, the lipid-based carriers encapsulating the RNA comprise at least one aggregation reducing lipid.

In embodiments, the lipid-based carriers encapsulating the RNA comprise the aggregation reducing lipid (e.g. polymer-conjugated lipid) in a molar ratio of about 0.5% to about 15%, preferably in a molar ratio of about 1.0% to about 2.5%, for example in a molar ratio of about 1.4%, about 1.5%, about 1.6%, about 1.7%, about 1.8%, about 1.9%. In preferred embodiments, the lipid-based carriers encapsulating the RNA comprise the aggregation reducing lipid in a molar ratio of about 1.7% (based on 100% total moles of lipids in the lipid-based carriers).

In embodiments, the lipid-based carriers encapsulating the RNA comprise the aggregation reducing lipid in a weight ratio of about 2% to about 10%, preferably in a weight ratio of about 4% to about 10%, for example in a weight ratio of about 5%, about 6%, about 7%, about 8%, about 9%. In embodiments, the lipid-based carriers encapsulating the RNA comprise the aggregation reducing lipid in a weight ratio of about 6.97% (based on 100% total weight of lipids in the lipid-based carriers). The term “aggregation reducing lipid” refers to a molecule comprising both a lipid portion and a moiety suitable of reducing or preventing aggregation of the lipid-based carriers encapsulating the RNA in a composition. Under storage conditions, the lipid-based carriers may undergo charge-induced aggregation, a condition which can be undesirable for the stability of the composition. Therefore, it can be desirable to include a lipid compound which can reduce aggregation, for example by sterically stabilizing the lipid-based carriers. Such a steric stabilization may occur when a compound having a sterically bulky but uncharged moiety that shields or screens the charged portions of a lipid-based carriers from close approach to other lipid-based carriers in the composition. In the context of the invention, stabilization of the lipid-based carriers is achieved by including lipids which may comprise a lipid bearing a sterically bulky group which, after formation of the lipid-based carrier, is preferably located on the exterior of the lipid-based carrier. Suitable aggregation reducing groups include hydrophilic groups, e.g. polymers, such as poly(oxyalkylenes), e.g., a poly(ethylene glycol) or poly(propylene glycol). Lipids comprising a polymer as aggregation reducing group are herein referred to as “polymer conjugated lipid”.

In embodiments, the aggregation reducing lipid of the lipid-based carriers encapsulating the RNA is a polymer conjugated lipid.

The term “polymer conjugated lipid” refers to a molecule comprising both a lipid portion and a polymer portion, wherein the polymer is suitable of reducing or preventing aggregation of lipid-based carriers encapsulating the RNA in the composition. A polymer has to be understood as a substance or material consisting of very large molecules, or macromolecules, composed of many repeating subunits. A suitable polymer in the context of the invention may be a hydrophilic polymer. An example of a polymer conjugated lipid is a PEGylated or PEG-conjugated lipid.

In embodiments, the aggregation reducing lipid is selected from a polymer conjugated lipid. In preferred embodiments, the polymer conjugated lipid is a PEG-conjugated lipid (or PEGylated lipid or PEG lipid).

In certain embodiments, the lipid-based carriers encapsulating the RNA comprise a polyethylene glycol-lipid (PEG-conjugated) which may a stabilize the pharmaceutical composition for multidose administration. Suitable polyethylene glycol-lipids include PEG-conjugated phosphatidylethanolamine, PEG-conjugated phosphatidic acid, PEG-conjugated ceramides (e.g. PEG-CerC14 or PEG-CerC20), PEG-conjugated dialkylamines, PEG-conjugated diacylglycerols, PEG-conjugated dialkylglycerols. Representative polyethylene glycol-lipids include PEG-c-DOMG, PEG-c-DMA, and PEG-s-DMG. In one embodiment, the polyethylene glycol-lipid is N-[(methoxy poly(ethylene glycol)2000)carbamyl]-1,2-dimyristyloxlpropyl-3-amine (PEG-c-DMA). In a preferred embodiment, the polyethylene glycol-lipid is PEG-2000-DMG. In one embodiment, the polyethylene glycol-lipid is PEG-c-DOMG). In other embodiments, the lipid-based carriers comprise a PEG-conjugated diacylglycerol (PEG-DAG) such as 1-(monomethoxy-polyethyleneglycol)-2,3-dimyristoylglycerol (PEG-DMG), a PEG-conjugated phosphatidylethanoloamine (PEG-PE), a PEG-conjugated succinate diacylglycerol (PEG-S-DAG) such as 4-O-(2′,3′-di(tetradecanoyloxy)propyl-1-O-(ω-methoxy(polyethoxy)ethyl)butanedioate (PEG-S-DMG), a PEG-conjugated ceramide (PEG-cer), or a PEG-conjugated dialkoxypropylcarbamate such as w-methoxy(polyethoxy)ethyl-N-(2,3di(tetradecanoxy)propyl)carbamate or 2,3-di(tetradecanoxy)propyl-N-(ω-methoxy(polyethoxy)ethyl)carbamate.

In embodiments, the polymer conjugated lipid, e.g. the PEG-conjugated lipid is preferably derived from formula (IV) of published PCT patent application WO2018/078053A1. Accordingly, the PEG-conjugated lipids derived from formula (IV) of published PCT patent application WO2018/078053A1, and the respective disclosure relating thereto, are herewith incorporated by reference.

In preferred embodiments, the lipid-based carriers (e.g. the LNPs) encapsulating the RNA comprise a polymer conjugated lipid, preferably a PEG-conjugated, wherein the a PEG-conjugated lipid is preferably derived from formula (IVa) of published PCT patent application WO2018/078053A1. Accordingly, a PEG-conjugated lipids derived from formula (IVa) of published PCT patent application WO2018/078053A1, and the respective disclosure relating thereto, are herewith incorporated by reference.

In preferred embodiments, the lipid-based carriers (e.g. the LNPs) encapsulating the RNA comprise a PEG-conjugated lipid, wherein said PEG-conjugated lipid is a lipid according to formula (IVa) or derived from formula (IVa):

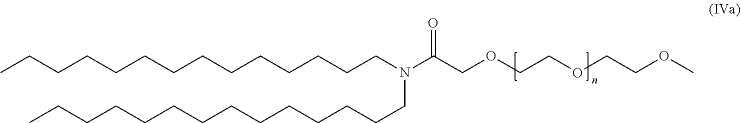

wherein n has a mean value ranging from 30 to 60, such as about 30±2, 32±2, 34±2, 36±2, 38±2, 40±2, 42±2, 44±2, 46±2, 48±2, 50±2, 52±2, 54±2, 56±2 58±2, or 60±2. In a preferred embodiment n is about 49. In another preferred embodiment n is about 45.

Further examples of PEG-conjugated lipids suitable in that context are provided in US2015/0376115A1 and WO2015/199952, each of which is incorporated by reference in its entirety.

Furthermore, in a specific embodiment, the lipid based carrier encapsulating the RNA comprise a polymer conjugated lipid, preferably a PEG-conjugated lipid, wherein said PEG-conjugated lipid is 1,2-dimyristoyl-rac-glycero-3-methoxypolyethylene glycol 2000 (DMG-PEG 2000) according to or derived from the following structure:

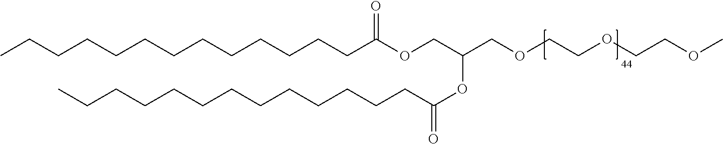

As used in the art, “DMG-PEG 2000” is considered a mixture of 1,2-DMG PEG2000 and 1,3-DMG PEG2000 in ˜97:3 ratio.

In preferred embodiments, the lipid-based carriers encapsulating the RNA comprise at least one cationic lipid.

In embodiments, the lipid-based carriers encapsulating the RNA comprise the cationic lipid in a molar ratio of about 20% to about 60%, preferably in a molar ratio of about 38% to about 57%, for example in a molar ratio of about 42%, 43%, 44%, 45%, 46%, 47%, 48%, 49%, 50%, 51%, or about 52% (based on 100% total moles of lipids in the lipid-based carriers). In preferred embodiments, the lipid-based carriers encapsulating the RNA comprise a cationic lipid in a molar ratio of about 47.4% (based on 100% total moles of lipids in the lipid-based carriers).

In embodiments, the lipid-based carriers encapsulating the RNA comprise the cationic lipid in a weight ratio of about 24% to about 72%, preferably in a weight ratio of about 45% to about 68%, for example in a weight ratio of about 50%, 51%, 52%, 53%, 54%, 55%, 56%, 57%, 58%, 59%, 60%, 61%, or about 62% (based on 100% total weight of lipids in the lipid-based carriers). In preferred embodiments, the lipid-based carriers encapsulating the RNA comprise the cationic lipid in a weight ratio of about 56.28% (based on 100% total weight of lipids in the lipid-based carriers).

The cationic lipid of the lipid-based carriers encapsulating the RNA may be cationisable, i.e. it becomes protonated as the pH is lowered below the pK of the ionizable group of the lipid, but is progressively more neutral at higher pH values. At pH values below the pK, the lipid is then able to associate with negatively charged nucleic acids. In certain embodiments, the cationic lipid comprises a zwitterionic lipid that assumes a positive charge on pH decrease.

Suitable cationic lipids or cationisable lipids include, but are not limited to, DSDMA, N,N-dioleyl-N,N-dimethylammonium chloride (DODAC), N,N-distearyl-N,N-dimethylammonium bromide (DDAB), 1,2-dioleoyltrimethyl ammonium propane chloride (DOTAP) (also known as N-(2,3-dioleoyloxy)propyl)-N,N,N-trimethylammonium chloride and 1,2-Dioleyloxy-3-trimethylaminopropane chloride salt), N-(1-(2,3-dioleyloxy)propyl)-N,N,N-trimethylammonium chloride (DOTMA), N,N-dimethyl-2,3-dioleyloxy)propylamine (DODMA), ckk-E12, ckk, 1,2-DiLinoleyloxy-N,N-dimethylaminopropane (DLinDMA), 1,2-Dilinolenyloxy-N,N-dimethylaminopropane (DLenDMA), 1,2-di-y-linolenyloxy-N,N-dimethylaminopropane (γ-DLenDMA), 98N12-5,1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), ICE (Imidazol-based), HGT5000, HGT5001, DMDMA, CLinDMA, CpLinDMA, DMOBA, DOcarbDAP, DLincarbDAP, DLinCDAP, KLin-K-DMA, DLin-K-XTC2-DMA, XTC (2,2-Dilinoleyl-4-dimethylaminoethyl-[1,3]-dioxolane) HGT4003, 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DM A), 2,2-Dilinoleyl-4-dimethylaminomethyl-[1,3]-dioxolane (DLin-K-DMA) or analogs thereof, (3aR,5s,6aS)—N,N-dimethyl-2,2-di((9Z,12Z)-octadeca-9,12-dienyl)tetrahydro-3aH-cyclopenta[d][1,3]dioxol-5-amine, (6Z,9Z,28Z,31Z)-heptatriaconta-6,9,28,31-tetraen-19-yl-4-(dimethylamino)butanoate (MC3), ALNY-100 ((3aR,5s,6aS)—N,N-dimethyl-2,2-di((9Z,12Z)-octadeca-9,12-dienyl)tetrahydro-3aH-cyclopenta[d] [1,3]dioxol-5-amine)), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (C12-200), 2,2-dilinoleyl-4-(2-dimethylaminoethyl)-[1,3]-dioxolane (DLin-K-C2-DMA), 2,2-dilinoleyl-4-dimethylaminomethyl-[1,3]-dioxolane (DLin-K-DMA), NC98-5 (4,7,13-tris(3-oxo-3-(undecylamino)propyl)-N,N 16-diundecyl-4,7,10,13-tetraazahexadecane-1,16-diamide), (6Z,9Z,28Z,31Z)-heptatriaconta-6,9,28,31-tetraen-19-yl 4-(dimethylamino) butanoate (DLin-M-C3-DMA), 3-((6Z,9Z,28Z,31Z)-heptatriaconta-6,9,28,31-tetraen-19-yloxy)-N,N-dimethylpropan-1-amine (MC3 Ether), 4-((6Z,9Z,28Z,31Z)-heptatriaconta-6,9,28,31-tetraen-19-yloxy)-N,N-dimethylbutan-1-amine (MC4 Ether), LIPOFECTIN® (commercially available cationic liposomes comprising DOTMA and 1,2-dioleoyl-sn-3phosphoethanolamine (DOPE), from GIBCO/BRL, Grand Island, N.Y.); LIPOFECTAMINE® (commercially available cationic liposomes comprising N-(1-(2,3dioleyloxy)propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.) or any combination of any of the foregoing. Further suitable cationic or cationizable lipids include those described in international patent publications WO2010/053572 (and particularly, Cl 2-200 described at paragraph [00225]) and WO2012/170930, both of which are incorporated herein by reference, HGT4003, HGT5000, HGTS001, HGT5001, HGT5002 (see US20150140070A1).

In embodiments, the cationic lipid of the lipid-based carriers encapsulating the RNA is selected from at least one amino lipid.

Suitable amino lipids include, but are not limited to, 1,2-dilinoleyoxy-3-(dimethylamino)acetoxypropane (DLin-DAC), 1,2-dilinoleyoxy-3morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (DLin-MPZ), 3-(N,Ndilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA), 2,2-dilinoleyl-4-(2-dimethylaminoethyl)-[1,3]-dioxolane (DLin-KC2-DMA); dilinoleyl-methyl-4-dimethylaminobutyrate (DLin-MC3-DMA); MC3 (US20100324120).

In embodiments, the cationic or cationizable lipid of the lipid-based carriers encapsulating the RNA is selected from at least one aminoalcohol lipidoid.

Aminoalcohol lipidoids which may be used in the present invention may be prepared by the methods described in U.S. Pat. No. 8,450,298, herein incorporated by reference in its entirety. Suitable (ionizable) lipids can also be the compounds as disclosed in Tables 1, 2 and 3 and as defined in claims 1-24 of WO2017/075531A1, hereby incorporated by reference.

In other embodiments, suitable cationic or cationizable lipids may be selected from compounds as disclosed in WO2015/074085A1 (i.e. ATX-001 to ATX-032 or the compounds as specified in claims 1-26), U.S. Appl. Nos. 61/905,724 and Ser. No. 15/614,499 or U.S. Pat. Nos. 9,593,077 and 9,567,296 hereby incorporated by reference in their entirety. In other embodiments, suitable cationic or cationizable lipids may be selected from compounds as disclosed in WO2017/117530A1 (i.e. lipids 13, 14, 15, 16, 17, 18, 19, 20, or the compounds as specified in the claims), hereby incorporated by reference in its entirety.

In preferred embodiments, cationic or cationizable lipids may be selected from the lipids disclosed in WO2018/078053A1 (i.e. lipids derived from formula I, II, and III of WO2018/078053A1, or lipids as specified in Claims 1 to 12 of WO2018/078053A1), the disclosure of WO2018/078053A1 hereby incorporated by reference in its entirety. In that context, lipids disclosed in Table 7 of WO2018/078053A1 (e.g. lipids derived from formula I-1 to 1-41) and lipids disclosed in Table 8 of WO2018/078053A1 (e.g. lipids derived from formula II-1 to II-36) may be suitably used. Accordingly, formula I-1 to formula I-41 and formula II-1 to formula II-36 of WO2018/078053A1, and the specific disclosure relating thereto, are herewith incorporated by reference.

In preferred embodiments, suitable cationic or cationizable lipids may be derived from formula III of published PCT patent application WO2018/078053A1. Accordingly, formula III of WO2018/078053A1, and the specific disclosure relating thereto, are herewith incorporated by reference.

In particularly preferred embodiments, the lipid-based carriers (e.g. LNPs) encapsulating the RNA comprise a cationic lipid selected from or derived from structures III-1 to 111-36 of Table 9 of published PCT patent application WO2018/078053A1. Accordingly, formula III-1 to III-36 of WO2018/078053A1, and the specific disclosure relating thereto, are herewith incorporated by reference.

In particularly preferred embodiments, the lipid-based carriers (e.g. LNPs) encapsulating the RNA comprise a cationic lipid according to formula (III-3) or derived from formula (III-3):

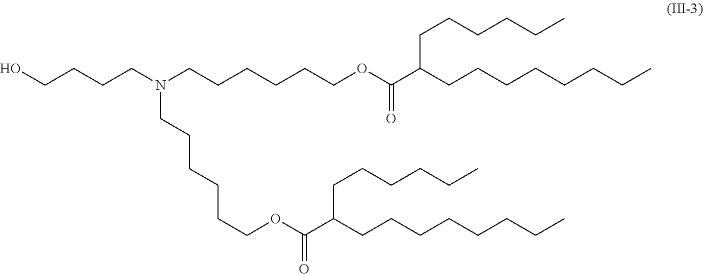 [SMILES: CCCCCCCCC(CCCCCC)C(=O)OCCCCCCN(CCCCO)CCCCCCOC(=O)C(CCCCCC)CCCCCCCC]

In certain embodiments, the cationic lipid as defined herein, more preferably cationic lipid compound III-3, is present in the lipid-based carriers encapsulating the RNA in an amount from about 30 to about 95 mole percent, relative to the total lipid content of the lipid-based carrier. If more than one cationic lipid is incorporated within the lipid-based carrier, such percentages apply to the combined cationic lipids.

In an embodiment, the lipid-based carriers encapsulating the RNA comprise a cationic lipid resembled by the cationic lipid COATSOME® SS-EC (former name: SS-33/4PE-15; NOF Corporation, Tokyo, Japan), in accordance with the following formula

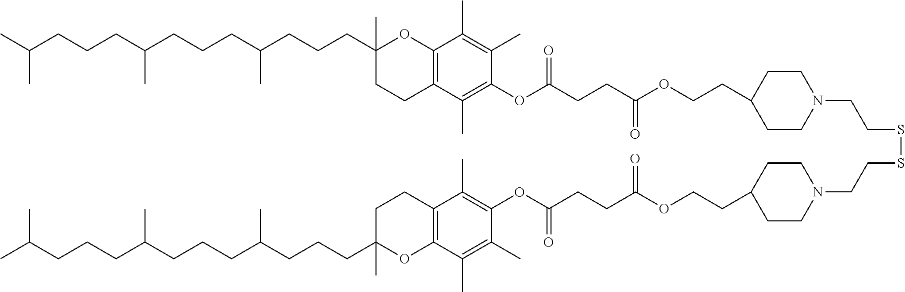 [SMILES: Cc1c(C)c2c(c(C)c1OC(=O)CCC(=O)OCCC1CCN(CCSSCCN3CCC(CCOC(=O)CCC(=O)Oc4c(C)c(C)c5c(c4C)CCC(C)(CCCC(C)CCCC(C)CCCC(C)C)O5)CC3)CC1)CCC(C)(CCCC(C)CCCC(C)CCCC(C)C)O2]

As described further below, those lipid nanoparticles are termed “GN01”.

In an embodiment, the lipid-based carriers encapsulating the RNA comprise a cationic lipid according to or derivable from the following formula (Heptadecan-9-yl 8-((2-hydroxyethyl)(6-oxo-6-(undecyloxy)hexyl)amino)octanoate):

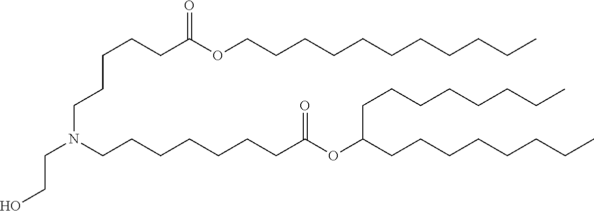 [SMILES: CCCCCCCCCCCOC(=O)CCCCCN(CCO)CCCCCCCC(=O)OC(CCCCCCCC)CCCCCCCC]

Other suitable (cationic or ionizable) lipids are disclosed in WO2009/086558, WO2009/127060, WO2010/048536, WO2010/054406, WO2010/088537, WO2010/129709, WO2011/153493, WO 2013/063468, US2011/0256175, US2012/0128760, US2012/0027803, U.S. Pat. No. 8,158,601, WO2016/118724, WO2016/118725, WO2017/070613, WO2017/070620, WO2017/099823, WO2012/040184, WO2011/153120, WO2011/149733, WO2011/090965, WO2011/043913, WO2011/022460, WO2012/061259, WO2012/054365, WO2012/044638, WO2010/080724, WO2010/21865, WO2008/103276, WO2013/086373, WO2013/086354, U.S. Pat. Nos. 7,893,302, 7,404,969, 8,283,333, 8,466,122 and 8,569,256 and US Patent Publication No. US2010/0036115, US2012/0202871, US2013/0064894, US2013/0129785, US2013/0150625, US2013/0178541, US2013/0225836, US2014/0039032 and WO2017/112865. In that context, the disclosures of WO2009/086558, WO2009/127060, WO2010/048536, WO2010/054406, WO2010/088537, WO2010/129709, WO2011/153493, WO 2013/063468, US2011/0256175, US2012/0128760, US2012/0027803, U.S. Pat. No. 8,158,601, WO2016/118724, WO2016/118725, WO2017/070613, WO2017/070620, WO2017/099823, WO2012/040184, WO2011/153120, WO2011/149733, WO2011/090965, WO2011/043913, WO2011/022460, WO2012/061259, WO2012/054365, WO2012/044638, WO2010/080724, WO2010/21865, WO2008/103276, WO2013/086373, WO2013/086354, U.S. Pat. Nos. 7,893,302, 7,404,969, 8,283,333, 8,466,122 and 8,569,256 and US Patent Publication No. US2010/0036115, US2012/0202871, US2013/0064894, US2013/0129785, US2013/0150625, US2013/0178541, US2013/0225836 and US2014/0039032 and WO2017/112865 specifically relating to (cationic) lipids suitable for LNPs (or liposomes, nanoliposomes, lipoplexes) are incorporated herewith by reference.

In embodiments, amino or cationic lipids as defined herein have at least one protonatable or deprotonatable group, such that the lipid is positively charged at a pH at or below physiological pH (e.g. pH 7.4), and neutral at a second pH, preferably at or above physiological pH. It will, of course, be understood that the addition or removal of protons as a function of pH is an equilibrium process, and that the reference to a charged or a neutral lipid refers to the nature of the predominant species and does not require that all of lipids have to be present in the charged or neutral form. Lipids having more than one protonatable or deprotonatable group, or which are zwitterionic, are not excluded and may likewise suitable in the context of the present invention. In some embodiments, the protonatable lipids have a pKa of the protonatable group in the range of about 4 to about 11, e.g., a pKa of about 5 to about 7.

The Lipid-based carriers encapsulating the RNA may comprise two or more (different) cationic lipids as defined herein.

In preferred embodiments, the lipid-based carriers encapsulating the RNA comprise at least one neutral lipid.

In embodiments, the lipid-based carriers encapsulating the RNA comprise a neutral lipid in a molar ratio of about 5% to about 25%, preferably in a molar ratio of about 8% to about 12%, for example in a molar ratio of about 9%, 9.5%, 10%, 10.5% or 11%. In preferred embodiments, the lipid-based carriers encapsulating the RNA comprise a neutral lipid in a molar ratio of about 10% (based on 100% total moles of lipids in the lipid-based carriers).

In embodiments, the lipid-based carriers encapsulating the RNA comprise the neutral lipid in a weight ratio of about 3% to about 20%, preferably in a weight ratio of about 9% to about 15%, for example in a weight ratio of about 10%, about 11%, about 12%, about 13%, about 14%. In embodiments, the lipid-based carriers encapsulating the RNA comprise the neutral lipid in a weight ratio of about 12.24% (based on 100% total weight of lipids in the lipid-based carriers).

In various embodiments, the molar ratio of the cationic lipid to the neutral lipid ranges from about 2:1 to about 8:1.

The term “neutral lipid” refers to any one of a number of lipid species that exist in either an uncharged or neutral zwitterionic form at physiological pH. Suitable neutral lipids include diacylphosphatidylcholines, diacylphosphatidylethanolamines, ceramides, sphingomyelins, dihydro sphingomyelins, cephalins, and cerebrosides.

In embodiments, the lipid-based carriers encapsulating the RNA comprises one or more neutral lipids, wherein the neutral lipid is selected from the group comprising distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoylphosphatidyethanol amine (SOPE), and 1,2-dielaidoyl-sn-glycero-3-phophoethanolamine (transDOPE), or mixtures thereof. In some embodiments, the lipid-based carriers encapsulating the RNA comprise a neutral lipid selected from DSPC, DPPC, DMPC, DOPC, POPC, DOPE and SM.

In preferred embodiments, the neutral lipid of the lipid-based carriers encapsulating the RNA is 1,2-distearoyl-sn-glycero-3-phosphocholine (DSPC).

In a specific embodiment, the lipid-based carriers encapsulating the RNA comprise a neutral lipid being resembled by the structure 1,2-diphytanoyl-sn-glycero-3-phosphoethanolamine (DPhyPE):

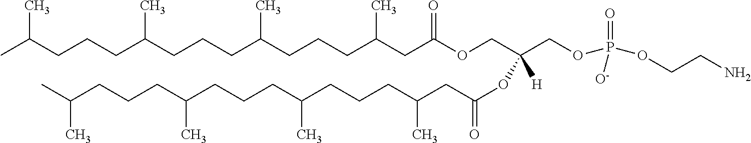 [SMILES: CC(C)CCCC(C)CCCC(C)CCCC(C)CC(=O)OC[C@H](COP(=O)([O-])OCCN)OC(=O)CC(C)CCCC(C)CCCC(C)CCCC(C)C]

In preferred embodiments, the lipid-based carriers encapsulating the RNA comprise at least one steroid or steroid analog.

In embodiments, the lipid-based carriers encapsulating the RNA comprises a steroid or steroid analog in a molar ratio of about 25% to about 55%, preferably in a molar ratio of about 33% to about 49%, for example in a molar ratio of about 38%, 39%, 40%, 41%, 42%, 43%, or about 44%. In preferred embodiments, the lipid-based carriers encapsulating the RNA comprise a steroid or steroid analog in a molar ratio of about 40.9% (based on 100% total moles of lipids in the carriers).

In embodiments, the lipid-based carriers encapsulating the RNA comprise the steroid or steroid analogue in a weight ratio of about 6% to about 40%, preferably in a weight ratio of about 18% to about 30%, for example in a weight ratio of about 21%, about 22%, about 23%, about 24%, about 25%, about 26%, or about 27%. In preferred embodiments, the lipid-based carriers encapsulating the RNA comprise a steroid or steroid analogue in a weight ratio of about 24.51% (based on 100% total weight of lipids in the lipid-based carriers).

Suitably, the molar ratio of the cationic lipid to steroid or steroid analogue may be in the range from about 2:1 to about 1:1.

In embodiments, the steroid or steroid analog of the lipid-based carriers encapsulating the RNA is cholesterol.

In some embodiments, the cholesterol may be a polymer conjugated cholesterol or a PEGylated cholesterol.

In embodiments, the lipid-based carriers encapsulating the RNA, preferably the LNPs, comprise: (a) the RNA, (b) a cationic lipid, (c) the aggregation reducing lipid (such as a PEG-conjugated lipid), (d) optionally, a non-cationic lipid (such as a neutral lipid), and (e) optionally, a steroid or steroid analog.

In some embodiments, the cationic lipids (as defined above), non-cationic lipids (as defined above), cholesterol (as defined above), and/or aggregation reducing lipid (as defined above) may be combined at various relative molar ratios. For example, the ratio of cationic lipid to non-cationic lipid to cholesterol-based lipid to aggregation reducing lipid (e.g. PEG-conjugated lipid) may be between about 30-60:20-35:20-30:1-15, or at a ratio of about 40:30:25:5, 50:25:20:5, 50:27:20:3, 40:30:20:10, 40:32:20:8, 40:32:25:3 or 40:33:25:2, or at a ratio of about 50:25:20:5, 50:20:25:5, 50:27:20:3 40:30:20:10, 40:30:25:5 or 40:32:20:8, 40:32:25:3 or 40:33:25:2, respectively.

In preferred embodiments, the lipid-based carriers encapsulating the RNA comprise a lipid of formula (III), at least one RNA as defined herein, a neutral lipid, a steroid and a Polymer-conjugated lipid. In preferred embodiments, the lipid of formula (III) is lipid compound 111-3, the neutral lipid is DSPC, the steroid is cholesterol, and the PEG-conjugated lipid is the compound of formula (IVa).

In particularly preferred embodiments, the lipid-based carriers encapsulating the RNA comprises
(i) at least one cationic lipid as defined herein, preferably a lipid of formula (III), more preferably lipid 111-3;
(ii) at least one neutral lipid as defined herein, preferably 1,2-distearoyl-sn-glycero-3-phosphocholine (DSPC);
(iii) at least one steroid or steroid analog as defined herein, preferably cholesterol; and
(iv) at least one aggregation reducing lipid, preferably a polymer-conjugated lipid, more preferably a PEG-conjugated lipid derived from formula (IVa).

In preferred embodiments, the lipid-based carriers encapsulating the RNA comprise (i) to (iv) in a molar ratio of about 20-60% cationic lipid, about 5-25% neutral lipid, about 25-55% steroid or steroid analogue, and about 0.5-15% aggregation reducing lipid, e.g. polymer conjugated lipid.

In specific embodiments, the lipid-based carriers encapsulating the RNA comprise or consist (i) to (iv) in a molar ratio of about 47.4% cationic lipid, about 10% neutral lipid, about 40.9% steroid or steroid analogue, and about 1.7% aggregation reducing lipid, e.g. polymer conjugated lipid.

In preferred embodiments, the lipid-based carriers encapsulating the RNA comprise (i) to (iv) in a weight ratio of about 30-70% cationic lipid, about 5-25% neutral lipid, about 10-40% steroid or steroid analogue, and about 2-20% aggregation reducing lipid, e.g. polymer conjugated lipid.

In specific embodiments, the lipid-based carriers encapsulating the RNA comprise or consist (i) to (iv) in a weight ratio of about 56.28% cationic lipid, about 12.24% neutral lipid, about 24.51% steroid or steroid analogue, and about 6.97% aggregation reducing lipid.

In embodiments, the composition comprises the lipid-based carriers encapsulating the RNA which have a molar ratio of approximately 50:10:38.5:1.5, preferably 47.5:10:40.8:1.7 or more preferably 47.4:10:40.9:1.7 (i.e. proportion (mol %) of cationic lipid (preferably lipid III-3), DSPC, cholesterol, and aggregation reducing lipid (e.g. polymer conjugated lipid, preferably PEG-lipid (preferably PEG-lipid of formula (IVa) with n=49 or with n=45))).

In embodiments, the composition comprises lipid-based carriers encapsulating the RNA which have a weight ratio of approximately 56:12:25:7, preferably 56.3:12.2:24.5:7 or more preferably 56.28:12.24:24.51:6.97 (i.e. proportion (weight %) of cationic lipid (preferably lipid 111-3), DSPC, cholesterol and aggregation reducing lipid (e.g. polymer conjugated lipid, preferably PEG-lipid (preferably PEG-lipid of formula (IVa) with n=49 or with n=45))).

In a specific embodiment, the lipid-based carriers encapsulating the RNA is a GNO1 lipid nanoparticle comprising a cationic lipid SS-EC, a neutral lipid 1,2-diphytanoyl-sn-glycero-3-phosphoethanolamine (DPhyPE), cholesterol, and a polymer conjugated lipid 1,2-dimyristoyl-rac-glycero-3-methoxypolyethylene glycol (PEG-DMG).

In embodiments, the GN01 lipid nanoparticles comprise:
(a) cationic lipid SS-EC (former name: SS-33/4PE-15; NOF Corporation, Tokyo, Japan) at an amount of 45-65 mol %;
(b) cholesterol at an amount of 25-45 mol %;
(c) DPhyPE at an amount of 8-12 mol %; and
(d) PEG-DMG 2000 at an amount of 1-3 mol %;
each amount being relative to the total molar amount of all lipidic excipients of the GN01 lipid nanoparticles.

In a preferred embodiment, the GN01 lipid nanoparticles comprises 59 mol % cationic lipid, 10 mol % neutral lipid, 29.3 mol % steroid and 1.7 mol % polymer conjugated lipid, preferably pegylated lipid. In a most preferred embodiment, the GNO1 lipid nanoparticles comprise 59 mol % cationic lipid SS-EC, 10 mol % DPhyPE, 29.3 mol % cholesterol and 1.7 mol % DMG-PEG 2000.

In a preferred embodiment, the GN01 lipid nanoparticles comprise 59 mol % cationic lipid COATSOME® SS-EC (former name: SS-33/4PE-15 as apparent from the examples section; NOF Corporation, Tokyo, Japan), 29.3 mol % cholesterol as steroid, 10 mol % DPhyPE as neutral lipid/phospholipid and 1.7 mol % DMG-PEG 2000 as polymer conjugated lipid. For GN01 lipid nanoparticles, N/P (lipid to nucleic acid, e.g. RNA mol ratio) preferably is 14 and total lipid/RNA mass ratio preferably is 40 (m/m).

In other embodiments, the lipid-based carriers encapsulating the RNA comprise the cationic lipid DLin-KC2-DMA (50 mol %) or DLin-MC3-DMA (50 mol %), the neutral lipid DSPC (10 mol %), the aggregation reducing lipid PEG-DOMG (1.5 mol %) and the structural lipid is cholesterol (38.5 mol %).

In other embodiments, the lipid-based carriers encapsulating the RNA comprises the cationic/ionizable lipid Heptadecan-9-yl 8-((2-hydroxyethyl)(6-oxo-6-(undecyloxy)hexyl)amino)octanoate, the neutral lipid 1,2-distearoyl-sn-glycero-3 phosphocholine (DSPC), the aggregation reducing lipid 1 monomethoxypolyethyleneglycol-2,3-dimyristylglycerol with polyethylene glycol of average molecular weight 2000 (PEG2000-DMG), and cholesterol, preferably at mol % 50/10/1.5/38.5.

In other embodiments, the lipid-based carrier encapsulating the RNA may be selected from any lipid-based carrier as described in WO2019/222424, WO2019/226925, WO2019/232095, WO2019/232097, or WO2019/232208, the disclosure of WO2019/222424, WO2019/226925, WO2019/232095, WO2019/232097, or WO2019/232208 relating to lipid-based carriers herewith incorporated by reference.

In other embodiments, the lipid-based carrier encapsulating the RNA may be composed of three lipid components, preferably imidazole cholesterol ester (ICE), 1,2-dioleoyl-sn-glycero-3-phosphoethanolamine (DOPE), and the aggregation reducing lipid 1,2-dimyristoyl-sn-glycerol, methoxypolyethylene glycol (DMG-PEG-2K).

In embodiments, the lipid-based carriers encapsulating the RNA of the pharmaceutical composition for multidose administration is a purified lipid-based carrier. Accordingly, the lipid-based carriers encapsulating the RNA have been purified by at least one purification step. Such a purification step may suitably selected from at least one step of tangential flow filtration and/or at least one step of clarification and/or at least one step of filtration.

The term “purified lipid-based carrier” as used herein has to be understood as lipid-based carriers encapsulating the RNA which have a higher purity after certain purification steps (e.g. tangential flow filtration, clarification filtration, chromatography steps) as compared to the starting material. Typical impurities that are essentially not present in the purified lipid-based carriers encapsulating the RNA comprise e.g. free lipids, organic solvents, empty lipid-based carriers (without RNA cargo), fused lipid-based carriers (lipid-based carriers exceeding the desired size), small micelles (lipid-based carriers that are smaller than the desired size), lipid-based carriers that do not comprise the desired components (e.g. lacking the aggregation reducing lipid), lipid degradation products etc. Other potential impurities may be derived from the synthesis of the individual lipid components. Accordingly, the lipid components used in formulating the lipid-based carriers encapsulating the RNA have a purity level of at least 80%, preferably at least 90%, more preferably at least 95%. It is desirable for the “degree of lipid-based carrier purity” to be as close as possible to 100%. “Purified lipid-based carriers” as used herein have a degree of purity of more than 75%, 80%, 85%, very particularly 90%, 91%, 92%, 93%, 94%, 95%, 96%, 97%, 98% and most favorably 99% or more. The degree of purity may for example be determined by an analytical HPLC (to determine contaminations and to determine the lipid ratio in the carrier) or by determining the size and size distribution of the obtained lipid-based carriers (e.g. using DLS, NTA, MFI) or the shape of the lipid carriers (e.g. by EM analysis).

In various embodiments, the pharmaceutical composition for multidose administration has a certain clarity, e.g. without showing signs of increased turbidity. The detection of a certain turbidity may be an indicator for agglomeration of lipid-based carriers or precipitation of lipid-based carriers. Accordingly the pharmaceutical composition for multidose administration having the desired quality is typically clear. An reduced quality of the composition is visible, for example, as turbidity within the composition, wherein increasing turbidity may be correlated with decreasing product quality and decreasing stability, which may eventually result in the formation of precipitates. An increase of turbidity may be caused by e.g. the addition of an antimicrobial preservative that is not compatible with the lipid-based carriers encapsulating an RNA.

Turbidity is the measure of relative clarity of a liquid. It is an optical characteristic of a liquid composition that is a measurement of the amount of light that is scattered by material in the water when a light is shined through the water sample. The higher the intensity of scattered light, the higher the turbidity. Turbidity may be measured at 860 nm with a detecting angle of 900 using commercially available instruments and methods known in the art. An example for a commercially available instrument is a NEPHLA turbidimeter, available from Dr. Lange, Dusseldorf, Germany. The system is calibrated with formazin as standard and the results were given in formazin nephelometric units (FNU).

In embodiments, the pharmaceutical composition for multidose administration comprising the lipid-based carriers encapsulating the RNA has a turbidity ranging from about 150 FNU to about 0.0 FNU. In embodiments, the composition has a turbidity of about 100 FNU or less, preferably of about 50 FNU or less, more preferably of about 25 FNU or less.

In embodiments, the pharmaceutical composition for multidose administration comprising the lipid-based carriers encapsulating the RNA has an RNA concentration of 1 g/l and a turbidity of about 100 FNU or less, preferably of about 50 FNU or less, more preferably of about 25 FNU or less. In embodiments, the composition comprising the lipid-based carriers encapsulating the RNA has an RNA concentration of 0.5 g/l and a turbidity of about 100 FNU or less, preferably of about 50 FNU or less, more preferably of about 25 FNU or less.

In embodiments, the pharmaceutical composition for multidose administration comprises a buffer e.g. comprising a sugar and/or a salt and/or a buffering agent.

In embodiments, the pharmaceutical composition for multidose administration comprising the lipid-based carriers encapsulating the RNA further comprises a sugar, preferably a disaccharide. In embodiments, the concentration of the sugar comprised in the composition is in a range from about 5 mM to about 300 mM. In embodiments, the sugar comprised in the composition is sucrose, preferably in a concentration of about 14 mM. In embodiments where a sugar alcohol is comprised as an antimicrobial preservative (e.g. Xylitol), the concentration of sucrose may be reduced, e.g. may be lower than 14 mM.

In embodiments, the pharmaceutical composition for multidose administration comprising the lipid-based carriers encapsulating the RNA further comprises a salt, preferably NaCl. In embodiments, the concentration of the salt comprised in the composition is in a range from about 10 mM to about 300 mM, preferably about 150 mM. In embodiments, the salt comprised in composition is NaCl, preferably in a concentration of about 150 mM.

In embodiments, the composition comprising the lipid-based carriers encapsulating the RNA comprises a buffering agent, preferably selected from Tris, HEPES, NaPO4 or combinations thereof. In embodiments, the buffering agent is in a concentration ranging from about 0.1 mM to about 100 mM. In embodiments, the buffering agent is NaPO4, preferably in a concentration of about 1 mM. In other embodiments, the buffering agent is Tris.

In embodiments, the composition has a pH in a range of about pH 7.0 to about pH 8.0. In preferred embodiments, the composition has a pH of about pH 7.4.

In other embodiments, the composition has a pH in a range of about pH 6.0. Such a pH may further improve the antimicrobial effect of the antimicrobial preservative (e.g. the preferred aromatic alcohol).

In embodiments, the composition has an osmolality of about 250 mOsmol/kg to about 450 mOsmol/kg, preferably of about 335 mOsmol/kg. The Osmolality of the composition may be determined by the skilled person using an osmometer.

In preferred embodiments, the pharmaceutical composition for multidose administration is free of virus particles e.g. attenuated viruses or virus fragments.

In preferred embodiments, the pharmaceutical composition for multidose administration is essentially free of peptide or proteins (e.g. peptide or protein antigens).

In preferred embodiments, the pharmaceutical composition for multidose administration does not comprise and additionally added adjuvant. Accordingly, the pharmaceutical composition for multidose administration comprises lipid-based carriers encapsulating an RNA and lacks an additional adjuvant component.

The term “adjuvant” is for example intended to refer to a pharmacological and/or immunological agent that may modify, e.g. enhance, the effect of other agents or that may be suitable to support administration and delivery of the composition. The term “adjuvant” refers to a broad spectrum of substances. Typically, these substances are able to increase the immunogenicity of antigens. For example, adjuvants may be recognized by the innate immune systems and, e.g., may elicit an innate immune response (that is, a non-specific immune response). “Adjuvants” typically do not elicit an adaptive immune response.

In preferred embodiments, the pharmaceutical composition for multidose administration is stable for at least 6 hours, preferably for at least about 1 day. In preferred embodiments, the pharmaceutical composition for multidose administration is stable for at least 6 hours to about 6 months. In embodiments, the pharmaceutical composition for multidose administration is stable for at least 1 day, 2 days, 3 days, 4 days, 5 days, 6 days, 7 days, 2 weeks, 3 weeks, 4 weeks, 1 month, 2 months, 3 months, 4 months, 5 months, or 6 months.

In preferred embodiments, the pharmaceutical composition for multidose administration is stable at a temperature of about 5° C. to about 25° C. In embodiments, the pharmaceutical composition for multidose administration is stable at a temperature of about 5° C., 6° C., 7° C., 8° C., 9° C., 10° C., 15° C., 20° C., or 25° C.

As used herein, “stable” refers to a composition comprising lipid-based carriers encapsulating an RNA where the measured values for various physiochemical parameters are within a defined range after storage. In one embodiment, the composition comprising lipid-based carriers encapsulating an RNA is analyzed to assess stability according to various parameters. Suitable stability parameters include, without limitation, RNA integrity, Z-average particle size, polydispersity index (PDI), the amount of free RNA in the composition, encapsulation efficiency of the RNA (proportion of the RNA in percent incorporated with lipid-based carriers), shape and morphology of the lipid-based carriers encapsulating an RNA, pH, osmolality, or turbidity. Further, “stable” refers to a composition comprising lipid-based carriers encapsulating an RNA where the measured values for various functional parameters are within a defined range after storage. In one embodiment, the composition comprising lipid-based carriers encapsulating an RNA is analyzed to assess the potency of the composition including for example the expression of the encoded peptide or protein, the induction of specific antibody titers, the induction of neutralizing antibody titers, the induction of T-cell, the reactogenicity of the composition including for example the induction of innate immune responses etc. In preferred embodiments, the term “stable” refers to RNA integrity.

The pharmaceutical composition for multidose administration may be obtained by combining a pharmaceutical composition comprising lipid-based carriers encapsulating an RNA with at least one antimicrobial preservative selected from at least one aromatic alcohol, at least one sugar alcohol, thiomersal, preferably at least one aromatic alcohol. The obtained pharmaceutical composition comprising lipid-based carriers encapsulating an RNA and the at least one antimicrobial preservative may readily be used for multidose administration.

Advantageously, the physiochemical properties of the RNA and/or the lipid-based carriers are stable in the presence of the at least one antimicrobial preservative, e.g. in the presence of at least one aromatic alcohol, at least one sugar alcohol, or thiomersal. In particular, the physiochemical properties of the RNA and/or the lipid-based carriers are stable in the presence of at least one aromatic alcohol, e.g. in the presence of phenoxyethanol or benzyl alcohol.

Accordingly, the composition is stable for at least about 1 day after a first dose withdrawal and/or after formulation. Preferably, the composition is stable for at least 6 hours, preferably for at least 1 day after formulation of the composition. In preferred embodiments, the pharmaceutical composition for multidose administration is stable for at least 6 hours to about 6 months after formulation of the composition. In embodiments, the pharmaceutical composition for multidose administration is stable for at least 1 day, 2 days, 3 days, 4 days, 5 days, 6 days, 7 days, 2 weeks, 3 weeks, 4 weeks, 1 month, 2 months, 3 months, 4 months, 5 months, or 6 months after formulation of the composition.

In preferred embodiments, the pharmaceutical composition for multidose administration is stable after formulation of the composition at a temperature of about 5° C. to about 25° C. In embodiments, the pharmaceutical composition for multidose administration is stable after formulation of the composition at a temperature of about 5° C., 6° C., 7° C., 8° C., 9° C., 10° C., 15° C., 20° C., or 25° C.

The pharmaceutical composition is configured for multidose administration. Accordingly, the physiochemical properties of the RNA and/or the lipid-based carriers are stable after a first administration or withdrawal of a dose.

Preferably, the composition is stable for at least 6 hours, preferably for at least 1 day after a first dose withdrawal. In preferred embodiments, the pharmaceutical composition for multidose administration is stable for at least 6 hours to about 6 months after a first dose withdrawal. In embodiments, the pharmaceutical composition for multidose administration is stable for at least 1 day, 2 days, 3 days, 4 days, 5 days, 6 days, 7 days, 2 weeks, 3 weeks, 4 weeks, 1 month, 2 months, 3 months, 4 months, 5 months, or 6 months after a first dose withdrawal.

In preferred embodiments, the pharmaceutical composition for multidose administration is stable after a first dose withdrawal at a temperature of about 5° C. to about 25° C. In embodiments, the pharmaceutical composition for multidose administration is stable after a first dose withdrawal at a temperature of about 5° C., 6° C., 7° C., 8° C., 9° C., 10° C., 15° C., 20° C., or 25° C.

In preferred embodiments, after formulation or after a first dose withdrawal, the integrity of the RNA decreases less than about 30%, preferably less than about 20%, more preferably less than about 10%. RNA integrity is suitably determined using analytical HPLC, preferably analytical RP-HPLC. In preferred embodiments, after formulation or after a first dose withdrawal, the RNA has an RNA integrity ranging from about 40% to about 100%. RNA integrity is suitably determined using analytical HPLC, preferably analytical RP-HPLC.

In particularly preferred embodiments, after formulation or after a first dose withdrawal, the integrity of the RNA decreases less than about 30%, preferably less than about 20%, more preferably less than about 10% in comparison to a reference composition that does not comprise the antimicrobial preservative (e.g. aromatic alcohol, preferably Phenoxyethanol).

In the context of the invention, a reference composition relates to a composition with the same properties (e.g. same RNA sequence, same lipid formulation) but without an antimicrobial preservative.

Accordingly, in preferred embodiments, the physiochemical properties of the lipid-based carriers encapsulating an mRNA are stable (in the presence of the antimicrobial preservative (e.g. aromatic alcohol, preferably Phenoxyethanol)) in comparison to a reference composition that does not comprise the respective antimicrobial preservative.

In preferred embodiments, after formulation or after a first dose withdrawal, the amount of free RNA does not increase by more than 20%, preferably by not more than 10%, more preferably by not more than 5%. In embodiments, after formulation or after a first dose withdrawal, the amount of free RNA in the composition ranges from about 30% to about 0%. Free RNA is suitably determined using a RiboGreen assay.

In particularly preferred embodiments, after formulation or after a first dose withdrawal, the amount of free RNA does not increase by more than 20%, preferably by not more than 10%, more preferably by not more than 5% in comparison to a reference composition that does not comprise the antimicrobial preservative (e.g. aromatic alcohol preferably Phenoxyethanol).

In embodiments, after formulation or after a first dose withdrawal, the percentage of RNA encapsulation does not decrease by more than 20%, preferably by not more than 10%. In embodiments, after formulation or after a first dose withdrawal, the percentage of RNA encapsulation ranges from about 60% to about 100%. RNA encapsulation is suitably determined using a RiboGreen assay.

In preferred embodiments, after formulation or after a first dose withdrawal, the percentage of RNA encapsulation does not decrease by more than 20%, preferably by not more than 10% in comparison to a reference composition that does not comprise the antimicrobial preservative (e.g. aromatic alcohol preferably Phenoxyethanol).

In embodiments, after formulation or after a first dose withdrawal, the PDI value does not increase by more than a value of about 0.2, preferably by not more than a value of about 0.1. In embodiments, after formulation or after a first dose withdrawal, the PDI value ranges from about 0.4 to about 0.0. PDI is suitably determined using DLS.

In embodiments, after formulation or after a first dose withdrawal, the PDI value does not increase by more than a value of about 0.2, preferably by not more than a value of about 0.1 in comparison to a reference composition that does not comprise the antimicrobial preservative (e.g. aromatic alcohol preferably Phenoxyethanol).

In embodiments, after formulation or after a first dose withdrawal, the Z-average size of the lipid based carriers encapsulating the RNA does not increase by more than 20%, preferably by not more than 10%. In embodiments, after formulation or after a first dose withdrawal, the Z-average size of the lipid based carriers encapsulating the RNA ranges from about 50 nm to about 150 nm. Z-average size is suitably determined using DLS.

In preferred embodiments, after formulation or after a first dose withdrawal, the Z-average size of the lipid based carriers encapsulating the RNA does not increase by more than 20%, preferably by not more than 10% in comparison to a reference composition that does not comprise the antimicrobial preservative (e.g. aromatic alcohol preferably Phenoxyethanol).

In embodiments, after formulation or after a first dose withdrawal, the number of sub visible particles ≥2 μm (#per ml) is not increased by more than 20%, preferably by not more than 10%.

In preferred embodiments, after formulation or after a first dose withdrawal, the number of sub visible particles ≥2 μm (#per ml) is not increased by more than 20%, preferably by not more than 10% in comparison to a reference composition that does not comprise the antimicrobial preservative (e.g. aromatic alcohol preferably Phenoxyethanol).

In embodiments, after formulation or after a first dose withdrawal, the potency of the composition decreases less than about 30%, preferably less than 20%, more preferably less than 10%. In embodiments, potency is the expression of the encoded peptide or protein upon administration of the composition to a cell, and/or the induction of specific antibody titers upon administration of the composition to a cell, and/or the induction of neutralizing antibody titers upon administration of the composition to a cell, and/or the induction of antigen-specific T-cell responses upon administration of the composition to a cell.

In preferred embodiments, after formulation or after a first dose withdrawal, the potency of the composition decreases less than about 30%, preferably less than 20%, more preferably less than 10% in comparison to a reference composition that does not comprise the antimicrobial preservative (e.g. aromatic alcohol preferably Phenoxyethanol).

In embodiments, after formulation or after a first dose withdrawal, the reactogenicity of the composition does not increase by more than 20%, preferably by not more than 10%. In embodiments, reactogenicity may be the induction of (undesired) innate immune responses upon administration of the composition to a cell (e.g. cytokine induction).

In preferred embodiments, after formulation or after a first dose withdrawal, the reactogenicity of the composition does not increase by more than 20%, preferably by not more than 10% in comparison to a reference composition that does not comprise the antimicrobial preservative (e.g. aromatic alcohol preferably Phenoxyethanol).

Advantageously, the at least one antimicrobial preservative e.g. at least one aromatic alcohol, at least one sugar alcohol, or thiomersal, preferably the at least one aromatic alcohol, has an antimicrobial activity in the presence of lipid-based carriers encapsulating an RNA.

Accordingly, in particularly preferred embodiments, the pharmaceutical composition for multidose administration comprising lipid-based carriers encapsulating an RNA and at least one antimicrobial preservative selected from at least one aromatic alcohol, at least one sugar alcohol, thiomersal, or a combination thereof, is microbially preserved or microbially stable.

In particularly preferred embodiments, the pharmaceutical composition for multidose administration comprising lipid-based carriers encapsulating an RNA and at least one antimicrobial preservative selected from at least one aromatic alcohol is microbially preserved or microbially stable.

Microbially preserved or microbially stable in the context of the invention has to be understood as a certain protection against microbial contamination (e.g. bacterial contamination or fungal contamination) preliminary conferred by the at least one antimicrobial preservative (e.g. according to the invention, by at least one aromatic alcohol (e.g. Phenoxyethanol), at least one sugar alcohol (e.g. Xylitol), or thiomersal). Notably, a certain protection against microbial contamination may be provided as the composition of the invention has been produced under sterile conditions, e.g. under GMP or cGMP conditions. However, in a multidose use or multiuse setting, microbial contaminations may be introduced into the pharmaceutical composition of the invention (e.g. by withdrawing a first dose with a syringe). Therefore, the term “microbially preserved” may be understood as being at least bacteriostatic for at least 6 hours at a typical temperature of a multidose composition, e.g. 5° C. (fridge) or 25° C. (room temperature). The term “microbially preserved” may also be understood as being bactericide or fungicide.

The ability of antimicrobial preservatives to inhibit or kill microorganisms in pharmaceutical formulations may be evaluated using antimicrobial effectiveness tests (AETs) (see for example Moser, Cheryl L., and Brian K. Meyer. “Comparison of compendial antimicrobial effectiveness tests: a review.” Aaps Pharmscitech 12.1 (2011): 222-226). Procedures for determining antimicrobial preservation of a pharmaceutical compositions may be conducted by a test performed according to the European Pharmacopeia Ph Eur 5.1. 3 (Efficacy of Antimicrobial Preservation) and/or USP (Antimicrobial Effectiveness Testing).

In preferred embodiments, the pharmaceutical composition for multidose administration is microbially preserved for at least 6 hours, preferably for at least about 1 day. In preferred embodiments, the pharmaceutical composition for multidose administration is microbially preserved for at least 6 hours to about 6 months. In embodiments, the pharmaceutical composition for multidose administration is microbially preserved for at least 1 day, 2 days, 3 days, 4 days, 5 days, 6 days, 7 days, 2 weeks, 3 weeks, 4 weeks, 1 month, 2 months, 3 months, 4 months, 5 months, or 6 months.

In preferred embodiments, the pharmaceutical composition for multidose administration is microbially preserved at a temperature of about 5° C. to about 25° C. In embodiments, the pharmaceutical composition for multidose administration is microbially preserved at a temperature of about 5° C., 6° C., 7° C., 8° C., 9° C., 10° C., 15° C., 20° C., or 25° C.

Preferably, the composition is microbially preserved for at least 6 hours, preferably for at least 1 day after a first dose withdrawal. In preferred embodiments, the pharmaceutical composition for multidose administration is microbially preserved for at least 6 hours to about 6 months after a first dose withdrawal. In embodiments, the pharmaceutical composition for multidose administration is microbially preserved for at least 1 day, 2 days, 3 days, 4 days, 5 days, 6 days, 7 days, 2 weeks, 3 weeks, 4 weeks, 1 month, 2 months, 3 months, 4 months, 5 months, or 6 months after a first dose withdrawal.

In preferred embodiments, the pharmaceutical composition for multidose administration is microbially preserved after a first dose withdrawal at a temperature of about 5° C. to about 25° C. In embodiments, the pharmaceutical composition for multidose administration is microbially preserved after a first dose withdrawal at a temperature of about 5° C., 6° C., 7° C., 8° C., 9° C., 10° C., 15° C., 20° C., or 25° C.

In preferred embodiments, the pharmaceutical composition for multidose administration is bacteriostatic for gram positive and/or gram negative bacteria. That bacteriostatic effect may be analyzed by performing a test according to the European Pharmacopeia Ph Eur 5.1.3. (Efficacy of Antimicrobial Preservation) and/or USP (Antimicrobial Effectiveness Testing).

In preferred embodiments, the pharmaceutical composition for multidose administration is bacteriostatic for Pseudomonas aeruginosa and/or Staphylococcus aureus. That bacteriostatic effect may be analyzed by performing a test according to the European Pharmacopeia Ph Eur 5.1.3. (Efficacy of Antimicrobial Preservation) and/or USP (Antimicrobial Effectiveness Testing).

In preferred embodiments, the pharmaceutical composition for multidose administration is bactericide for gram positive and/or gram negative bacteria. That bactericide effect may be analyzed by performing a test according to the European Pharmacopeia Ph Eur 5.1.3. (Efficacy of Antimicrobial Preservation) and/or USP (Antimicrobial Effectiveness Testing).

In preferred embodiments, the pharmaceutical composition for multidose administration is bactericide for Pseudomonas aeruginosa and/or Staphylococcus aureus. That bactericide effect may be analyzed by performing a test according to the European Pharmacopeia Ph Eur 5.1.3. (Efficacy of Antimicrobial Preservation) and/or USP (Antimicrobial Effectiveness Testing).

In preferred embodiments, the pharmaceutical composition for multidose administration is fungicide or fungistatic for Candida albicans and/or Aspergillus niger. That fungicide or fungistatic effect may be analyzed by performing a test according to the European Pharmacopeia Ph Eur 5.1.3. (Efficacy of Antimicrobial Preservation) and/or USP (Antimicrobial Effectiveness Testing).

Suitably, the pharmaceutical composition for multidose administration fulfils Category B requirements according to Ph. Eur. 5.1.3. For example, after inoculation of the pharmaceutical composition for multidose administration with a certain microorganism inoculum (e.g. 3×10\hat{ }5 cfu/ml Pseudomonas aeruginosa or Staphylococcus aureus), a log 1 reduction of the respective microorganism after 24 h is observed.

Suitably, the pharmaceutical composition for multidose administration fulfils Category A requirements according to Ph. Eur. 5.1.3. For example, after inoculation of the pharmaceutical composition for multidose administration with a certain microorganism inoculum (e.g. 3×10\hat{ }5 cfu/ml Pseudomonas aeruginosa or Staphylococcus aureus), a log 2 reduction of the respective microorganism after 6 h, and a log 3 reduction of the respective microorganism after 24 h is observed.

In preferred embodiments, the composition for multidose administration as described in the context of the first aspect is for parental use, preferably for multi-dose parenteral use. Preferably, the composition for multidose administration is a vaccine.

### Vaccine for Multidose Administration

In a second aspect, the present invention provides a vaccine for multidose administration comprising lipid-based carriers encapsulating an RNA, preferably mRNA, wherein the composition comprises at least one antimicrobial preservative selected from at least one aromatic alcohol, at least one sugar alcohol, thiomersal, or a combination thereof.

In a preferred embodiment, the vaccine provided herein is a multidose vaccine.

The term “multidose vaccine” as used herein refers to a vaccine that comprises more than one dose of an active pharmaceutical ingredient (API). In the context of the invention, the active pharmaceutical ingredient is an RNA, e.g. a therapeutic RNA, preferably mRNA. Accordingly, the vaccine for multidose administration suitably comprises more than one dose of RNA.

In preferred embodiments, the vaccine for multidose administration comprises or consists of the pharmaceutical composition for multidose administration as defined in the context of the first aspect.

It has to be noted that features and embodiments that are described in the context of the pharmaceutical composition for multidose administration of the first aspect may also be applicable to the vaccine for multidose administration of the second aspect. Likewise, features and embodiments that are described in the context of the vaccine for multidose administration of the second aspect may also be applicable to the pharmaceutical composition for multidose administration of the first aspect.

Suitably, the at least one antimicrobial preservative may be selected from bronopol, cetrimide, cetylpyridinium chloride, chlorhexidine, chlorobutanol, ethyl alcohol, hexetidine, imidurea, phenylmercuric nitrate, propylene glycol, an aromatic alcohol, a sugar alcohol, and/or thimerosal.

In preferred embodiments, the vaccine for multidose administration comprises at least one antimicrobial preservative selected from at least one aromatic alcohol, at least one sugar alcohol, thiomersal.

In particularly preferred embodiments, the vaccine for multidose administration comprises at least one antimicrobial preservative selected from at least one aromatic alcohol, wherein the at least one aromatic alcohol may be selected from phenylethyl alcohol, phenoxyethanol, benzyl alcohol, or a combination thereof.

In preferred embodiments, the vaccine for multidose administration comprises at least one antimicrobial preservative selected from at least one aromatic alcohol, wherein the at least one aromatic alcohol is phenoxyethanol.

In preferred embodiments, the vaccine for multidose administration comprises at least one antimicrobial preservative selected from at least one aromatic alcohol, wherein the at least one aromatic alcohol is in a concentration of 0.1% (w/v) to 2% (w/v), preferably 0.5% (w/v).

In preferred embodiments, the at least one aromatic alcohol (e.g. phenoxyethanol) is in a concentration of about 0.5% (w/v) to about 1% (w/v).

In preferred embodiments, the vaccine for multidose administration comprises at least one antimicrobial preservative selected from at least one aromatic alcohol, wherein the at least one aromatic alcohol is phenoxyethanol, wherein phenoxyethanol is in a concentration of 0.1% (w/v) to 2% (w/v), preferably 0.5% (w/v).

In embodiments, the vaccine for multidose administration comprises at least one antimicrobial preservative selected from thiomersal in a concentration of 0.0005% (w/v) to 0.05% (w/v).

In preferred embodiments, the vaccine for multidose administration comprises at least one antimicrobial preservative selected from at least one sugar alcohol, wherein the at least one sugar alcohol may be selected from xylitol, sorbitol, and/or glycerol, or a combination thereof.

In preferred embodiments, the vaccine for multidose administration comprises at least one antimicrobial preservative selected from at least one sugar alcohol, wherein the at least one sugar alcohol is xylitol.

In preferred embodiments, the vaccine for multidose administration comprises at least one antimicrobial preservative selected from at least one sugar alcohol, wherein the at least one sugar alcohol is in a concentration of about 10 mM to about 200 mM, more preferably in a concentration of about 25 mM to about 200 mM, even more preferably in a concentration of about 25 mM to about 150 mM In preferred embodiments, the vaccine for multidose administration comprising lipid-based carriers encapsulating an RNA may comprise more than one, preferably 2, 3, 4, 5, 6 or more of the antimicrobial preservatives as defined in the context of the first aspect (e.g., 2, 3, 4, 5, 6 or more of phenylethyl alcohol, phenoxyethanol, benzyl alcohol, thiomersal, xylitol, sorbitol, and/or glycerol).

In preferred embodiments, the vaccine for multidose administration comprises at least two antimicrobial preservatives selected from at least one aromatic alcohol and from at least one sugar alcohol.

In embodiments where at least one aromatic alcohol (e.g. phenoxyethanol) and at least one sugar alcohol (e.g. xylitol) are used as antimicrobial preservatives, the at least one aromatic alcohol (e.g. phenoxyethanol) is in a concentration of about 0.1% (w/v) to about 5% (w/v), preferably in a concentration of about 0.1% (w/v) to about 2%, and the at least one sugar alcohol (e.g. xylitol) is in a concentration of about 10 mM to about 500 mM, preferably in a concentration of about 10 mM to about 200 mM, more preferably in a concentration of about 25 mM to about 200 mM, even more preferably in a concentration of about 25 mM to about 150 mM In preferred embodiments, the vaccine for multidose administration comprises phenoxyethanol and xylitol.

In preferred embodiments of the second aspect, the vaccine for multidose administration comprises phenoxyethanol and xylitol, wherein phenoxyethanol is in a concentration of about 0.1% (w/v) to about 2%, preferably 0.5% (w/v), and wherein xylitol is in a concentration of about 10 mM to about 200 mM, preferably about 25 mM to about 150 mM.

In preferred embodiments, the vaccine for multidose administration comprises an RNA, preferably mRNA, as defined in the context of the first aspect, wherein the RNA is encapsulated in a lipid-based carrier as defined in the context of the first aspect.

In preferred embodiments, the RNA of the vaccine for multidose administration encodes at least one antigenic peptide or protein selected from or derived from a pathogen, preferably as defined in the context of the first aspect. Such pathogens may be bacterial, viral, or protozoological (multicellular) pathogenic organisms. Preferably, the pathogen evokes an immunological reaction or an infection in a subject, in particular a mammalian subject, preferably a human subject.

In embodiments, the vaccine for multidose administration is against a pathogen, for example against a virus, against a bacterium, or against a protozoan.

In embodiments, the vaccine for multidose administration is against at least one pathogen selected from List 1.

In embodiments, the vaccine for multidose administration is against a virus.

In preferred embodiments, the vaccine for multidose administration is against a (pandemic) coronavirus (e.g. SARS-CoV-2 coronavirus vaccine).

In particularly preferred embodiments, the vaccine for multidose administration comprises an RNA encoding an antigen or epitope as defined herein, preferably selected or derived from a SARS-CoV-2 virus. The RNA encoding the antigen or epitope is encapsulated in a lipid-based carrier, preferably LNPs, comprising
(i) at least one cationic lipid, preferably a lipid of formula (III), more preferably lipid 111-3;
(ii) at least one neutral lipid, preferably 1,2-distearoyl-sn-glycero-3-phosphocholine (DSPC);
(iii) at least one steroid or steroid analog, preferably 1,2-distearoyl-sn-glycero-3-phosphocholine (DSPC); and
(iv) at least one aggregation reducing lipid, preferably a PEG-conjugated lipid derived from formula (IVa); and
wherein (i) to (iv) are in a molar ratio of about 47.4% cationic lipid, 10% neutral lipid, 40.9% steroid or steroid analog, and 1.7% aggregation reducing lipid. The N/P ratio of the lipid-based carriers to the is in a range from about 1 to about 10, preferably in a range from about 5 to about 7, more preferably about 6. In preferred embodiments, the vaccine for multidose administration comprises an aromatic alcohol (e.g. Phenoxyethanol), preferably in a concentration of 0.5% (w/v) and, optionally, at least one sugar alcohol, preferably xylitol.

In particularly preferred embodiments, the Coronavirus vaccine for multidose administration comprises an RNA encoding an antigen or epitope selected or derived from a SARS-CoV-2 virus, wherein the RNA comprises or consists of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 3 or a fragment or variant of that sequence. Preferably, the RNA encoding the antigen or epitope selected or derived from a SARS-CoV-2 virus does not comprise chemically modified nucleotides. The RNA encoding the antigen or epitope selected or derived from a SARS-CoV-2 virus is encapsulated in a lipid-based carrier, preferably LNPs, comprising
(i) at least one cationic lipid, preferably a lipid of formula (III), more preferably lipid 111-3;
(ii) at least one neutral lipid, preferably 1,2-distearoyl-sn-glycero-3-phosphocholine (DSPC);
(iii) at least one steroid or steroid analog, preferably 1,2-distearoyl-sn-glycero-3-phosphocholine (DSPC); and
(iv) at least one aggregation reducing lipid, preferably a PEG-conjugated lipid derived from formula (IVa); and
wherein (i) to (iv) are in a molar ratio of about 47.4% cationic lipid, 10% neutral lipid, 40.9% steroid or steroid analog, and 1.7% aggregation reducing lipid. The N/P ratio of the lipid-based carriers to the RNA in the Coronavirus vaccine for multidose administration is in a range from about 1 to about 10, preferably in a range from about 5 to about 7, more preferably about 6. In preferred embodiments, the Coronavirus vaccine for multidose administration comprises phenoxyethanol, preferably in a concentration of 0.5% (w/v) and, optionally, at least one sugar alcohol, preferably xylitol.

In preferred embodiments, the vaccine for multidose administration is against a is against a Rabies virus.

In particularly preferred embodiments, the Rabies vaccine for multidose administration comprises an RNA encoding an antigen or epitope selected or derived from a Rabies virus, wherein the RNA comprises or consists of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 6 or a fragment or variant of any of these sequences. Preferably, the RNA encoding the antigen or epitope selected or derived from a Rabies virus does not comprise chemically modified nucleotides. The RNA encoding the antigen or epitope selected or derived from a Rabies virus is encapsulated in a lipid-based carrier, preferably LNPs, comprising
(i) at least one cationic lipid, preferably a lipid of formula (III), more preferably lipid II-3;
(ii) at least one neutral lipid, preferably 1,2-distearoyl-sn-glycero-3-phosphocholine (DSPC);
(iii) at least one steroid or steroid analog, preferably 1,2-distearoyl-sn-glycero-3-phosphocholine (DSPC); and
(iv) at least one aggregation reducing lipid, preferably a PEG-conjugated lipid derived from formula (IVa); and
wherein (i) to (iv) are in a molar ratio of about 47.4% cationic lipid, 10% neutral lipid, 40.9% steroid or steroid analog, and 1.7% aggregation reducing lipid. The N/P ratio of the lipid-based carriers to the RNA in the Rabies vaccine for multidose administration is in a range from about 1 to about 10, preferably in a range from about 5 to about 7, more preferably about 6. In preferred embodiments, the Rabies vaccine for multidose administration comprises phenoxyethanol, preferably in a concentration of 0.5% (w/v) and, optionally, at least one sugar alcohol, preferably xylitol.

The vaccine for multidose administration of the second aspect typically comprises a safe and effective amount of the lipid-based carriers encapsulating an RNA as defined in the first aspect. As used herein, “safe and effective amount” means an amount of the lipid-based carriers encapsulating the RNA that significantly induces a positive modification of a disease or disorder related to an infection with a pathogen (e.g. a virus, a bacterium, a protozoan) as specified herein. At the same time, a “safe and effective amount” is small enough to avoid serious side-effects. In relation to the vaccine of composition for multidose administration, the expression “safe and effective amount” may preferably mean an amount of the composition or vaccine that is suitable for stimulating the adaptive immune system against a pathogen as specified herein in such a manner that no excessive or damaging immune reactions are achieved.

A “safe and effective amount” of the composition or vaccine as defined herein will vary in connection with the particular condition to be treated and also with the age and physical condition of the patient to be treated, the severity of the condition, the duration of the treatment, the nature of the accompanying therapy, of the particular pharmaceutically acceptable carrier used, and similar factors, within the knowledge and experience of the skilled person. Moreover, the “safe and effective amount” of the composition of the first aspect, or the vaccine of the second aspect may depend from application/delivery route (intradermal, intramuscular, intranasal), application device (jet injection, needle injection, microneedle patch, electroporation device) and/or complexation/formulation. Moreover, the “safe and effective amount” of the composition of the first aspect, or the vaccine of the second aspect may depend from the physical condition of the treated subject (infant, pregnant women, immunocompromised human subject etc.).

The term “safe and effective amount” may also be understood as “dose” or “effective dose”.

In embodiments, the vaccine for multidose administration is preferably administered locally. Routes for local administration in general include, for example, topical administration routes but also intradermal, transdermal, subcutaneous, or intramuscular injections or intralesional, intracranial, intrapulmonal, intracardial, intraarticular and sublingual injections. More preferably, the vaccine may be administered by an intradermal, subcutaneous, or intramuscular route, preferably by injection, which may be needle-free and/or needle injection. Preferred in the context of the invention is intramuscular injection. Compositions/vaccines are therefore preferably formulated and stored in liquid form. The suitable amount of the vaccine to be administered can be determined by routine experiments, e.g. by using animal models. Such models include, without implying any limitation, rabbit, sheep, mouse, rat, dog and non-human primate models. Preferred unit dose forms for injection include sterile solutions of water, physiological saline or mixtures thereof. The pH should be adjusted to about 7.4.

The vaccine for multidose administration may be used according to the invention for human medical purposes and also for veterinary medical purposes (mammals, vertebrates, or avian species).

In embodiments, the vaccine for multidose administration elicits an adaptive immune response against at least one pathogen when administered to a cell or a subject, wherein the at least one pathogen may be selected from a bacterium, a protozoan, or a virus, for example from a pathogen provided in List 1.

In preferred embodiments the vaccine for multidose administration elicits an adaptive immune response against a Coronavirus (e.g. SARS-CoV-2) or a Rabies virus when administered to a cell or a subject.

In embodiments, administration of a therapeutically effective amount of the vaccine for multidose administration elicits neutralizing antibody titers against at least one pathogen, wherein the at least one pathogen may be selected from a bacterium, a protozoan, or a virus, for example from a pathogen provided in List 1. In preferred embodiments the vaccine for multidose administration elicits neutralizing antibody titers against a Coronavirus (e.g. SARS-CoV-2) or a Rabies virus when administered to a cell or a subject.

In embodiments, the neutralizing antibody titer that is induced upon administration of the vaccine for multidose administration to a subject is at least 100 neutralizing units per milliliter (NU/mL), at least 500 NU/mL, or at least 1000 NU/mL. In some embodiments, a neutralizing antibody titer of at least 100 NU/ml, at least 500 NU/ml, or at least 1000 NU/ml is produced in the serum of the subject at about 1 to about 72 hours post administration of the vaccine for multidose administration.

In some embodiments, the neutralizing antibody titer is sufficient to reduce infection with a pathogen by at least 50% relative to a neutralizing antibody titer of an unvaccinated control subject or relative to a neutralizing antibody titer of a subject vaccinated with a live attenuated viral vaccine, an inactivated viral vaccine, or a protein sub unit viral vaccine.

In some embodiments, the neutralizing antibody titer and/or the T cell immune response is sufficient to reduce the rate of asymptomatic pathogen (e.g. a Coronavirus or a Rabies virus) infection relative to the neutralizing antibody titer of unvaccinated control subjects.

In some embodiments, the neutralizing antibody titer and/or a T cell immune response is sufficient to prevent viral latency in the subject.

In preferred embodiments, administration of a therapeutically effective amount of the vaccine for multidose administration to a subject induces a T cell immune response against a pathogen (e.g. a Coronavirus or a Rabies virus) in the subject, preferably wherein the T cell immune response comprises a CD4+ T cell immune response and/or a CD8+ T cell immune response.

In some embodiments, the neutralizing antibody titer is sufficient to block fusion of a pathogen (e.g. a Coronavirus or a Rabies virus) with epithelial cells of the subject.

In some embodiments, the neutralizing antibody titer is induced within 20 days following a single 1 ug-100 ug dose of the vaccine for multidose administration, or within 40 days following a second 1 ug-100 μg dose of the vaccine for multidose administration, wherein, preferably, the dose relates to the amount of the RNA. In preferred embodiments, a dose comprises less that about 100 μg, preferably less than about 50 μg, more preferably less than about 20 μg, even more preferably less than about 10 μg, wherein preferably, the dose relates to the amount of the RNA.

In some embodiments, the vaccine comprises 5 to 100 does, preferably 10 to 50 doses.

In preferred embodiments, the vaccine for multidose administration elicits antigen-specific immune responses in a subject that has an age of about 5 years old or younger. Accordingly, the vaccine for multidose administration is particularly suitable for infants.

In preferred embodiments, the vaccine for multidose administration elicits antigen-specific immune responses in a subject that has an age of about 60 years old or older. Accordingly, the vaccine for multidose administration of the second aspect are particularly suitable for the elderly.

In preferred embodiments, the vaccine for multidose administration is stable or microbially preserved after a first dose withdrawal as defined in the context of the first aspect.

In preferred embodiments, the vaccine for multidose administration is stable or microbially preserved after addition of the least one antimicrobial preservative as defined in the context of the first aspect.

### Kit or Kit of Parts:

In a third aspect, the present invention provides a kit or kit of parts for preparing and/or administering a multidose composition or multidose vaccine.

In preferred embodiments, the kit or kit of parts is for preparing and/or administering 5 to 100 does, preferably 10 to 50 doses or 5 to 50 doses. In preferred embodiments, the kit or kit of parts is for preparing and/or administering 5 to 25 does, preferably 5 to 10 doses. Accordingly, kit or kit of parts is for preparing and/or administering 2, 3, 4, 5, 6, 7, 8, 9, 10, 11, 12, 13, 14, 15, 16, 17, 18, 19, 20, or more than 20 doses.

It has to be noted that features and embodiments that are described in the context of the pharmaceutical composition for multidose administration of the first aspect or the vaccine for multidose administration of the second aspect may also be applicable to the kit or kit of parts of the third aspect. Likewise, features and embodiments that are described in the context of the kit or kit of parts of the third aspect may also be applicable to the pharmaceutical composition for multidose administration of the first aspect or the vaccine for multidose administration of the second aspect.

In embodiments, the kit or kit of parts of the third aspect is configured to allow the preparation of the pharmaceutical composition for multidose administration of the first aspect or the vaccine for multidose administration of the second aspect.

In embodiments, the kit or kit of parts of the third aspect is configured to allow the administration of the pharmaceutical composition for multidose administration as defined in the first aspect or the vaccine for multidose administration defined in the second aspect.

In embodiments, the kit or kit of parts comprises technical instructions providing information on administration of the components and dosage of the components. The technical instructions of said kit may contain information about administration and dosage and patient groups. Further, the technical instructions may contain information about preparation of a multidose composition or vaccine.

Such kits, preferably kits of parts, may be applied e.g. for any of the applications or uses mentioned herein, preferably the treatment or prophylaxis of an infection or diseases caused by a (pandemic) pathogen, e.g. a Coronavirus, preferably SARS-CoV-2 coronavirus.

In preferred embodiments, the kit or kit of parts for preparing and/or administering a multidose composition or vaccine comprises the following components
Component A: at least one pharmaceutical composition comprising lipid-based carriers encapsulating an RNA; and
Component B: at least one sterile buffer for diluting component A, wherein the sterile dilution buffer comprises at least one antimicrobial preservative, or a combination thereof.

In preferred embodiments, component A and component B are provided in separate containers or vials.

In preferred embodiments, component A and component B are combined to dilute component A to obtain a multidose composition as defined in the first aspect or a multidose vaccine as defined in the second aspect.

In preferred embodiments, the dilution factor (component A:component B) is in a range from 1:1 to 1:50, preferably between 1:5 and 1:15. In specific embodiments, the dilution factor is 1:5, 1:6, 1:7, 1:8, 1:9, 1:10, 1:11, 1:12, 1:13, 1:14, 1:15, 1:16, 1:17, 1:18, 1:19, or 1:20. Suitably, the dilution factor is chosen to obtain a desired target concentration.

As an example, 1 ml of component A may be combined with 10 ml of component B with a dilution factor (component A: component B) of 1:11 to obtain 11 ml diluted multidose composition or a multidose vaccine.

In preferred embodiments, component B comprises at least one antimicrobial preservative, preferably least one antimicrobial preservative as defined in the context of the first aspect. Suitable embodiments antimicrobial preservative of component B are provided in the following. Further details regarding said embodiments, and further alternative embodiments, are defined in the context of the first aspect Suitably, the at least one antimicrobial preservative comprised in component B may be selected from bronopol, cetrimide, cetylpyridinium chloride, chlorhexidine, chlorobutanol, ethyl alcohol, hexetidine, imidurea, phenylmercuric nitrate, propylene glycol, an aromatic alcohol, a sugar alcohol, and/or thimerosal.

In preferred embodiments, component B comprises at least one antimicrobial preservative selected from at least one aromatic alcohol, at least one sugar alcohol, thiomersal, or a combination thereof.

In preferred embodiments, the at least one antimicrobial preservative of component B is selected from at least one aromatic alcohol.

Accordingly, in particularly preferred embodiments, the kit or kit of parts for preparing and/or administering a multidose composition or vaccine comprises the following components
Component A: at least one pharmaceutical composition comprising lipid-based carriers encapsulating an RNA; and
Component B: at least one sterile buffer for diluting component A, wherein the sterile dilution buffer comprises at least one antimicrobial preservative selected from at least one aromatic alcohol.

In preferred embodiments, the at least one aromatic alcohol of component B is selected from benzyl alcohol, phenoxyethanol, phenylethyl alcohol, or a combination thereof.

In preferred embodiments, the least one antimicrobial preservative of component B is selected from at least one aromatic alcohol, wherein the at least one aromatic alcohol is phenoxyethanol.

In embodiments, the at least one aromatic alcohol (e.g. phenoxyethanol) contained in component B is in a concentration of about 0.1% (w/v) to about 5% (w/v), preferably in a concentration of about 0.1% (w/v) to about 2%.

In preferred embodiments, the at least one aromatic alcohol (e.g. phenoxyethanol) is in a concentration of about 0.5% (w/v) to about 1% (w/v).

As component B is used to dilute component A to obtain a composition or vaccine for multidose administration as defined herein, the concentration of the at least one aromatic alcohol of component B depends on the dilution factor.

In specific embodiments, the at least one aromatic alcohol (e.g. phenoxyethanol) contained in component B is in a concentration of 0.1% (w/v), 0.2% (w/v), 0.3% (w/v), 0.4% (w/v), 0.5% (w/v), 0.6% (w/v), 0.7% (w/v), 0.8% (w/v), 0.9% (w/v), 1% (w/v), 1.1% (w/v), 1.2% (w/v), 1.3% (w/v), 1.4% (w/v), 1.5% (w/v), 1.6% (w/v), 1.7% (w/v), 1.8% (w/v), 1.9% (w/v), or 2% (w/v).

In preferred embodiments, the at least one antimicrobial preservative of component B selected from thiomersal.

In embodiments, thiomersal is in a concentration of about 0.0005% (w/v) to about 0.1% (w/v), preferably in a concentration of about 0.0005% (w/v) to about 0.05% (w/v).

As component B is used to dilute component A to obtain a composition or vaccine for multidose administration as defined herein, the concentration of thiomersal of component B depends on the dilution factor.

In embodiments, thiomersal contained in component B is in a concentration of 0.0005% (w/v), 0.001% (w/v), 0.0015% (w/v), 0.002% (w/v), 0.0025% (w/v), 0.003% (w/v), 0.0035% (w/v), 0.004% (w/v), 0.0045% (w/v), 0.005% (w/v), 0.0055% (w/v), 0.006% (w/v), 0.0065% (w/v), 0.007% (w/v), 0.0075% (w/v), 0.008% (w/v), 0.0085% (w/v), 0.009% (w/v), 0.0095% (w/v), 0.01% (w/v), 0.011% (w/v), 0.012% (w/v), 0.013% (w/v), 0.014% (w/v), 0.015% (w/v), 0.016% (w/v), 0.017% (w/v), 0.018% (w/v), 0.019% (w/v), 0.020% (w/v).

In preferred embodiments, the at least one antimicrobial preservative of component B is selected from at least one sugar alcohol.

In preferred embodiments, the at least one sugar alcohol of component B is selected from xylitol, sorbitol, and/or glycerol, or a combination thereof.

In particularly preferred embodiments, the at least one sugar alcohol of component B is xylitol.

In embodiments, the at least one sugar alcohol (e.g. xylitol) is in a concentration of about 10 mM to about 500 mM, preferably in a concentration of about 10 mM to about 200 mM, more preferably in a concentration of about 25 mM to about 200 mM, even more preferably in a concentration of about 25 mM to about 150 mM As component B is used to dilute component A to obtain a composition or vaccine for multidose administration as defined herein, the concentration the at least one sugar alcohol (e.g. xylitol) of component B depends on the dilution factor.

In specific embodiments, the at least one sugar alcohol (e.g. xylitol) contained in component B is in a concentration of 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, 100 mM, 110 mM, 120 mM, 130 mM, 140 mM, 150 mM, 160 mM, 170 mM, 180 mM, 190 mM, or 200 mM.

In preferred embodiments, component B comprises phenoxyethanol and, optionally, at least one sugar alcohol, preferably xylitol.

In embodiments, component B comprises more than one, preferably 2, 3, 4, 5, 6 or more of the antimicrobial preservatives as defined above.

In particularly preferred embodiments, component B comprises at least two antimicrobial preservatives selected from at least one aromatic alcohol and from at least one sugar alcohol.

In preferred embodiments, the at least one aromatic alcohol of component B is selected from benzyl alcohol, phenoxyethanol, phenylethyl alcohol, or a combination thereof, and the at least one sugar alcohol of component B is selected from xylitol, sorbitol, and/or glycerol, or a combination thereof.

In preferred embodiments, component B comprises phenoxyethanol and xylitol.

In embodiments where at least one aromatic alcohol (e.g. phenoxyethanol) and at least one sugar alcohol (e.g. xylitol) are contained in component B, the at least one aromatic alcohol (e.g. phenoxyethanol) is in a concentration of about 0.1% (w/v) to about 5% (w/v), preferably in a concentration of about 0.1% (w/v) to about 2%, and the at least one sugar alcohol (e.g. xylitol) is in a concentration of about 10 mM to about 500 mM, preferably in a concentration of about 10 mM to about 200 mM, more preferably in a concentration of about 25 mM to about 200 mM, even more preferably in a concentration of about 25 mM to about 150 mM In preferred embodiments, component B of the kit or kit of parts comprises phenoxyethanol and xylitol, wherein phenoxyethanol is in a concentration of about 0.1% (w/v) to about 2% (w/v), preferably 0.5% (w/v), and wherein xylitol is in a concentration of about 10 mM to about 200 mM, preferably about 25 mM to about 150 mM.

In preferred embodiments, component B comprises a salt, preferably NaCL. In preferred embodiments, component B comprises a salt, preferably NaCL in a concentration of about 0.9%.

As outlined above, component B comprises at least one sterile dilution buffer for diluting component A. Suitably, component B is a heat autoclaved sterile dilution buffer. Accordingly, it is preferred that the antimicrobial preservative used in component B can be heat autoclaved (e.g., that the antimicrobial preservative does not lose its antimicrobial function).

In preferred embodiments, component B is provided as a liquid.

In preferred embodiments, component A comprises at least one pharmaceutical composition comprising lipid-based carriers encapsulating an RNA, wherein the RNA is preferably as defined in the context of the first aspect. Suitable embodiments regarding the RNA of component A are provided in the following. Further details regarding said embodiments, and further alternative embodiments, are defined in the context of the first aspect.

In embodiments, the concentration of the RNA comprised in component A is in a range from about 10 μg/ml to about 10 mg/ml, preferably in a range from about 100 μg/ml to about 1 mg/ml.

In preferred embodiments, the RNA comprised in component A has an RNA integrity of at least about 50%, preferably of at least about 60%, more preferably of at least about 70%, most preferably of at least about 80%. RNA is suitably determined analytical HPLC, preferably analytical RP-HPLC.

In embodiments, component A comprises about 80% encapsulated RNA (and about 20% free RNA), about 85% encapsulated RNA (and about 15% free RNA), about 90% encapsulated RNA (and about 10% free RNA), or about 95% encapsulated RNA (and 5% about free RNA).

In various embodiments, component A comprises purified RNA. It may be suitably to apply certain purification steps during RNA production to achieve certain RNA purity levels in regards of various impurities. Accordingly, the RNA used for formulation of the lipid-based carriers has been purified (before formulation/encapsulation) to remove various RNA impurities.

In embodiments, the RNA of component A is an RP-HPLC purified RNA and/or a tangential flow filtration (TFF) purified RNA.

In embodiments, the RNA of component A comprises (chemically) modified nucleotides.

In preferred embodiments, the RNA of component A does not comprise (chemically) modified nucleotides as defined herein.

In various embodiments, the RNA of component A ion is a therapeutic RNA. Accordingly, the RNA encapsulated in lipid based carriers may be a therapeutic RNA.

In preferred embodiments, the RNA of component A is a coding RNA. Most preferably, said coding RNA may be selected from an mRNA, a (coding) self-replicating RNA, a (coding) circular RNA, a (coding) viral RNA, or a (coding) replicon RNA.

In particularly preferred embodiments, the RNA of component A is an mRNA.

Accordingly, in preferred embodiments, the RNA of component A comprises at least one coding sequence.

In preferred embodiments, the RNA of component A comprises at least one codon modified coding sequence.

In preferred embodiments, the at least one coding sequence of the RNA of component A is a codon modified coding sequence, wherein the codon modified coding sequence is selected from C maximized coding sequence, CAI maximized coding sequence, human codon usage adapted coding sequence, G/C content modified coding sequence, and G/C optimized coding sequence, or any combination thereof.

In embodiments, the RNA of component A has a GC content of about 50% to about 80%.

In various embodiments, the coding sequence of the RNA of component A has a GC content of about 60% to about 90%.

In preferred embodiments, the RNA of component A composition comprises a 5′-cap structure, preferably a cap1 structure.

In preferred embodiments, the RNA of component A comprises at least one poly(A) sequence, and/or at least one poly(C) sequence, and/or at least one histone stem-loop and/or at least one 5′-UTR and/or at least one 3′-UTR.

In preferred embodiments, the RNA of component A comprises at least one heterologous untranslated region (UTR).

In embodiments, the RNA of component A comprises at least one 5′-UTR, preferably a heterologous 5′-UTR and/or at least one 3′-UTR, preferably a heterologous 3′-UTR.

In particularly preferred embodiments, the RNA of component A comprises at least one coding sequence as defined wherein said coding sequence is operably linked to a HSD17B4 5′-UTR and a PSMB3 3′-UTR (HSD17B4/PSMB3).

In particularly preferred embodiments, the RNA of component A comprises at least one coding sequence as defined herein, wherein said coding sequence is operably linked to an alpha-globin (“muag”) 3′-UTR.

In various embodiments, the RNA of component A comprises at least one poly(N) sequence, e.g. at least one poly(A) sequence, at least one poly(U) sequence, at least one poly(C) sequence, or combinations thereof.

In embodiments, the RNA of component A comprises at least one histone stem-loop (hSL) or histone stem loop structure.

In various embodiments the RNA of component A comprises, preferably in 5′- to 3′-direction, the following elements:
A) 5′-cap structure, preferably as specified herein;
B) 5′-terminal start element, preferably as specified herein;
C) optionally, a 5′-UTR, preferably as specified herein;
D) a ribosome binding site, preferably as specified herein;
E) at least one coding sequence, preferably as specified herein;
F) 3′-UTR, preferably as specified herein;
G) optionally, poly(A) sequence, preferably as specified herein;
H) optionally, poly(C) sequence, preferably as specified herein;
I) optionally, histone stem-loop preferably as specified herein;
J) optionally, 3′-terminal sequence element, preferably as specified herein.

In preferred embodiments, the RNA of component A comprises at least one coding sequence encoding at least one peptide or protein suitable for use in treatment or prevention of a disease, disorder or condition.

In embodiments, the peptide or protein encoded by the RNA of component A is selected from an antigen or epitope of a pathogen selected or derived from List 1 of the first aspect.

In embodiments, the peptide or protein encoded by the RNA of component A is selected from an antigen or epitope of a pathogen selected or derived from a (pandemic) Coronavirus, e.g. SARS-CoV-2, or a fragment or variant of any of these.

According to preferred embodiments, the RNA of component A comprises a coding sequence encoding at least one antigen or epitope selected or derived from a (pandemic) Coronavirus, preferably SARS-CoV-2.

In embodiments, the peptide or protein encoded by the RNA of component A is selected from an antigen or epitope of a pathogen selected or derived from a Rabies virus, or a fragment or variant of any of these.

According to preferred embodiments, the RNA of component A comprises a coding sequence encoding at least one antigen or epitope selected or derived from a Rabies virus.

In preferred embodiments, component A comprises at least one pharmaceutical composition comprising lipid-based carriers encapsulating an RNA, wherein the lipid-based carriers are preferably as defined in the context of the first aspect.

Suitable embodiments regarding the lipid-based carriers of component A are provided in the following. Further details regarding said embodiments, and further alternative embodiments, are defined in the context of the first aspect.

In a preferred embodiment, the concentration of lipid (or lipid-based carriers) in component A is in a range from about 250 μg/ml to about 250 mg/ml.

In embodiments, the weight to weight (wt/wt) ratio of lipid to the RNA (in the lipid-based carriers) of component A is from about 10:1 to about 60:1. In particularly preferred embodiments, the wt/wt ratio of lipid to the RNA (in the lipid-based carriers) is about 25:1.

In embodiments, the RNA to total lipid ratio in the lipid based carriers of component A is between about 0.03 w/w and 0.05 w/w.

In embodiments, the N/P ratio of the lipid-based carriers to the RNA of component A is in a range from about 1 to about 10, preferably in a range from about 1 to about 7, more preferably in a range from about 5 to about 7, e.g. about 5.5, about 5.6, about 5.7, about 5.8, about 5.9, about 6, about 6.1, about 6.2, bout 6.3, about 6.4, about 6.5. In preferred embodiments, the N/P ratio of the lipid-based carriers to the RNA is about 6.

In various embodiments, lipid-based carriers encapsulating the RNA of component A are monodisperse, meaning that the lipid-based carriers comprised in the composition have a uniform size. Typically, the distribution of size populations within a composition is expressed by the polydispersity index (PDI) value.

In embodiments, the lipid-based carriers encapsulating the RNA of component A have as a polydispersity index (PDI) value ranging from about 0.50 to about 0.00. In embodiments, the lipid-based carriers encapsulating the RNA have a polydispersity index (PDI) value of less than about 0.3, preferably of less than about 0.2, more preferably of less than about 0.15, most preferably of less than about 0.1.

In preferred embodiments, the lipid-based carriers encapsulating the RNA of component A have a Z-average size ranging from about 50 nm to about 150 nm, preferably in a range from about 50 nm to about 120 nm, more preferably in a range from about 60 nm to about 115 nm. Suitably, the Z-average size may be determined by DLS as commonly known in the art.

In embodiments, the lipid-based carriers encapsulating the RNA of component A are a liposomes, lipid nanoparticles, lipoplexes, and/or nanoliposomes. In preferred embodiments, the lipid-based carriers encapsulating the RNA of component A are a lipid nanoparticle (LNP).

In preferred embodiments, the lipid-based carriers (e.g. LNPs) encapsulating the RNA of component A comprise at least two lipid components, at least three lipid components, preferably at least four lipid components, wherein the lipid components may be selected from at least one aggregation-reducing lipid, at least one cationic lipid, at least one neutral lipid, and/or at least one steroid or steroid analog.

In preferred embodiments, the lipid-based carriers (e.g. LNPs) encapsulating the RNA of component A comprise at least one aggregation-reducing lipid, at least one cationic lipid, at least one neutral lipid, and/or at least one steroid or steroid analog.

In embodiments, the lipid-based carriers encapsulating the RNA of component A comprise at least one aggregation reducing lipid as defined in the first aspect.

In preferred embodiments, the lipid-based carriers (e.g. the LNPs) encapsulating the RNA of component A comprise a PEG-conjugated lipid, wherein said PEG-conjugated lipid is a lipid according to formula (IVa) or derived from formula (IVa):

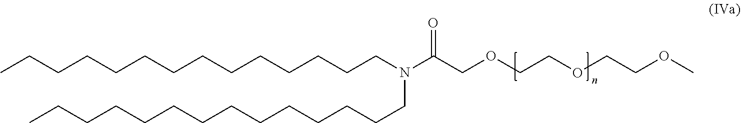

wherein n has a mean value ranging from 30 to 60, such as about 30±2, 32±2, 34±2, 36±2, 38±2, 40±2, 42±2, 44±2, 46±2, 48±2, 50±2, 52±2 54±2, 56±2, 58±2, or 60±2. In a preferred embodiment n is about 49. In another preferred embodiment n is about 45.

Further examples of PEG-conjugated lipids suitable in that context are provided in US2015/0376115A1 and WO2015/199952, each of which is incorporated by reference in its entirety.

In preferred embodiments, the lipid-based carriers encapsulating the RNA of component A comprise at least one cationic lipid as defined in the first aspect.

In particularly preferred embodiments, the lipid-based carriers (e.g. LNPs) encapsulating the RNA of component A comprise a cationic lipid according to formula (III-3) or derived from formula (III-3):

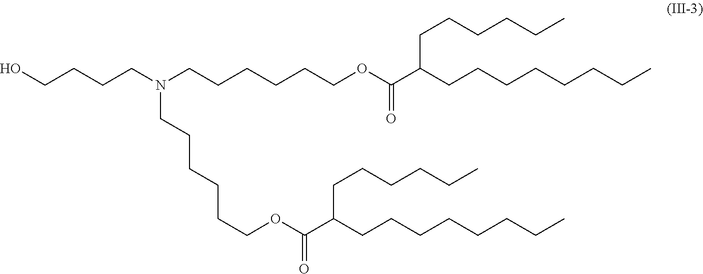 [SMILES: CCCCCCCCC(CCCCCC)C(=O)OCCCCCCN(CCCCO)CCCCCCOC(=O)C(CCCCCC)CCCCCCCC]

In certain embodiments, the cationic lipid of component A as defined herein, more preferably cationic lipid compound III-3, is present in the lipid-based carriers encapsulating the RNA in an amount from about 30 to about 95 mole percent, relative to the total lipid content of the lipid-based carrier. If more than one cationic lipid is incorporated within the lipid-based carrier, such percentages apply to the combined cationic lipids.

In preferred embodiments, the lipid-based carriers encapsulating the RNA of component A comprise at least one neutral lipid as defined in the first aspect.

In preferred embodiments, the neutral lipid of the lipid-based carriers encapsulating the RNA of component A is 1,2-distearoyl-sn-glycero-3-phosphocholine (DSPC).

In preferred embodiments, the lipid-based carriers encapsulating the RNA of component A comprise at least one steroid or steroid analog as defined in the first aspect.

In embodiments, the steroid or steroid analog of the lipid-based carriers encapsulating the RNA of component A is cholesterol.

In particularly preferred embodiments, the lipid-based carriers encapsulating the RNA of component A comprises
(v) at least one cationic lipid as defined herein, preferably a lipid of formula (III), more preferably lipid III-3;
(vi) at least one neutral lipid as defined herein, preferably 1,2-distearoyl-sn-glycero-3-phosphocholine (DSPC);
(vii) at least one steroid or steroid analog as defined herein, preferably cholesterol; and
(viii) at least one aggregation reducing lipid, preferably a polymer-conjugated lipid, more preferably a PEG-conjugated lipid derived from formula (IVa).

In preferred embodiments, the lipid-based carriers encapsulating the RNA of component A comprise (i) to (iv) in a molar ratio of about 20-60% cationic lipid, about 5-25% neutral lipid, about 25-55% steroid or steroid analogue, and about 0.5-15% aggregation reducing lipid, e.g. polymer conjugated lipid.

In embodiments, component A comprises the lipid-based carriers encapsulating the RNA which have a molar ratio of approximately 50:10:38.5:1.5, preferably 47.5:10:40.8:1.7 or more preferably 47.4:10:40.9:1.7 (i.e. proportion (mol %) of cationic lipid (preferably lipid III-3), DSPC, cholesterol, and aggregation reducing lipid (e.g. polymer conjugated lipid, preferably PEG-lipid (preferably PEG-lipid of formula (IVa) with n=49 or with n=45))).

In embodiments, component A comprising the lipid-based carriers encapsulating the RNA has a turbidity ranging from about 150 FNU to about 0.0 FNU. In embodiments, the composition has a turbidity of about 100 FNU or less, preferably of about 50 FNU or less, more preferably of about 25 FNU or less.

In embodiments, component A comprises a buffer e.g. comprising a sugar and/or a salt and/or a buffering agent.

In embodiments, component A comprises a sugar, preferably a disaccharide. In embodiments, the concentration of the sugar comprised in the composition is in a range from about 5 mM to about 300 mM. In embodiments, the sugar comprised in the composition is sucrose, preferably in a concentration of about 150 mM.

In embodiments, component A comprises a salt, preferably NaCl. In embodiments, the concentration of the salt comprised in the composition is in a range from about 10 mM to about 300 mM, preferably about 150 mM. In embodiments, the salt comprised in composition is NaCl, preferably in a concentration of about 75 mM.

In embodiments, component A comprises a buffering agent, preferably selected from Tris, HEPES, NaPO4 or combinations thereof. In embodiments, the buffering agent is in a concentration ranging from about 0.1 mM to about 100 mM. In embodiments, the buffering agent is NaPO4, preferably in a concentration of about 1 mM. In other embodiments, the buffering agent is Tris.

In embodiments, component A has a pH in a range of about pH 7.0 to about pH 8.0. In preferred embodiments, the composition has a pH of about pH 7.4.

In embodiments, component A has an osmolality of about 250 mOsmol/kg to about 450 mOsmol/kg, preferably of about 335 mOsmol/kg. The Osmolarity of the composition may be determined by the skilled person using an osmometer.

In a preferred embodiment, component A comprises a sugar in a concentration of about 50 mM to about 300 mM, preferably sucrose in a concentration of about 150 mM.

In a preferred embodiment, component A comprises a salt in a concentration of about 10 mM to about 200 mM, preferably NaCl in a concentration of about 75 mM.

In embodiments, component A is provided as a liquid.

In embodiments, component A is provided as a lyophilized composition (e.g. wherein lyophilization is performed according to WO2016/165831 or WO2011/069586) or as a spray-freeze dried composition or as a spray dried composition (e.g. wherein spray-freeze drying or spray drying is performed according to WO2016/184575 or WO2016/184576). Accordingly, in that context the disclosures of WO2016/165831, WO2011/069586, WO2016/184575, and WO2016/184576 are incorporated herewith by reference.

In embodiments where component A is provided as a lyophilized or spray-freeze dried or spray dried composition, component B may be used for re-constitution to obtain a pharmaceutical composition or vaccine for multidose administration.

In preferred embodiments, the kit or kit of parts comprises at least one means for combining component A and component B. Preferably, the means for combining is a syringe.

In preferred embodiments, the kit or kit of parts comprises at least one means for withdrawal of a dose and/or for administering the prepared composition or vaccine, preferably the diluted and microbially preserved composition or vaccine, to a subject. Preferably, the means for withdrawal of a dose and/or for administering is a syringe.

Suitably, the syringe for withdrawal of a dose and/or for administering is configured for intramuscular administration to a subject, e.g. a human subject.

In preferred embodiments, the kit or kit of parts of the third aspect is configured for a multidose-administration.

In embodiments, the containers or vials used for administering of the composition or vaccine is configured to allow multiple withdrawals of an effective dose by a syringe, preferably by a syringe for administering to a subject (e.g. a syringe for intramuscular administration).

In preferred embodiments of the third aspect, the kit or kit of parts comprises the following components:
(A) at least one pharmaceutical composition comprising lipid-based carriers encapsulating an RNA as defined herein; and
(B) at least one sterile dilution buffer for diluting component A as defined herein, wherein the sterile dilution buffer comprises at least one antimicrobial preservative selected from at least one aromatic alcohol, at least one sugar alcohol, thiomersal, or a combination thereof, preferably at least one aromatic alcohol
(C) optionally, at least one means for combining component A and B to obtain a pharmaceutical composition or vaccine for multidose administration; and
(D) at least one syringe for administering the obtained pharmaceutical composition or vaccine for multidose administration to a subject, preferably configured for intramuscular administration to a human subject

In preferred embodiments, the obtained pharmaceutical composition or vaccine for multidose administration is stable for at least 6 hours, preferably for at least about 1 day after combining component A and component B. In preferred embodiments, the obtained pharmaceutical composition or vaccine for multidose administration is stable for at least 6 hours to about 6 months after combining component A and component B. In embodiments, the obtained pharmaceutical composition for multidose administration is stable for at least 1 day, 2 days, 3 days, 4 days, 5 days, 6 days, 7 days, 2 weeks, 3 weeks, 4 weeks, 1 month, 2 months, 3 months, 4 months, 5 months, or 6 months after combining component A and component B.

In preferred embodiments, the obtained pharmaceutical composition or vaccine for multidose administration is stable at a temperature of about 5° C. to about 25° C. after combining component A and component B. In embodiments, the obtained pharmaceutical composition or vaccine for multidose administration is stable at a temperature of about 5° C., 6° C., 7° C., 8° C., 9° C., 10° C., 15° C., 20° C., or 25° C. after combining component A and component B.

In preferred embodiments, after combining component A and component B, the integrity of the RNA decreases less than about 30%, preferably less than about 20%, more preferably less than about 10%. RNA is suitably determined using analytical HPLC, preferably analytical RP-HPLC. In preferred embodiments, after combining component A and component B, the RNA has an RNA integrity ranging from about 40% to about 100%. RNA integrity is suitably determined using analytical HPLC, preferably analytical RP-HPLC.

In preferred embodiments, after combining component A and component B, the amount of free RNA does not increase by more than 20%, preferably by not more than 10%, more preferably by not more than 5%. In embodiments, after combining component A and component B, the amount of free RNA in the composition ranges from about 30% to about 0%. Values are compared to the respective value determined for component A alone. Free RNA is suitably determined using a RiboGreen assay.

In embodiments, after combining component A and component B, the percentage of RNA encapsulation does not decrease by more than 20%, preferably by not more than 10%. In embodiments, after combining component A and component B, the percentage of RNA encapsulation ranges from about 60% to about 100%. Values are compared to the respective value determined for component A alone. RNA encapsulation is suitably determined using a RiboGreen assay.

In embodiments, after combining component A and component B, the PDI value does not increase by more than a value of about 0.2, preferably by not more than a value of about 0.1. In embodiments, after combining component A and component B, the PDI value ranges from about 0.4 to about 0.0. Values are compared to the respective value determined for component A alone. PDI is suitably determined using DLS In embodiments, after combining component A and component B, the Z-average size of the lipid based carriers encapsulating the RNA does not increase by more than 20%, preferably by not more than 10%. In embodiments, after combining component A and component B, the Z-average size of the lipid based carriers encapsulating the RNA ranges from about 50 nm to about 150 nm. Values are compared to the respective value determined for component A alone. Z-average size is suitably determined using DLS.

In embodiments, after combining component A and component B, the potency of the composition or vaccine decreases less than about 30%, preferably less than 20%, more preferably less than 10%. In embodiments, after combining component A and component B, the reactogenicity of the composition or vaccine does not increase by more than 20%, preferably by not more than 10%. Values are compared to the respective value determined for component A alone.

In preferred embodiments, after combining component A and component B, the obtained pharmaceutical composition or vaccine for multidose administration is microbially preserved for at least 6 hours, preferably for at least about 1 day. In preferred embodiments, the pharmaceutical composition for multidose administration is microbially preserved for at least 6 hours to about 6 months. In embodiments, the obtained pharmaceutical composition or vaccine for multidose administration is microbially preserved for at least 1 day, 2 days, 3 days, 4 days, 5 days, 6 days, 7 days, 2 weeks, 3 weeks, 4 weeks, 1 month, 2 months, 3 months, 4 months, 5 months, or 6 months.

In embodiments, after combining component A and component B, the obtained composition or vaccine is microbially preserved, preferably for at least about 1 day and/or at a temperature of about 5° C. to about 25° C. and/or for at least about 1 day after a first dose withdrawal.

In preferred embodiments, after combining component A and component B, the obtained pharmaceutical composition or vaccine for multidose administration is microbially preserved at a temperature of about 5° C. to about 25° C. In embodiments, the obtained pharmaceutical composition or vaccine for multidose administration is microbially preserved at a temperature of about 5° C., 6° C., 7° C., 8° C., 9° C., 10° C., 15° C., 20° C., or 25° C.

In preferred embodiments, the obtained pharmaceutical composition or vaccine for multidose administration as described in the context of the first aspect or second aspect is for parental use, preferably for multi-dose parenteral use.

### First, Second, and Further Medical Use:

In a fourth aspect, the present invention relates to the medical use of the pharmaceutical composition for multidose administration of the first aspect, the vaccine for multidose administration of the second aspect, or the kit or kit of parts of the third aspect.

Notably, embodiments relating to the composition of the first aspect, the vaccine of the second aspect, or the kit or kit of parts of the third aspect may likewise be read on and be understood as suitable embodiments of medical uses of the invention.

Accordingly, the invention provides a pharmaceutical composition for multidose administration as defined in the first aspect for use as a medicament, a vaccine for multidose administration as defined in the second aspect for use as a medicament, or a kit or kit of parts as defined in the third aspect for use as a medicament.

In embodiments, the pharmaceutical composition for multidose administration of the first aspect, the vaccine for multidose administration of the second aspect, or the kit or kit of parts of the third aspect may be used for human medical purposes and also for veterinary medical purposes, preferably for human medical purposes.

In embodiments, the pharmaceutical composition for multidose administration of the first aspect, the vaccine for multidose administration of the second aspect, or the kit or kit of parts of the third aspect may be in particular used and most suitable for human medical purposes, in particular for young infants, new-borns, immunocompromised recipients, pregnant and breast-feeding women, and elderly people.

In yet another aspect, the invention relates to the pharmaceutical composition for multidose administration of the first aspect, the vaccine for multidose administration of the second aspect, or the kit or kit of parts of the third aspect for use in the treatment or prophylaxis of a tumour disease, or of a disorder related to such tumour disease.

Accordingly, in said embodiments, the RNA may encode at least one tumour or cancer antigen and/or at least one therapeutic antibody (e.g. checkpoint inhibitor).

In yet another aspect, the invention relates to the pharmaceutical composition for multidose administration of the first aspect, the vaccine for multidose administration of the second aspect, or the kit or kit of parts of the third aspect for use in the treatment or prophylaxis of a genetic disorder or condition.

Such a genetic disorder or condition may be a monogenetic disease, i.e. (hereditary) disease, or a genetic disease in general, diseases which have a genetic inherited background and which are typically caused by a defined gene defect and are inherited according to Mendel's laws.

Accordingly, in said embodiments, the RNA may encode a CRISPR-associated endonuclease or another protein or enzyme suitable for genetic engineering.

In yet another aspect, the invention relates to the pharmaceutical composition for multidose administration of the first aspect, the vaccine for multidose administration of the second aspect, or the kit or kit of parts of the third aspect.

Accordingly, in said embodiments, the RNA may encode at least one protein or enzyme. “Protein or enzyme deficiency” in that context has to be understood as a disease or deficiency where at least one protein is deficient, e.g. A1AT deficiency.

In yet another aspect, the invention relates to the pharmaceutical composition for multidose administration of the first aspect, the vaccine for multidose administration of the second aspect, or the kit or kit of parts of the third aspect for use in the treatment or prophylaxis of autoimmune diseases, allergies or allergic diseases, cardiovascular diseases, neuronal diseases, diseases of the respiratory system, diseases of the digestive system, diseases of the skin, musculoskeletal disorders, disorders of the connective tissue, neoplasms, immune deficiencies, endocrine, nutritional and metabolic diseases, eye diseases, and ear diseases.

In yet another aspect, the invention relates to the pharmaceutical composition for multidose administration of the first aspect, the vaccine for multidose administration of the second aspect, or the kit or kit of parts of the third aspect for use in the treatment or prophylaxis of an infection, or of a disorder related to such an infection.

In that context, an infection may be caused a pathogen selected from a bacterium, a protozoan, or a virus, for example from a pathogen provided in List 1. In preferred embodiments, the pathogen is a virus, e.g. a Coronavirus (e.g. SARS-CoV-2) or a Rabies virus. In preferred embodiments, the pathogen is a pandemic pathogen, e.g. a pandemic virus.

Accordingly, the invention relates to the pharmaceutical composition for multidose administration of the first aspect, the vaccine for multidose administration of the second aspect, or the kit or kit of parts of the third aspect for use in the treatment or prophylaxis of an infection with a Coronavirus, preferably a SARS-CoV-2 coronavirus, or of a disorder related to such an infection.

In the context of the medical uses, the pharmaceutical composition for multidose administration of the first aspect, the vaccine for multidose administration of the second aspect, or the kit or kit of parts of the third aspect may preferably be administered locally or systemically. In that context, administration may be by an intradermal, subcutaneous, intranasal, or intramuscular route. In embodiments, administration may be by conventional needle injection or needle-free jet injection. Preferred is intramuscular injection. Alternatively, administration may be intravenous.

In embodiments, the RNA as comprised in the pharmaceutical composition for multidose administration of the first aspect, the vaccine for multidose administration of the second aspect, or the kit or kit of parts of the third aspect is provided in an amount of about 100 ng to about 500 ug, in an amount of about 1 ug to about 200 ug, in an amount of about 1 ug to about 100 ug, in an amount of about 5 ug to about 100 ug, preferably in an amount of about 10 ug to about 50 ug, specifically, in an amount of about 1 ug, 2 ug, 3 ug, 4 ug, 5 ug, 10 ug, 15 ug, 20 ug, 25 ug, 30 ug, 35 ug, 40 ug, 45 ug, 50 ug, 55 ug, 60 ug, 65 ug, 70 ug, 75 ug, 80 ug, 85 ug, 90 ug, 95 ug or 100 ug. Notably, the amount relates to the total amount of RNA of one dose comprised in the composition or vaccine.

In the context of a use in the treatment or prophylaxis of an infection, the immunization protocol for the treatment or prophylaxis of a subject against at least one pathogen, e.g. against a Coronavirus, preferably SARS-CoV-2 comprises one single dose. In some embodiments, the effective amount is a dose of 1 ug administered to the subject in one vaccination. In some embodiments, the effective amount is a dose of 2 ug administered to the subject in one vaccination. In some embodiments, the effective amount is a dose of 3 ug administered to the subject in one vaccination. In some embodiments, the effective amount is a dose of 4 ug administered to the subject in one vaccination. In some embodiments, the effective amount is a dose of 5 ug administered to the subject in one vaccination. In some embodiments, the effective amount is a dose of 10 ug administered to the subject in one vaccination. In some embodiments, the effective amount is a dose of 12 ug administered to the subject in one vaccination. In some embodiments, the effective amount is a dose of 20 ug administered to the subject in one vaccination. In some embodiments, the effective amount is a dose of 30 ug administered to the subject in one vaccination. In some embodiments, the effective amount is a dose of 40 ug administered to the subject in one vaccination. In some embodiments, the effective amount is a dose of 50 ug administered to the subject in one vaccination. In some embodiments, the effective amount is a dose of 100 ug administered to the subject in one vaccination. In some embodiments, the effective amount is a dose of 200 ug administered to the subject in one vaccination. Notably, the effective amount relates to the total amount of nucleic acid comprised in the composition or vaccine.

In the context of a use in the treatment or prophylaxis of an infection, the effective amount is a dose of 1 ug administered to the subject a total of two times. In some embodiments, the effective amount is a dose of 2 ug administered to the subject a total of two times. In some embodiments, the effective amount is a dose of 3 ug administered to the subject a total of two times. In some embodiments, the effective amount is a dose of 4 ug administered to the subject a total of two times. In some embodiments, the effective amount is a dose of 5 ug administered to the subject a total of two times. In some embodiments, the effective amount is a dose of 10 ug administered to the subject a total of two times. In some embodiments, the effective amount is a dose of 12 ug administered to the subject a total of two times. In some embodiments, the effective amount is a dose of 20 ug administered to the subject a total of two times. In some embodiments, the effective amount is a dose of 30 ug administered to the subject a total of two times. In some embodiments, the effective amount is a dose of 40 ug administered to the subject a total of two times. In some embodiments, the effective amount is a dose of 50 ug administered to the subject a total of two times. In some embodiments, the effective amount is a dose of 100 ug administered to the subject a total of two times. In some embodiments, the effective amount is a dose of 200 ug administered to the subject a total of two times. Notably, the effective amount relates to the total amount of RNA comprised in the composition or vaccine.

In the context of the invention, composition for multidose administration of the first aspect, the vaccine for multidose administration of the second aspect, or the kit or kit of parts of the third aspect may provide up to 5 effective doses, up to 10 effective doses, up to 20 effective doses, up to 40 effective doses, or up to 50 effective doses.

In preferred embodiments, the vaccination/immunization immunizes the subject against a pathogen infection, e.g. against a Coronavirus infection (upon administration as defined herein) for at least 1 year, preferably at least 2 years. In preferred embodiments, the vaccine/composition immunizes the subject against a pathogen infection, e.g. against a Coronavirus infection (upon administration as defined herein) for more than 2 years, more preferably for more than 3 years, even more preferably for more than 4 years, even more preferably for more than 5-10 years.

### Method of Treating or Preventing a Disorder:

In another aspect, the present invention relates to a method of treating or preventing a disorder or condition.

Notably, embodiments relating to the pharmaceutical composition for multidose administration of the first aspect, the vaccine for multidose administration of the second aspect, or the kit or kit of parts of the third aspect may likewise be read on and be understood as suitable embodiments of methods of treatment and use as provided herein. Furthermore, specific features and embodiments relating to method of treatments as provided herein may also apply for medical uses of the invention.

Preventing (Inhibiting) or treating a disease, in particular a virus infection relates to inhibiting the full development of a disease or condition, for example, in a subject who is at risk for a disease such as a virus infection. “Treatment” refers to a therapeutic intervention that ameliorates a sign or symptom of a disease or pathological condition after it has begun to develop. The term “ameliorating”, with reference to a disease or pathological condition, refers to any observable beneficial effect of the treatment. Inhibiting a disease can include preventing or reducing the risk of the disease, such as preventing or reducing the risk of viral infection. The beneficial effect can be evidenced, for example, by a delayed onset of clinical symptoms of the disease in a susceptible subject, a reduction in severity of some or all clinical symptoms of the disease, a slower progression of the disease, a reduction in the viral load, an improvement in the overall health or well-being of the subject, or by other parameters that are specific to the particular disease. A “prophylactic” treatment is a treatment administered to a subject who does not exhibit signs of a disease or exhibits only early signs for the purpose of decreasing the risk of developing pathology.

In preferred embodiments, the disorder is an infection with a pathogen, e.g. a pandemic pathogen, selected from a bacterium, a protozoan, or a virus, for example from a pathogen provided in List 1. In preferred embodiments, the pathogen is a virus, e.g. a Coronavirus (e.g. SARS-CoV-2) or a Rabies virus

In particularly preferred embodiments, the disorder an infection with a Coronavirus, or a disorder related to such infections, in particular an infection with SARS-CoV-2, or a disorder related to such infections (e.g. COVID-19).

In other embodiments, the disorder is a tumour disease or a disorder related to such tumour disease, a protein or enzyme deficiency, or a genetic disorder or condition.

In preferred embodiments, the present invention relates to a method of treating or preventing a disorder as defined above, wherein the method comprises applying or administering to a subject in need thereof the pharmaceutical composition for multidose administration of the first aspect, the vaccine for multidose administration of the second aspect, or the kit or kit of parts of the third aspect.

In particular, the method treating or preventing a disorder may comprise the steps of:
a) providing the pharmaceutical composition for multidose administration of the first aspect, the vaccine for multidose administration of the second aspect, or the kit or kit of parts of the third aspect;
b) applying or administering said composition, vaccine, or kit or kit of parts to a subject as a first dose;
c) optionally, applying or administering said composition, vaccine, or kit or kit of parts to a subject as a second dose or a further dose.

### Method of Formulating a Multidose Composition or Vaccine

In a further aspect, the present invention provides a method of formulating a multidose composition or vaccine.

It has to be noted that features and embodiments that are described in the context of the pharmaceutical composition for multidose administration of the first aspect or the vaccine for multidose administration of the second aspect or the kit or kit of parts of the third aspect may also be applicable to the method of formulating a multidose composition or vaccine.

Likewise, features and embodiments that are described in the context of the method of formulating a multidose composition or vaccine may also be applicable to the composition, the vaccine, or the kit or kit of parts.

In preferred embodiments, the method of formulating a multidose composition or vaccine comprises the steps
a) obtaining a first component comprising lipid-based carriers encapsulating an RNA;
b) obtaining a second component comprising at least one antimicrobial preservative preferably selected from at least one aromatic alcohol, at least one sugar alcohol, thiomersal, or a combination thereof, preferably at least one aromatic alcohol; and
c) mixing said first and second components to formulate a multidose composition or vaccine.

In preferred embodiments, the first component is a liquid composition.

In alternative embodiments, the first component is a lyophilized or spray-dried or spray-freeze dried composition.

In preferred embodiments, the second component is a sterile liquid obtained by a step of heat sterilization.

In preferred embodiments, the lipid-based carriers of the first component are as defined in the context of the first aspect.

Accordingly, in preferred embodiments, the lipid-based carrier comprises
i. at least one cationic lipid, preferably as defined in the context of the first aspect;
ii. at least one neutral lipid, preferably as defined in the context of the first aspect;
iii. at least one steroid or steroid analogue, preferably as defined in the context of the first aspect; and iv. at least one aggregation reducing lipid, preferably as defined in the context of the first aspect.

Suitably, (i) to (iv) are in a molar ratio of about 20-60% cationic lipid, about 5-25% neutral lipid, about 25-55% steroid or steroid analog, and about 0.5-15% aggregation reducing lipid.

In preferred embodiments, the RNA of the first component is as defined in the context of the first aspect.

In embodiments, the first component is provided by according to the general procedures described in PCT Pub. Nos. WO 2015/199952, WO 2017/004143 and WO 2017/075531, the full disclosures of which are incorporated herein by reference.

Essentially, an aqueous RNA solution and an ethanolic lipid solution may be combined with certain flow rates to allow the formation of lipid-based carriers encapsulating an RNA. In short, lipid nanoparticles (LNP) may be prepared at a ratio of mRNA to total Lipid of 0.03-0.04 w/w. Pumps may be used to combine the ethanolic lipid solution with a flow rate F1 and the mRNA aqueous solution with a flow rate F2 at a ratio of about 1:5 to 1:3 (vol/vol) in a T-piece system. F1 and/or F2 may be adjusted to flow rates above 15 ml/min to allow the formation of LNPs encapsulating the RNA that have a Z-average size in a range from about 60 nm to about 115 nm. After formulation, the ethanol may be removed by at least one TFF step and at least one clarifying filtration step. After clarifying filtration, the filtrate may be adjusted to a desired concentration using a storage buffer comprising 150 mM sucrose, 75 mM sodium chloride, 10 mM sodium phosphate, pH 7.4. Subsequently, the resulting formulation may be filtered through sterilizing filters to reduce bioburden.

In preferred embodiments, the ethanolic lipid solution is prepared by solubilizing lipids in a certain molar ratio in ethanol. In preferred embodiments, a cationic lipid according to formula III-3, DSPC, cholesterol, and the aggregation reducing lipid, e.g. according to formula IVa (n=49 or n=45) is solubilized in ethanol at a certain molar ratio, preferably at a molar ratio of approximately 47.5:10:40.8:1.7 or 47.4:10:40.9:1.7.

In preferred embodiments, the aqueous RNA solution is prepared by adjusting RNA to a certain concentration, e.g. a concentration of about 0.2 mg/mL in e.g. a citrate or acetate buffer (preferably citrate buffer).

In preferred embodiments, the antimicrobial preservative of the second component is as defined in the first aspect.

In preferred embodiments, the antimicrobial preservative of the second component is at least one aromatic alcohol.

In particularly preferred embodiments, the at least one aromatic alcohol of the second component is phenoxyethanol.

In preferred embodiments, the at least one sugar alcohol of the second component is selected from xylitol, sorbitol, and/or glycerol, preferably xylitol.

In preferred embodiments, the method is a method of formulating a multidose composition of the first aspect. Accordingly, the formulated multidose composition is a composition as defined in the context of the first aspect.

In preferred embodiments, the method is a method of formulating a multidose vaccine of the second aspect. Accordingly, the formulated multidose vaccine is a vaccine as defined in the context of the second aspect.

The invention also relates to a multidose composition or vaccine formulated by or obtainable by the method of formulating a multidose composition or vaccine

### Uses

Also provided herein are various uses as specified herein of antimicrobial preservatives as specified herein.

According to further aspects, the invention relates to the use of certain particularly suitable antimicrobial preservatives in preserving and/or formulating a composition or vaccine comprising lipid-based carriers encapsulating RNA.

Notably, embodiments relating to the composition of the first aspect, the vaccine of the second aspect, or the kit or kit of parts of the third aspect may likewise be read on and be understood as suitable embodiments of uses as provided herein.

Further, embodiments relating to the method of formulating as provided above may likewise be read on and be understood as suitable embodiments of uses as provided herein, in particular for uses for formulating a composition or vaccine.

In certain aspects, the invention relates to the use of aromatic alcohols for preserving and/or formulating a composition or vaccine comprising lipid-based carriers encapsulating an RNA. In preferred embodiments of the use, suitable aromatic alcohols are selected from phenylethyl alcohol, phenoxyethanol, benzyl alcohol, or combinations thereof. In particularly preferred embodiments of the use, the aromatic alcohol is selected from phenoxyethanol.

In certain aspects, the invention relates to the use of sugar alcohol for preserving and/or formulating a composition or vaccine comprising lipid-based carriers encapsulating an RNA. In preferred embodiments of the use, suitable sugar alcohol are selected from xylitol, sorbitol, glycerol, or combinations thereof. In preferred embodiments of the use, the sugar alcohol is selected from xylitol.

In certain aspects, the invention relates to the use of thiomersal for preserving and/or formulating a composition or vaccine comprising lipid-based carriers encapsulating an RNA.

Unexpectedly, as shown herein, antimicrobial preservatives that comprises a phenol or a phenol group (e.g. chlorocresol, chloroxylenol, cresol, or phenol) are not suitable for preserving and/or preparing a composition or vaccine comprising lipid-based carriers encapsulating an RNA.

In certain aspects, the invention relates to the use of phenol-free antimicrobial preservatives for preserving and/or preparing a composition or vaccine comprising lipid-based carriers encapsulating an RNA. In preferred embodiments of the use, suitable phenol-free antimicrobial preservatives are selected from benzyl alcohol, bronopol, cetrimide, cetylpyridinium chloride, chlorhexidine, chlorobutanol, ethyl alcohol, hexetidine, imidurea, phenoxyethanol, phenylethyl alcohol, phenylmercuric nitrate, propylene glycol, xylitol, sorbitol, glycerol, and/or thimerosal.

Method of Expressing a Peptide or Protein Encoded by an mRNA in a Cell or a Subject

In another aspect, the present invention relates to a method of expressing a peptide or protein encoded by an mRNA in a cell or a subject.

In preferred embodiments, the method of expressing a peptide or protein encoded by an mRNA in a cell or a subject comprises the steps of
(i) obtaining the pharmaceutical composition for multidose administration of the first aspect, the vaccine for multidose administration of the second aspect, or the kit or kit of parts of the third aspect; and
(ii) administering an effective amount of the composition and/or the vaccine and/or the kit or kit of parts to a cell or a subject.

In a preferred embodiment, the liquid composition and/or vaccine as defined herein has been stored as a liquid after formulation and/or a first withdrawal, preferably wherein storage is at a temperature above freezing temperature, preferably at about 5° C. since formulation. In embodiments, the composition or vaccine as defined herein has been stored as a liquid at a temperature in a range from about 1° C. to about 25° C.

Notably, multiple withdrawals from the composition or vaccine as defined herein does not reduce the amount of peptide or protein that is produced in said cell or said subject upon administration of the mRNA formulated in the lipid-based carrier. In embodiments, multiple withdrawals from the composition or vaccine as defined herein reduces the amount of peptide or protein that is produced in said cell or said subject by not more than 30%, 20%, 15%, 10%, or 5%. “The amount of peptide or protein” that is produced in said cell or said subject has to be understood as the product of translation of the mRNA that is administered. The amount of peptide or protein may be determined by standard techniques including but not limited to analyzing a sample obtained from the cell or subject using western blot, ELISA, or mass spectrometry.

### LIST OF PARTICULARLY PREFERRED EMBODIMENTS (ITEMS)

Item 1. A pharmaceutical composition for multidose administration comprising lipid-based carriers encapsulating an RNA, wherein the composition comprises at least one antimicrobial preservative selected from at least one aromatic alcohol, at least one sugar alcohol, thiomersal, or a combination thereof.

Item 2. The pharmaceutical composition for multidose administration of Item 1, wherein the at least one aromatic alcohol is selected from phenoxyethanol, phenylethyl alcohol, benzyl alcohol, or a combination thereof.

Item 3. The pharmaceutical composition for multidose administration of Item 1 to 2, wherein the at least one aromatic alcohol is phenoxyethanol.

Item 4. The pharmaceutical composition for multidose administration of item 1 to 2, wherein the at least one aromatic alcohol is benzyl alcohol.

Item 5. The pharmaceutical composition for multidose administration of any one of the preceding items, wherein the at least one aromatic alcohol is in a concentration of 0.1% (w/v) to 2% (w/v).

Item 6. The pharmaceutical composition for multidose administration of any one of the preceding items, wherein thiomersal is in a concentration of 0.0005% (w/v) to 0.05% (w/v).

Item 7. The pharmaceutical composition for multidose administration of any one of the preceding items, wherein the at least one sugar alcohol is selected from xylitol, sorbitol, and/or glycerol.

Item 8. The pharmaceutical composition for multidose administration of any one of the preceding items, wherein the at least one sugar alcohol is xylitol.

Item 9. The pharmaceutical composition for multidose administration of any one of the preceding items, wherein the at least one sugar alcohol in a concentration of about 10 mM to about 200 mM.

Item 10. The pharmaceutical composition for multidose administration of any one of the preceding items, wherein the composition comprises at least two antimicrobial preservatives selected from at least one aromatic alcohol and from at least one sugar alcohol.

Item 11. The pharmaceutical composition for multidose administration of item 10, wherein the at least one aromatic alcohol is phenoxyethanol and the least one sugar alcohol is xylitol.

Item 12. The pharmaceutical composition for multidose administration of any one of the preceding items, wherein the RNA has an RNA integrity of at least about 50%, preferably of at least about 60%, more preferably of at least about 70%, most preferably of at least about 80%.

Item 13. The pharmaceutical composition for multidose administration of any one of the preceding items, wherein the composition comprises less than about 20% free RNA, preferably less than about 15% free RNA, more preferably less than about 10% free RNA.

Item 14. The pharmaceutical composition for multidose administration of any one of the preceding items, wherein the RNA has a length ranging from about 200 nucleotides to about 10000 nucleotides, preferably wherein the RNA is at least 500 nt in length Item 15. The pharmaceutical composition for multidose administration of any one of the preceding items, wherein the RNA comprises at least one coding sequence.

Item 16. The pharmaceutical composition for multidose administration of item 15, wherein the coding sequence encodes at least one peptide or protein suitable for use in treatment or prevention of a disease, disorder or condition.

Item 17. The pharmaceutical composition for multidose administration of item 16, wherein the at least one peptide or protein is selected or derived from an antigen or epitope of a pathogen, preferably selected or derived from a Coronavirus or a Rabies virus, or a fragment or variant of any of these.

Item 18. The pharmaceutical composition for multidose administration of any one of the preceding items, wherein the RNA comprises a 5′ cap structure, preferably a cap1 structure.

Item 19. The pharmaceutical composition for multidose administration of any one of the preceding items, wherein the RNA is an mRNA.

Item 20. The pharmaceutical composition for multidose administration of any one of the preceding items, wherein the wt/wt ratio of lipid to the RNA is from about 10:1 to about 60:1, preferably from about 20:1 to about 30:1, more preferably about 25:1.

Item 21. The pharmaceutical composition for multidose administration of any one of the preceding items, wherein the N/P ratio of the lipid-based carriers to the RNA is in a range from about 1 to about 10, preferably in a range from about 5 to about 7, more preferably about 6.

Item 22. The pharmaceutical composition for multidose administration of any one of the preceding items, wherein the lipid-based carriers have a polydispersity index (PDI) value of less than about 0.3, preferably of less than about 0.2, more preferably of less than about 0.1.

Item 23. The pharmaceutical composition for multidose administration of any one of the preceding items, wherein the lipid-based carriers have a Z-average size in a range from about 50 nm to about 150 nm, preferably in a range from about 50 nm to about 120 nm, more preferably in a range of about 60 nm to about 115 nm Item 24. The pharmaceutical composition for multidose administration of any one of the preceding items, wherein the lipid-based carriers are liposomes, lipid nanoparticles, lipoplexes, and/or nanoliposomes.

Item 25. The pharmaceutical composition for multidose administration of any one of the preceding items, wherein the lipid-based carriers are lipid nanoparticles.

Item 26. The pharmaceutical composition for multidose administration of any one of the preceding items, wherein the lipid-based carriers comprise at least one aggregation-reducing lipid, at least one cationic lipid, at least one neutral lipid, and/or at least one steroid or steroid analog.

Item 27. The pharmaceutical composition for multidose administration of item 26, wherein the aggregation reducing lipid is a polymer conjugated lipid, e.g. a PEG-conjugated lipid.

Item 28. The pharmaceutical composition for multidose administration of item 27, wherein the polymer conjugated lipid is a PEG-conjugated lipid according to formula (IVa):

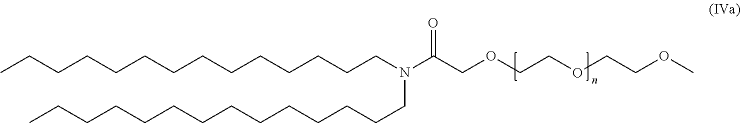

wherein n has a mean value ranging from 30 to 60, preferably wherein n has a mean value of about 45, 46, 47, 48, 49, 50, 51, 52, 53, 54, more preferably wherein n has a mean value of 49 or 45.

Item 29. The pharmaceutical composition for multidose administration of item 26 to 28, wherein the at least one cationic lipid is selected from a lipid according to formula III-3:

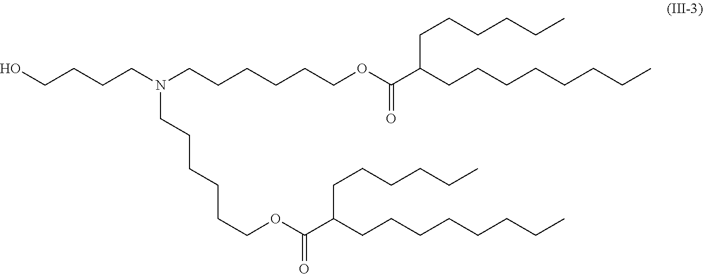 [SMILES: CCCCCCCCC(CCCCCC)C(=O)OCCCCCCN(CCCCO)CCCCCCOC(=O)C(CCCCCC)CCCCCCCC]

Item 30. The pharmaceutical composition for multidose administration of items 26 to 29, wherein the at least one neutral lipid is 1,2-distearoyl-sn-glycero-3-phosphocholine (DSPC).

Item 31. The pharmaceutical composition for multidose administration of items 26 to 30, wherein the steroid or steroid analog is cholesterol.

Item 32. The pharmaceutical composition for multidose administration of any one of the preceding items, wherein the lipid-based carrier comprises
i. at least one cationic lipid, preferably as defined in item 29;
ii. at least one neutral lipid, preferably as defined in item 30;
iii. at least one steroid or steroid analogue, preferably as defined in item 31; and
iv. at least one aggregation reducing lipid, preferably as defined in item 27 or 28.

Item 33. The pharmaceutical composition for multidose administration of item 32, wherein (i) to (iv) are in a molar ratio of about 20-60% cationic lipid, about 5-25% neutral lipid, about 25-55% steroid or steroid analog, and about 0.5-15% aggregation reducing lipid.

Item 34. The pharmaceutical composition for multidose administration of item 32 or 33, wherein (i) to (iv) are in a molar ratio of about 47.4% cationic lipid, 10% neutral lipid, 40.9% steroid or steroid analogue, and 1.7% aggregation reducing lipid.

Item 35. The pharmaceutical composition for multidose administration of any one of the preceding items, further comprising a sugar in a concentration of about 5 mM to about 300 mM, preferably sucrose in a concentration of about 14 mM.

Item 36. The pharmaceutical composition for multidose administration of any one of the preceding items, further comprising a salt in a concentration of about 10 mM to about 300 mM, preferably NaCl in a concentration of about 150 mM.

Item 37. The pharmaceutical composition for multidose administration of any one of the preceding items, wherein the composition is free of virus particles and/or wherein the composition does not comprise and added adjuvant.

Item 38. The pharmaceutical composition for multidose administration of any one of the preceding items, wherein the composition has been formulated by adding the at least one antimicrobial preservative to a composition comprising lipid-based carriers encapsulating an RNA.

Item 39. The pharmaceutical composition for multidose administration of item 38, wherein the composition is stable for at least about 1 day after formulation of the composition.

Item 40. The pharmaceutical composition for multidose administration of any one of the preceding items, wherein the composition is stable for at least about 1 day after a first dose withdrawal.

Item 41. The pharmaceutical composition for multidose administration of any one of the preceding items, wherein after a first dose withdrawal and/or after formulation of the composition, the integrity of the RNA decreases less than about 30%, preferably less than about 20%, more preferably less than about 10%.

Item 42. The pharmaceutical composition for multidose administration of any one of the preceding items, wherein after a first dose withdrawal and/or after formulation of the composition, the amount of free RNA does not increase by more than 10%, preferably by not more than 5%.

Item 43. The pharmaceutical composition for multidose administration of any one of the preceding items, wherein after a first dose withdrawal and/or after formulation of the composition, the PDI value of the lipid-based carriers encapsulating the RNA does not increase by more than a value of about 0.2, preferably by not more than a value of about 0.1.

Item 44. The pharmaceutical composition for multidose administration of any one of the preceding items, wherein after a first dose withdrawal and/or after formulation of the composition, the Z-average size of the lipid-based carriers encapsulating the RNA does not increase by more than 20%, preferably by not more than 10%.

Item 45. The pharmaceutical composition for multidose administration of any one of the preceding items, wherein the composition is microbially preserved for at least about 1 day.

Item 46. The pharmaceutical composition for multidose administration of any one of the preceding items, wherein the composition is microbially preserved at a temperature of about 5° C. to about 25° C.

Item 47. The pharmaceutical composition for multidose administration of any one of the preceding items, wherein the composition is microbially preserved for at least about 1 day after a first dose withdrawal, Item 48. A vaccine for multidose administration comprising or consisting of a pharmaceutical composition for multidose administration of any one of items 1 to 47.

Item 49. The vaccine for multidose administration of item 48, wherein the vaccine is against a Coronavirus, preferably against SARS-CoV-2.

Item 50. The vaccine for multidose administration of item 48 or 49, wherein the vaccine is against a pandemic virus.

Item 51. A kit or kit of parts for preparing and/or administering a multidose composition or vaccine wherein the kit comprises the following components
(A) at least one pharmaceutical composition comprising lipid-based carriers encapsulating an RNA; and
(B) at least one sterile buffer for diluting component A, wherein the sterile dilution buffer comprises at least one antimicrobial preservative selected from at least one aromatic alcohol, at least one sugar alcohol, thiomersal, or a combination thereof.

Item 52. The Kit or kit of parts of item 51, wherein the multidose composition or vaccine is a pharmaceutical composition for multidose administration as defined in items 1 to 47, or a vaccine for multidose administration as defined in items 48 to 50.

Item 53. The kit or kit of parts of item 51 or 52, wherein component A and component B are provided in separate containers or vials.

Item 54. The kit or kit of parts of items 51 to 53, wherein component A and component B are combined to obtain a diluted pharmaceutical composition or vaccine for multidose administration.

Item 55. The Kit or kit of parts of item 54, wherein the dilution factor is in a range from 1:1 to 1:50, preferably between 1:5 and 1:15.

Item 56. The kit or kit of parts of item 51 to 55, wherein component B comprises at least one antimicrobial preservative as defined in items 2 to 11.

Item 57. The kit or kit of parts of items 51 to 56, wherein component B comprises at least one aromatic alcohol, preferably phenoxyethanol and, optionally, at least one sugar alcohol, preferably xylitol.

Item 58. The kit or kit of parts of items 51 to 57, wherein component B comprises a salt, preferably NaCl, optionally in a concentration of about 0.9%.

Item 59. The kit or kit of parts of items 51 to 58, wherein component B is a heat autoclaved sterile buffer.

Item 60. The kit or kit of parts of items 51 to 59, wherein the lipid-based carriers of component A are as defined in items 20 to 34.

Item 61. The kit or kit of parts of items 51 to 60, wherein the RNA of component A is as defined in items 12 to 19.

Item 62. The kit or kit of parts of items 51 to 61, wherein component A comprises a sugar in a concentration of about 50 mM to about 300 mM, preferably sucrose in a concentration of about 150 mM.

Item 63. The kit or kit of parts of items 51 to 62, wherein component A comprises a salt in a concentration of about 10 mM to about 200 mM, preferably NaCl in a concentration of about 75 mM.

Item 64. The Kit or kit of parts of items 51 to 63, wherein the kit or kit of parts comprises at least one means for combining component A and component B to obtain the multidose composition or vaccine.

Item 65. Kit or kit of parts of items 51 to 64, wherein the kit or kit of parts comprises at least one means for administering the multidose composition or vaccine.

Item 66. Kit or kit of parts of items 51 to 65, wherein, after combining component A and component B, the integrity of the RNA decreases less than about 30%, preferably less than about 20%, more preferably less than about 10%.

Item 67. Kit or kit of parts of items 51 to 66, wherein, after combining component A and component B, the amount of free RNA does not increase by more than 10%, preferably by not more than 5%.

Item 68. Kit or kit of parts of items 51 to 67, wherein, after combining component A and component B, the PDI value of the lipid-based carriers encapsulating the RNA does not increase by more than a value of about 0.2, preferably by not more than a value of about 0.1.

Item 69. Kit or kit of parts of items 51 to 68, wherein, after combining component A and component B, the Z-average size of the lipid-based carriers encapsulating the RNA does not increase by more than 20%, preferably by not more than 10%.

Item 70. Kit or kit of parts of items 51 to 69, wherein, after combining component A and component B, the obtained composition or vaccine is microbially preserved, preferably for at least about 1 day and/or at a temperature of about 5° C. to about 25° C. and/or for at least about 1 day after a first dose withdrawal.

Item 71. The pharmaceutical composition for multidose administration of any one of items 1 to 47, the vaccine of items 48 to 50, the kit or kit of parts of any one of items 51 to 69, for use as a medicament.

Item 72. The pharmaceutical composition for multidose administration of any one of items 1 to 47, the vaccine of items 48 to 50, the kit or kit of parts of any one of items 51 to 69, for use in the treatment or prophylaxis of an infection with a pathogen or of a disorder related to such an infection, preferably wherein the pathogen is a Coronavirus.

Item 73. A method of treating or preventing a disorder, wherein the method comprises applying or administering to a subject in need thereof the pharmaceutical composition for multidose administration of any one of items 1 to 47, the vaccine of items 48 to 50, the kit or kit of parts of any one of items 51 to 69.

Item 74. A method of treating or preventing a disorder of item 73, wherein the disorder is an infection with a pathogen, preferably an infection with a Coronavirus.

Item 75. A method of formulating a multidose composition or vaccine comprising:
a) obtaining a first component comprising lipid-based carriers encapsulating an RNA;
b) obtaining a second component comprising at least one antimicrobial preservative selected from at least one aromatic alcohol, at least one sugar alcohol, thiomersal, or a combination thereof; and
c) mixing said first and second components to formulate a multidose composition or vaccine.

Item 76. The method of item 75, wherein the first component is a liquid composition.

Item 77. The method of items 75, wherein the first component is lyophilized or spray-dried composition.

Item 78. The method of items 75 to 77, wherein the lipid-based carriers of the first component are as defined in items 20 to 34.

Item 79. The method of items 75 to 78, wherein the RNA of the first component is as defined in items 12 to 19.

Item 80. The method of items 75 to 79, wherein the antimicrobial preservative of the second component are as defined in items 2 to 11.

Item 81. The method of items 75 to 80, wherein the multidose composition or vaccine is a composition or vaccine as defined in any one of items 1 to 47 or items 48 to 50.

Item 82. Use of an aromatic alcohol for preserving and/or formulating a composition or vaccine comprising lipid-based carriers encapsulating an RNA.

Item 83. Use of a sugar alcohol for preserving and/or formulating a composition or vaccine comprising lipid-based carriers encapsulating an RNA.

Item 84. Use of thiomersal for preserving and/or formulating a composition or vaccine comprising lipid-based carriers encapsulating an RNA.

### Brief Description of Lists and Tables

List 1: Suitable pathogens of the invention
Table 1: mRNA sequences used in the examples
Table 2: Lipid-based carrier composition of the examples
Table 3: Compounds tested in the preservative screening
Table 4: Formulation comprising lipid-based carriers encapsulating an RNA for preservative screening (Example 2)
Table 5: Formulations comprising lipid-based carriers encapsulating an RNA for preservative evaluation (Example 3)
Table 6: RNA integrity is not affected by aromatic alcohol preservatives
Table 7: Formulations comprising 2-PE used for the antimicrobial effectiveness study (Example 4)
Table 8: Results of the antimicrobial effectiveness study: Pseudomonas aeruginosa (Example 4)
Table 9: Results of the antimicrobial effectiveness study: Staphylococcus aureus (Example 4)
Table 10: Study design of the in vivo vaccination experiment (Example 5)

### BRIEF DESCRIPTION OF THE DRAWINGS

FIG. 1 shows an particle analysis performed using MFI (see Example 2). For each formulation (F1 to F11; see Table 4) different time points were analyzed, and for each timepoint/condition the respective particle concentration is shown in the following order: T0, T6 h_5° C., T6 h_25° C., T24_5° C., T48 h_5° C.; from left to right column).

FIG. 2 shows the Z-average size measurements performed using DLS (see Example 2). For each formulation (F1 to F11; see Table 4) different time points were analyzed, and each timepoint/condition the respective Z-average size is shown in the following order: TO, T6 h_5° C., T6 h_25° C., T24_5° C., T48 h_5° C.; from left to right column). For reference, a dashed line indicates 115 nm particle size.

FIG. 3 shows the RNA encapsulation in [%] for different formulations (see Example 2). For each formulation (F1 to F11; see Table 4) different time points were analyzed, and each timepoint/condition the respective encapsulation in [%] is shown in the following order: TO, T6 h_5° C., T6 h_25° C., T24_5° C., T48 h_5° C.; from left to right column).

FIG. 4 shows VNTs in serum samples taken on day 42. Each dot represents an individual animal; bars depict the median. For further details see Example 5.

### EXAMPLES

In the following, examples illustrating various embodiments and aspects of the invention are presented. However, the present invention shall not to be limited in scope by the specific embodiments presented herein, and should rather be understood as being applicable to other compositions and/or vaccines and/or uses as for example defined in the specification. Accordingly, the following preparations and examples are given to enable those skilled in the art to more clearly understand and to practice the present invention. Indeed, various modifications of the invention in addition to those described herein will become readily apparent to those skilled in the art from the foregoing description, accompanying figures and the examples below.

### Example 1: Preparation of Compositions Comprising Lipid-Based Carriers Encapsulating an RNA

The present example provides methods of obtaining the RNA of the invention as well as methods of generating a composition or a vaccine of the invention comprising lipid-based carriers encapsulating an RNA.

### 1.1 Preparation of DNA Templates for RNA In Vitro Transcription:

DNA sequences encoding a Coronavirus spike antigen (full length prefusion stabilized SARS-CoV-2 spike protein comprising K986P, V987P substitutions; S_stab) or Rabies virus glycoprotein were prepared and used for subsequent RNA in vitro transcription reactions. Said DNA sequences were prepared by modifying the wild type encoding DNA sequences by introducing a G/C optimized coding sequence for stabilization and expression optimization. Sequences were introduced into a pUC derived DNA vector to comprise a stabilizing 3′-UTR sequences and a stretch of adenosines (A64), a histone-stem-loop (hSL) structure and a stretch of 30 (C30) (see Table 1). The obtained plasmid DNA templates were transformed and propagated in bacteria using common protocols known in the art. Eventually, the plasmid DNA templates were extracted, purified, and used for linearization reaction using EcoRI as digestion enzyme.

TABLE 1
mRNA used in Examples

| | | SEQ ID NO: | SEQ ID NO: | SEQ ID NO: |
|---|---|---|---|---|
| RNA ID | Name | Protein | CDS | mRNA |
| R9515 | SARS-CoV-2 Spike | SEQ ID NO: 1 | SEQ ID NO: 2 | SEQ ID NO: 3 |
| | protein | | | |
| R1803 | Rabies virus | SEQ ID NO: 4 | SEQ ID NO: 5 | SEQ ID NO: 6 |

1.3. RNA In Vitro Transcription from Plasmid DNA Templates:

A linearized DNA template encoding R9515 was used for DNA dependent RNA in vitro transcription using T7 RNA polymerase in the presence of a sequence optimized nucleotide mixture (ATP/GTP/CTP/UTP) and cap analog (for Cap1: m7G(5′)ppp(5′)(2′OMeA)pG) under suitable buffer conditions. After RNA in vitro transcription, the obtained RNA IVT reaction comprising the mRNA was subjected to purification steps comprising TFF and RP-HPLC.

A linearized DNA template encoding R1803 was used for DNA dependent RNA in vitro transcription using T7 RNA polymerase in the presence of a nucleotide mixture (ATP/GTP/CTP/UTP) and cap analog (Cap0: m7GpppG) under suitable buffer conditions. After RNA in vitro transcription, the obtained RNA IVT reaction comprising the mRNA was subjected to purification steps comprising TFF and RP-HPLC.

1.4. Preparation of Lipid-Based Carriers (LNP) Encapsulating the mRNA:

An ethanolic lipid solution was prepared by solubilizing the cationic lipid according to formula III-3, DSPC, cholesterol, and the aggregation reducing lipid (PEG-conjugated lipid) according to formula IVa (n=49 or n=45) in ethanol at a molar ratio of approximately 47.5:10:40.8:1.7 or 47.4:10:40.9:1.7. (see Table 2).

TABLE 2
Lipid-based carrier composition of the examples

| | | Ratio | | |
|---|---|---|---|---|
| | Compounds | (mol %) | Structure | Mass |
| 1 | Cholesterol | 40.9 | 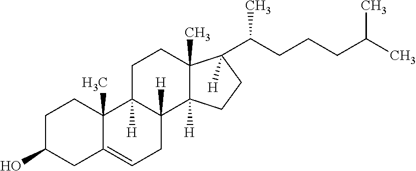 | 386.4 |
| 2 | 1,2- distearoyl- sn- glycero-3- phos- phocholine (DSPC) | 10 | 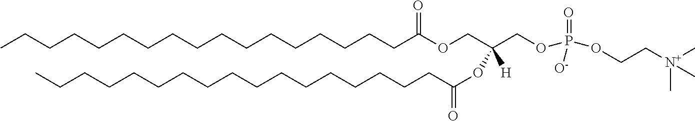 [SMILES: CCCCCCCCCCCCCCCCCC(=O)OC[C@H](COP(=O)([O-])OCC[N+](C)(C)C)OC(=O)CCCCCCCCCCCCCCCCC] | 789.6 |
| 3 | Cationic Lipid | 47.4 | 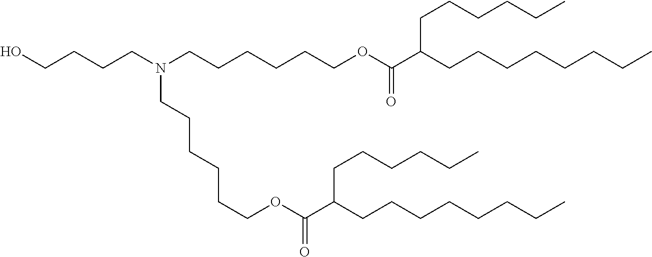 [SMILES: CCCCCCCCC(CCCCCC)C(=O)OCCCCCCN(CCCCO)CCCCCCOC(=O)C(CCCCCC)CCCCCCCC] | 765.7 |
| 4 | PEG Lipid | 1.7 | 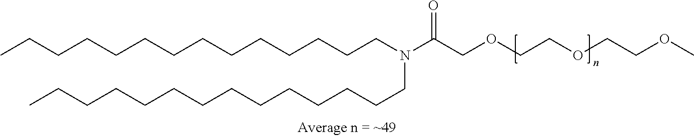 | 2010.1 |

An aqueous RNA solution was prepared by adjusting the RNA (obtained according to Example 1.3) to a concentration of about 0.2 mg/mL in 50 mM citrate buffer, pH 4.

Lipid nanoparticles were prepared according to the general procedures described in PCT Pub. Nos. WO 2015/199952, WO 2017/004143 and WO 2017/075531, the full disclosures of which are incorporated herein by reference.

In short, lipid nanoparticles (LNP) were prepared at a ratio of mRNA to total Lipid of 0.03-0.04 w/w. Pumps were used to combine the ethanolic lipid solution with a flow rate F1 and the mRNA aqueous solution with a flow rate F2 at a ratio of about 1:5 to 1:3 (vol/vol) in a T-piece system, F1 and/or F2 were adjusted to flow rates above 15 ml/min to allow the formation of LNPs encapsulating the RNA that have a Z-average size in a range from about 60 nm to about 115 nm. After formulation, the ethanol was removed by at least one TFF step and at least one clarifying filtration step. After clarifying filtration, the filtrate was adjusted to a desired concentration (typically 1 g/l RNA) using a storage buffer comprising 150 mM sucrose, 75 mM sodium chloride, 10 mM sodium phosphate, pH 7.4. Subsequently, the resulting formulation was filtered through sterilizing filters to reduce bioburden. The formulation comprising lipid-based carriers encapsulating RNA was used for antimicrobial preservative screenings (see Examples below).

### Example 2: Screening of Different Formulation Comprising Antimicrobial Preservative Candidates

Formulations comprising lipid-based carriers (RNA sequence R1803; Rabies, see Table 1) were generated according to Example 1 and adjusted to a concentration of 0.25 mg/ml RNA using the storage buffer (10 mM sodium phosphate (pH 7.4), 75 mM NaCl, 150 mM sucrose) to obtain a stock formulation (“stock” in Table 3).

The different formulations were prepared by diluting the stock formulation using a 0.9% (w/v) NaCl solutions containing the different antimicrobial preservatives (see Table 3). The obtained formulations are listed in Table 4 (F1 to F11). The formulation F12 was used as a control an did not comprise an antimicrobial preservative.

TABLE 3
Compounds tested in the preservative screening

| Name | Manufacturer |
|---|---|
| 2-Phenoxyethanol 99.0-100.5% Ph. Eur. (2-PE) | VWR Chemicals |
| Phenol crystalline, Ph. Eur. | AppliChem |
| Thiomersal Ph Eur, BP | Sigma Aldrich |
| Benzyl alcohol EMPROVE ® EXPERT | Merck |
| Ph Eur, BP, JP, NF | |
| m-Cresol, Ph Eur. | Lanxess |
| Benzalkonium chloride (50% aqueous solution) | Caelo |
| EMPROVE ESSENTIAL Ph. Eur., NF | |

TABLE 4
Formulation comprising lipid-based carriers encapsulating an RNA for preservative screening

| | mRNA | Total lipid | | |
|---|---|---|---|---|
| | conc. | conc. | | |
| | [mg/ml] | [mg/ml] | antimicrobial preservative | Buffer |
| stock | 0.25 | 6.35 | none | 10 mM sodium phosphate, 75 mM NaCl, |
| | | | | 150 mM Sucrose, pH 7.4 |
| F1 | 0.025 | 0.635 | 0.3% (w/v) phenol | 1 mM sodium phosphate, 146 mM NaCl, |
| | | | | 15 mM Sucrose, pH 7.4 |
| F2 | 0.025 | 0.635 | 0.5% (w/v) phenol | 1 mM sodium phosphate, 146 mM NaCl, |
| | | | | 15 mM Sucrose, pH 7.4 |
| F3 | 0.025 | 0.635 | 0.1% (w/v) phenol + | 1 mM sodium phosphate, 146 mM NaCl, |
| | | | 0.2% (w/v) m-cresol | 15 mM Sucrose, pH 7.4 |
| F4 | 0.025 | 0.635 | 0.3% (w/v) phenoxyethanol | 1 mM sodium phosphate, 146 mM NaCl, |
| | | | | 15 mM Sucrose, pH 7.4 |
| F5 | 0.025 | 0.635 | 0.5% (w/v) phenoxyethanol | 1 mM sodium phosphate, 146 mM NaCl, |
| | | | | 15 mM Sucrose, pH 7.4 |
| F6 | 0.025 | 0.635 | 1% (w/v) phenoxyethanol | 1 mM sodium phosphate, 146 mM NaCl, |
| | | | | 15 mM Sucrose, pH 7.4 |
| F7 | 0.025 | 0.635 | 0.5% (w/v) benzyl alcohol | 1 mM sodium phosphate, 146 mM NaCl, |
| | | | | 15 mM Sucrose, pH 7.4 |
| F8 | 0.025 | 0.635 | 1% (w/v) benzyl alcohol | 1 mM sodium phosphate, 146 mM NaCl, |
| | | | | 15 mM Sucrose, pH 7.4 |
| F9 | 0.025 | 0.635 | 0.5% (w/v) benzyl alcohol + | 1 mM sodium phosphate, 146 mM NaCl, |
| | | | 0.005% (w/v) benzalkonium chloride | 15 mM Sucrose, pH 7.4 |
| F10 | 0.025 | 0.635 | 0.001% (w/v) thiomersal | 1 mM sodium phosphate, 146 mM NaCl, |
| | | | | 15 mM Sucrose, pH 7.4 |
| F11 | 0.025 | 0.635 | 0.01% (w/v) thiomersal | 1 mM sodium phosphate, 146 mM NaCl, |
| | | | | 15 mM Sucrose, pH 7.4 |
| F12 | 0.025 | 0.635 | No preservative | 1 mM sodium phosphate, 146 mM NaCl, |
| | | | | 15 mM Sucrose, pH 7.4 |

Following formulation, F1 to F12 were stored for up to 48 h in 6R vials (each containing 2.5 ml of sample) at 200 to 8° C. (Liebherr, Mediline LKPV 6520). Furthermore, the formulations F1 to F12 were stored for 6 h in 6R vials (each containing 2.5 ml of sample) at 25° C./60% r.h. (climate chamber, Konstant Klimaschrank HPP108, Memmert).

2.1. Determination of the z-Average Size of Lipid-Based Carriers Encapsulating the RNA

DLS measurements were conducted in a 96-well plate (Corning Costar) by using a Zetasizer APS 2000 plate reader (Malvern Instruments) instrument. Three wells (n=3, each with 200 μl of the sample) were analyzed at a temperature of 22° C. by using the automatic detection mode at an angle of 90° (backscattering). All samples were analyzed without dilution. The Malvern Zetasizer Software (version 7.03) was used to fit the autocorrelation function and to calculate Z-average diameter, polydispersity index (PDI), and particle size distribution (by intensity). F1 to F12 were measured using RI value of 1.45 and viscosity dispersant value of 0.9675 mPa*sec.

### 2.2. Determination of the RNA Encapsulation

A RiboGreen assay was used to determine the RNA encapsulation in the different formulations, carried out by using a Neo2sm plate reader (BioTek). The RiboGreen assay was performed according to the manufacturer's instructions.

### 2.3. Determination of Sub-Visible Particles by Micro-Flow Imaging (MFI)

MFI measurements were conducted with a MFI-5200 particle analyzer system (ProteinSimple) equipped with a silane-coated high-resolution 100-μm SP3 flow cell. The system was flushed with water and the background illumination was subsequently optimized by using formulation buffer. All mRNA LNP formulations (F1 to F12) were analyzed 10-fold diluted in the respective diluent. A pre-run volume of 0.20 ml was followed by a sample run of 0.28 ml. Approximately 1,100 images were taken per sample. Between the measurements, the flow cell was cleaned with water. MFI View System Software (MVSS) version 2-R2-6.1.20.1915 was used to perform the measurements and MFI View Analysis Suite (MVAS) software version 1.3.0.1007 was used to analyze the samples. Particle concentrations were corrected for the dilution factor.

### 2.4. Results

### Occurrence of Sub-Visible Particles

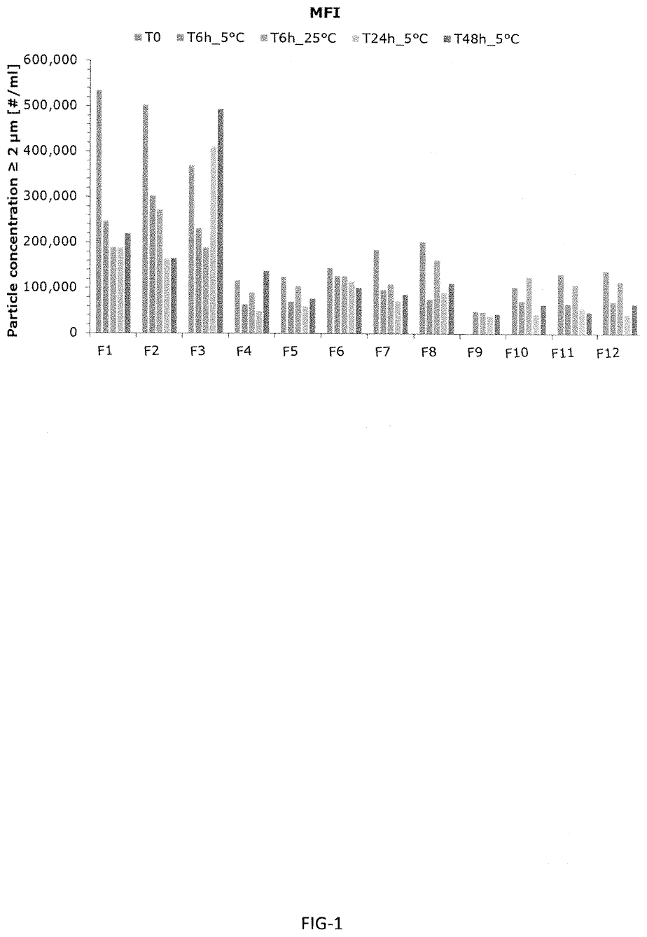

The occurrence of large sub-visible particles is not desired in compositions comprising lipid-based carrier encapsulating an RNA. The occurrence of such particles is indicating that the physiochemical properties of the lipid-based carrier encapsulating the RNA is affected by the added antimicrobial preservative. MFI measurements of the different formulations F1 to F12 were conducted as described in section 2.3 to determine sub-visible particles. Particles were analyzed at different time points after formulation and at certain storage conditions (0 h, 6 h at 5° C., 6 h at 25° C., 24 h at 5° C., 48 h at 5° C.). The results of the cumulative particle concentrations (#/ml) obtained by MFI analysis are summarized in FIG. 1.

At T0, the levels of sub visible particles 2 μm ranged between 100,000 to 500,000 #/ml. The highest levels of sub visible particle were observed in presence of phenol (F1, F2, F3). All other formulations demonstrated sub visible particle levels in a comparable range (comparable to the control F12), and without clear changes upon storage for up to 48 hours.

The results show that antimicrobial preservatives comprising phenol (e.g. F1, F2, F3) led to the formation of sub visible, and are therefore not suitable for multidose compositions or vaccines comprising lipid-based carriers encapsulating an RNA.

### Z-Average Particle Size:

Z-average particle size is an important quality attribute of compositions comprising lipid-based carrier encapsulating an RNA. A decrease or increase in particle size is indicating that the physiochemical properties of the lipid-based carrier encapsulating the RNA is affected by the added antimicrobial preservative. DLS measurements were conducted as described in section 2.1 to determine Z-average diameters (particle size) of the different formulations F1 to F12. Z-average particle sizes were analyzed at different time points after formulation at certain storage conditions (0 h, 6 h at 5° C., 6 h at 25° C., 24 h at 5° C., 48 h at 5° C.).

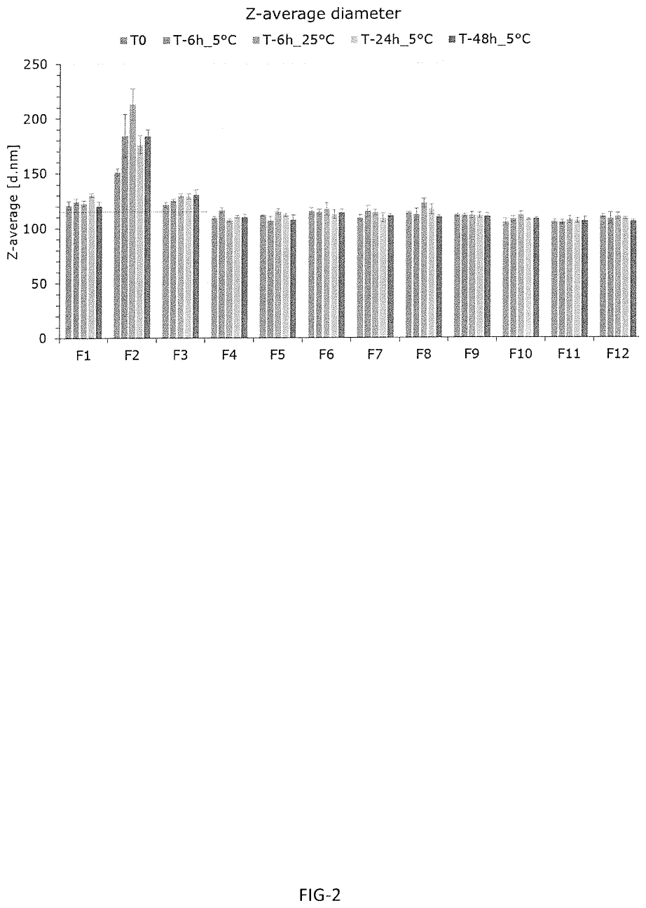

The results of the Z-average diameters (particle size) obtained by DLS analysis are summarized in FIG. 2.

As shown in FIG. 2, the Z-average diameters were ranging between 108 to 115 nm for most formulations (F4 to F11) with no distinguished differences to the control F12 irrespective of the storage conditions. An increased Z-average diameter (150 to 184 nm) compared to the control F12 was observed in F2 (0.5% phenol). Increased particle sizes compared to the control F12 were also observed for F1 and F3 (above 115 nm).

The results show that antimicrobial preservatives comprising phenol led to an increase in particle size, and are therefore not suitable for multidose compositions or vaccines comprising lipid-based carriers encapsulating an RNA.

### RNA Encapsulation:

RNA encapsulation is an important quality attribute of compositions comprising lipid-based carrier encapsulating an RNA. A decrease in RNA encapsulation is indicating that the physiochemical properties of the lipid-based carrier encapsulating the RNA is affected by the added antimicrobial preservative. A RiboGreen assay as commonly known in the art was performed (see section 2.2) to determine the RNA encapsulation % in the lipid based carriers of the different formulations F1 to F12. RNA encapsulation was analyzed at different time points after formulation and at certain storage conditions (0 h, 6 h at 5° C., 6 h at 25° C., 24 h at 5° C., 48 h at 5° C.). Prior to sample analysis, standard calibration curves were prepared to determine the amount of total RNA and free RNA present in the samples. All calibration curves showed sufficient correlation of R²≥0.99.

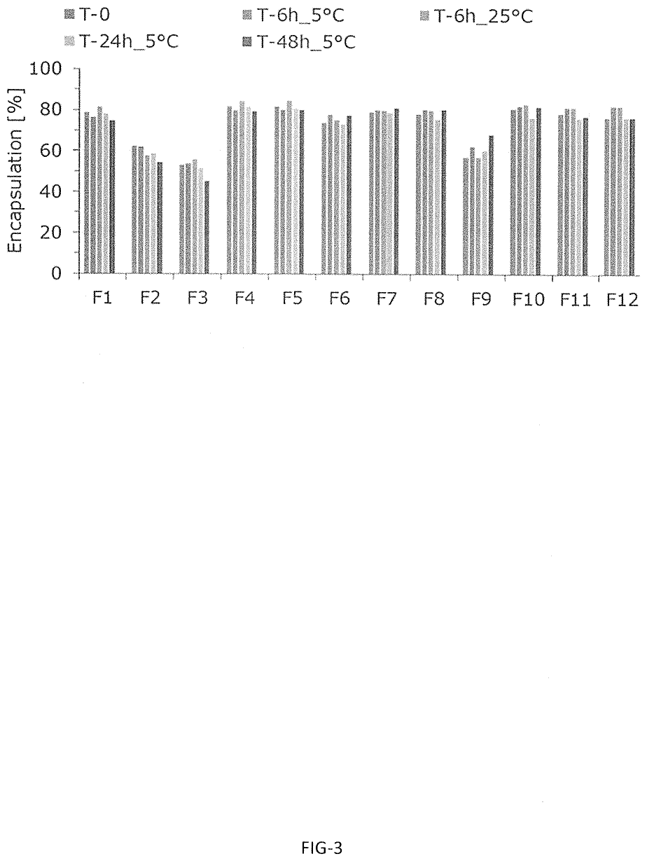

The results of the RiboGreen assay are summarized in FIG. 3.

As shown in FIG. 3, the percentage of encapsulated RNA was between 73% and 85% for formulations F1, F4, F5, F6, F7, F8, F10, and F11 with no distinguished differences to the control F12 irrespective of the storage conditions. Interestingly, a decrease in encapsulation efficiency was observed for F2 (0.5% phenol: ˜59%), F3 (phenol+m-cresol: ˜52%), and F9 (benzyl alcohol+benzalkonium chloride: ˜61%).

The results show that antimicrobial preservatives comprising phenol and antimicrobial preservatives comprising benzalkonium chloride led to a decrease in RNA encapsulation, and are therefore not suitable for multidose compositions or vaccines comprising lipid-based carriers encapsulating an RNA.

### Summary of the Results:

As shown in the present example, formulations comprising phenol, m-cresol, or benzalkonium chloride affected critical physiochemical attributes of the composition comprising lipid-based carriers encapsulating RNA. In particular, phenol, m-cresol, or benzalkonium chloride is leading to particle size increase, the formation of sub-visible particles, and a decrease in RNA encapsulation, showing that such antimicrobial preservatives are not suitable for compositions or vaccines for multidose of the invention.

The antimicrobial preservatives phenoxyethanol, benzyl alcohol, and thiomersal were identified to be compatible with compositions comprising lipid-based carriers encapsulating an RNA.

Selected lead candidate antimicrobial preservatives (aromatic alcohols phenoxyethanol, benzyl alcohol) were further evaluated, and the effect on the RNA integrity was tested (see Example 3).

### Example 3: Effect of Phenoxyethanol and Benzyl Alcohol on RNA Integrity

In the present example, the effect of two different aromatic alcohols on the RNA integrity was tested. As shown herein, aromatic alcohols do not have a negative impact on the RNA integrity of a composition comprising lipid-based carriers encapsulating an RNA (in the present example, the mRNA was encoding a SARS-CoV-2 antigen).

Formulations comprising lipid-based carriers encapsulating an RNA (R9515 encoding SARS-CoV-2; see Table 1) were generated according to Example 1 and adjusted to a concentration of 0.25 mg/ml RNA using the storage buffer (10 mM sodium phosphate (pH 7.4), 75 mM NaCl, 150 mM sucrose) to obtain a stock formulation (“stock”). The different formulations were prepared by diluting the stock formulation using a 0.9% (w/v) NaCl solutions containing the antimicrobial preservatives phenoxyethanol or benzyl alcohol. The obtained formulations are listed in Table 5 (F4, F5, F6, F8). Formulation F12 was used as a control an did not comprise an antimicrobial preservative.

TABLE 5
Formulations comprising lipid-based carriers encapsulating
an RNA for preservative evaluation (Example 3)

| | mRNA | Total lipid | | |
|---|---|---|---|---|
| | conc. | conc. | | |
| | [mg/ml] | [mg/ml] | antimicrobial preservative | Buffer |
| stock | 0.25 | 6.35 | none | 10 mM sodium phosphate, 75 mM NaCl, |
| | | | | 150 mM Sucrose, pH 7.4 |
| F4 | 0.025 | 0.635 | 0.3% (w/v) phenoxyethanol | 1 mM sodium phosphate, 146 mM NaCl, |
| | | | | 15 mM Sucrose, pH 7.4 |
| F5 | 0.025 | 0.635 | 0.5% (w/v) phenoxyethanol | 1 mM sodium phosphate, 146 mM NaCl, |
| | | | | 15 mM Sucrose, pH 7.4 |
| F6 | 0.025 | 0.635 | 1% (w/v) phenoxyethanol | 1 mM sodium phosphate, 146 mM NaCl, |
| | | | | 15 mM Sucrose, pH 7.4 |
| F8 | 0.025 | 0.635 | 1% (w/v) benzyl alcohol | 1 mM sodium phosphate, 146 mM NaCl, |
| | | | | 15 mM Sucrose, pH 7.4 |
| F12 | 0.025 | 0.635 | No preservative | 1 mM sodium phosphate, 146 mM NaCl, |
| | | | | 15 mM Sucrose, pH 7.4 |

### Determination of the RNA Integrity [%]

RNA integrity was determined using analytical (RP)HPLC. Samples of the liquid composition comprising the lipid based carrier encapsulating the RNA were treated with a detergent (about 2% Triton X100) to dissociate the lipid based carrier and to release the RNA. The released RNA was captured using Agencourt AMPure XP beads (Beckman Coulter, Brea, CA, USA) essentially according to the manufacturer's instructions. Following preparation of the RNA sample, analytical (RP)HPLC was performed to determine the integrity of the RNA. For determining the RNA integrity, the RNA sample was diluted to a concentration of 0.1 g/l using water for injection (WFI). 10 μl of the diluted RNA sample was injected into an HPLC column (a poly(styrene-divinylbenzene) matrix). Analytical (RP)HPLC was performed using standard conditions: Gradient 1: Buffer A (0.1 M TEAA (pH 7.0); Buffer B (0.1 M TEAA (pH 7.0) containing 25% acetonitrile. Starting at 30% buffer B the gradient extended to 32% buffer B in 2 min, followed by an extension to 55% buffer B over 15 minutes at a flow rate of 1 ml/min. HPLC chromatograms were recorded at a wavelength of 260 nm. The obtained chromatograms were evaluated using a software and the relative peak area was determined in percent (%). That value was used to assign an integrity value (RNA integrity [%]) to the test sample.

RNA integrity is an important quality attribute of compositions comprising lipid-based carrier encapsulating an RNA. A decrease in RNA integrity is indicating that the physiochemical properties of the RNA is affected by the added antimicrobial preservative, e.g. that the RNA is degraded or destroyed. RNA integrity measurements were performed as described above. RNA integrity was analyzed at different time points after formulation and at certain storage conditions (0 h, 6 h at 5° C., 24 h at 5° C., 48 h at 5° C.). The results are summarized in Table 6. As shown in Table 6, all tested formulations demonstrated RNA integrity levels in a comparable range (around 80%), and without clear changes upon storage for up to 48 hours.

TABLE 6
RNA integrity is not affected by aromatic alcohol preservatives

| | | | Determined |
|---|---|---|---|
| | | | integrity |
| Storage | | | value |
| condition | Formulation | Preservative | (HPLC) |
| 0 h | F4 | 0.3% (w/v) phenoxyethanol | 82% |
| 6 h at 5° C. | F4 | 0.3% (w/v) phenoxyethanol | 81% |
| 24 h at 5° C. | F4 | 0.3% (w/v) phenoxyethanol | 80% |
| 0 h | F5 | 0.5% (w/v) phenoxyethanol | 78% |
| 6 h at 5° C. | F5 | 0.5% (w/v) phenoxyethanol | 81% |
| 24 h at 5° C. | F5 | 0.5% (w/v) phenoxyethanol | 80% |
| 0 h | F6 | 1% (w/v) phenoxyethanol | 82% |
| 6 h at 5° C. | F6 | 1% (w/v) phenoxyethanol | 81% |
| 24 h at 5° C. | F6 | 1% (w/v) phenoxyethanol | 80% |
| 0 h | F8 | 1% (w/v) benzyl alcohol | 82% |
| 6 h at 5° C. | F8 | 1% (w/v) benzyl alcohol | 81% |
| 24 h at 5° C. | F8 | 1% (w/v) benzyl alcohol | 79% |
| 0 h | F12 | No preservative | 75% |
| 6 h at 5° C. | F12 | No preservative | 81% |
| 24 h at 5° C. | F12 | No preservative | 79% |

### Summary of the Results:

As shown in the present example, formulations comprising the aromatic alcohol preservatives phenoxyethanol or benzyl alcohol did not affect the RNA integrity. Moreover, as demonstrated in Example 2, the tested alcohol preservatives did also not affect quality attributes of the lipid-based carriers.

Accordingly, aromatic alcohol preservatives were identified to be compatible with compositions comprising lipid-based carriers encapsulating an RNA. As a next step, a selected aromatic alcohol preservative was tested in an antimicrobial effectiveness study (see Example 4).

### Example 4: Antimicrobial Effectiveness Study Using Phenoxyethanol (2-PE)

In the present example, the antimicrobial effectiveness of the aromatic alcohol 2-PE was analyzed. As shown herein, aromatic alcohols such as phenoxyethanol are microbially active in the presence of lipid-based carriers encapsulating an RNA.

Formulations comprising lipid-based carriers (RNA sequence R9515, SARS-CoV-2) were generated according to Example 1 and adjusted to a concentration of 0.25 mg/ml RNA using the storage buffer (10 mM sodium phosphate (pH 7.4), 75 mM NaCl, 150 mM sucrose) to obtain a stock formulation (“stock”). The different formulations were prepared by diluting the stock formulation using a 0.9% (w/v) NaCl solutions containing the antimicrobial preservatives phenoxyethanol. The obtained formulations are listed in Table 7 (F4, F5). Formulations F4 and F5 were subjected to an antimicrobial effectiveness study.

For testing the antimicrobial effectiveness of the aromatic alcohol 2-PE in the a composition comprising lipid-based carriers encapsulating an RNA, a procedure based on the European Pharmacopeia Ph Eur 5.1.3. (Efficacy of Antimicrobial Preservation) was conducted.

In short, the test consisted of challenging the respective formulation F4 and F5 with a prescribed inoculum of micro-organisms, storing the inoculated preparation at a prescribed temperature, withdrawing samples from the container at specified intervals of time, and counting the organisms in the samples so removed. As test microorganisms, Pseudomonas aeruginosa (ATCC 9027; NUIMB 8626; CIP 82.118) and Staphylococcus aureus (ATCC 6538; NCTC 10788; NCIMB 9518; CIP 4.83) was used. Different samples of formulations F4 and F5 were inoculated with a suspension of one of the test microorganisms Pseudomonas aeruginosa or Staphylococcus aureus to obtain an inoculum of 10\hat{ }5 to 10\hat{ }6 micro-organisms per mL. The inoculum was incubated at 20-25° C. for 0 h, 6 h, 24 h, 48 h, 7 days. After respective sampling time points (0 h, 6 h, 24 h, 48 h, 7 days), the number of viable micro-organisms was determined by plate count (in triplicates). For each condition, the log reduction of microorganisms compared to the inoculum was determined, e.g. a 1 log reduction stands for a 90% reduction of microorganisms, a 2 log reduction stands for a 99% reduction in microorganisms etc.

The results of the antimicrobial effectiveness tests are summarized in Table 8 (Pseudomonas aeruginosa) and Table 9 (Staphylococcus aureus).

TABLE 7
Formulations comprising 2-PE used for the antimicrobial effectiveness study (Example 4)

| | mRNA | Total lipid | | |
|---|---|---|---|---|
| | conc. | conc. | | |
| | [mg/ml] | [mg/ml] | antimicrobial preservative | Buffer |
| F5 | 0.025 | 0.635 | 0.5% (w/v) phenoxyethanol | 1 mM sodium phosphate, 146 mM NaCl, |
| | | | | 15 mM Sucrose, pH 7.4 |
| F6 | 0.025 | 0.635 | 1% (w/v) phenoxyethanol | 1 mM sodium phosphate, 146 mM NaCl, |
| | | | | 15 mM Sucrose, pH 7.4 |

TABLE 8
Results of the antimicrobial effectiveness study: Pseudomonas aeruginosa

| | | 0 h | 6 h | 24 h | 48 h | 7 d |
|---|---|---|---|---|---|---|
| | | [log reduction] | [log reduction] | [log reduction] | [log reduction] | [log reduction] |
| F5 | 0.5% 2-PE | 0 | 0.4 | 1.3 | 2.1 | 5.7 |
| | | 0 | 0.5 | 1.5 | 1.5 | 1.9 |
| | | 0 | 0.2 | 1.1 | 1.8 | 1.5 |
| F6 | 1% 2-PE | 0 | 4.7 | 5.7 | 5.7 | 5.7 |
| | | 0 | 4.9 | 5.9 | 5.9 | 5.9 |
| | | 0 | 4.5 | 5.5 | 5.5 | 5.5 |

TABLE 9
Results of the antimicrobial effectiveness study: Staphylococcus aureus

| | | 0 h | 6 h | 24 h | 48 h | 7 d |
|---|---|---|---|---|---|---|
| | | [log reduction] | [log reduction] | [log reduction] | [log reduction] | [log reduction] |
| F5 | 0.5% 2-PE | 0 | 0.2 | 0.0 | 0.1 | 1.7 |
| | | 0 | 0.1 | 0.1 | 0.2 | 1.5 |
| | | 0 | 0.1 | 0.0 | 0.2 | 1.8 |
| F6 | 1% 2-PE | 0 | 0.2 | 0.5 | 1.9 | 5.6 |
| | | 0 | 0 | 0.6 | 1.7 | 5.7 |
| | | 0 | 0.1 | 0.4 | 2.0 | 5.4 |

As shown in Table 8 (Pseudomonas aeruginosa) and Table 9 (Staphylococcus aureus), 2-PE has antimicrobiotic activity in the presence of lipid-based carriers encapsulating an RNA in the tested buffer system. Accordingly, the data clearly demonstrates that 2-PE is particularly suitable for preserving a multidose composition or vaccine comprising lipid-based carriers encapsulating an RNA.

### Summary of the Findings:

As shown in Example 2 and 3, the antimicrobial preservatives phenoxyethanol, benzyl alcohol, and thiomersal did not have a negative effect on the physiochemical properties of the lipid-based carriers and/or the RNA of the compositions. Moreover, as shown in Example 4, the antimicrobial preservatives (e.g. 2-PE) are still active in the presence of lipid-based carriers encapsulating the RNA. The data clearly demonstrates the suitability of said antimicrobial preservatives for multidose compositions or vaccines of the invention, e.g. a multidose vaccine against SARS-CoV-2.

### Example 5: In Vivo Preservative Comparability Design

In vivo studies were performed to investigate if the addition of the two aromatic alcohol preservatives (phenoxyethanol, benzyl alcohol) or thiomersal have a negative effect on the innate or the adaptive immune response Vaccines comprising lipid-based carriers encapsulating an RNA (R9515 encoding SARS-CoV-2; see Table 1) were generated according to Example 1. The different formulations were prepared by diluting a stock formulation using a 0.9% (w/v) NaCl solutions containing the antimicrobial preservatives phenoxyethanol, benzyl alcohol, or thiomersal. Before administration in vivo, the vaccines were incubated for up to 48 h (see “incubation time” in Table 10) after addition of the preservative to identify potential negative effects.

The study design is shown in Table 10. For each group, 8 female Balb/c mice were injected intramuscularly (i.m.). At day 1 (D1, 18 h post-vaccination), cytokine induction including IFN-alpha was evaluated from collected serum. At day 42, virus neutralizing titers (VNTs) and antigen-specific T-cells were evaluated.

TABLE 10
Study design of the in vivo vaccination experiment of Example 5

| | Antimicrobial | Incubation | | | |
|---|---|---|---|---|---|
| Gr. | preservative | time | | Dose/volume | Dosing |
| 1 | 0.5% phenoxyethanol | 48 | h | 2 μg/20 μl | Day 0, day 21 |
| 2 | 0.5% phenoxyethanol | 6 | h | 2 μg/20 μl | Day 0, day 21 |
| 3 | 0.5% phenoxyethanol | 0 | h | 2 μg/20 μl | Day 0, day 21 |
| 4 | 1% benzyl alcohol | 48 | h | 2 μg/20 μl | Day 0, day 21 |
| 5 | 1% benzyl alcohol | 6 | h | 2 μg/20 μl | Day 0, day 21 |
| 6 | 1% benzyl alcohol | 0 | h | 2 μg/20 μl | Day 0, day 21 |
| 7 | Thiomersal 0.001% | 48 | h | 2 μg/20 μl | Day 0, day 21 |
| 8 | Thiomersal 0.001% | 6 | h | 2 μg/20 μl | Day 0, day 21 |
| 9 | Thiomersal 0.001% | 0 | h | 2 μg/20 μl | Day 0, day 21 |
| 10 | No preservative control | 0 | h | 2 μg/20 μl | Day 0, day 21 |
| 11 | No mRNA control | 0 | h | 20 μl | Day 0, day 21 |

| | |
|---|---|
| 0.9% NaCL | Day 0, day 21 |

### Innate Immune Response:

Balb/c mice were vaccinated with the compositions of Table 10 and blood was sampled 18 h post-injection to investigate the effect on the induction of innate immune responses. PBMCs were stimulated with 10 μg/ml composition (see Table 10). After 24 h, supernatants were collected and analysed for cytokine release.

Mouse and human IFN-alpha was quantified using a human IFN-alpha ELISA according to manufacturer's instructions. Sera was diluted 1:1000 and 50 μl of dilution was tested. PBMC supernatant was used in a 1:10 dilution. IFN-gamma, IL-1beta, TNF, IL-4, IL-5, IL-6, and IL-13 were assessed using cytometric bead array (CBA) using the BD FACS CANTO II according to the manufacturer's instructions. Serum was diluted 1:3, and supernatants from PBMC stimulation were assessed undiluted.

As a result, it was observed that Cytokine induction (IFN-alpha, IFN-gamma, IL-1beta, TNF, IL-4, IL-5, IL-6, IL-13) was comparable between the compositions with preservative (group 1 to 9) in all tested condition (incubation for up to 48 h) and the composition without preservative (group 10).

### Adaptive Immune Response:

Virus neutralizing titers (VNTs) in serum samples taken on day 42 were determined in Vero E6 cells, where the cytopathic effect (CPE), meaning 50% death, was analyzed in microscopical or colorimetric reaction. The samples were measured in duplicates and neutralizing titers were defined as the highest sample dilution able to inhibit CPE. Starting dilution was 1:10. The tests were performed at Vismederi SRL, Siena, Italy.

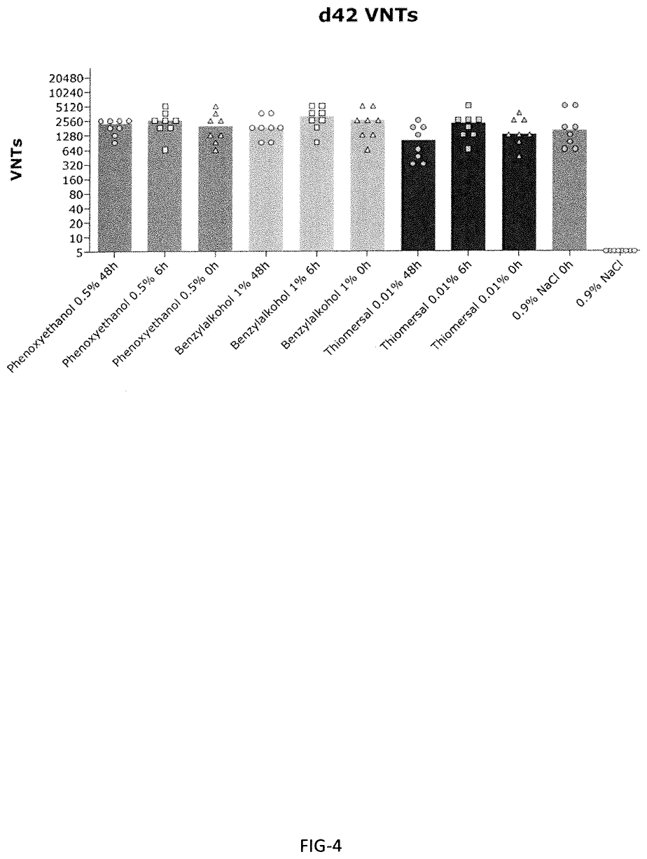

The results are shown in FIG. 4. As shown in FIG. 4, the VNTs induced upon injection with vaccines comprising antimicrobial preservatives were comparable to the VNTs induced with a vaccine that did not comprise an antimicrobial preservative (group 10, 0.9% NaCl). Moreover, incubation for up to 48 h with the respective preservative did not impair the potency of the vaccine to induce a strong adaptive immune response. However, more advantageous results could be observed for the aromatic alcohols (e.g. Phenoxyethanol), in particular when stored for 48 h. Nonetheless, the slight decrease in VNTs after 48 h storage with Thiomersal 0.01% 48 h (see FIG. 4) was still in an acceptable range.

In addition to VNTs, specific T-cell responses (CD4 and CD8 T cells) at day 42 were determined. Multifunctional IFN-gamma/TNF positive CD8 T cells and CD4 T cells in splenocytes isolated on day 42 were stimulated with a specific SARS-CoV-2 spike protein library for 24 h and intracellularly stained for cytokines (as commonly known in the art). As a result, it was observed that no significant differences were detectable between the group injected with vaccines comprising the preservative and the group that received a vaccine that did not comprise a preservative (group 10).

### SUMMARY

Based on the results of Example 5, the RNA-based vaccines comprising aromatic alcohols or thiomersal induced comparable innate and adaptive immune responses in vivo independent on the tested duration of storage time in the respective preservative. The data clearly shows that aromatic alcohols or thiomersal, in particular aromatic alcohols are suitable for preserving pharmaceutical composition comprising lipid-based carriers encapsulating an mRNA.

### Summary of the Findings of the Example Section

As shown herein, extensive studies have been conducted with various different antimicrobial preservatives to identify preservatives (aromatic alcohols, thiomersal) that were compatible with lipid-based carriers encapsulating an RNA. As demonstrated, aromatic alcohols (e.g. Phenoxyethanol) are the most favorable in the context of the invention as these preservatives did not affect the physiochemical or functional properties of the lipid-based carriers encapsulating an RNA.

1. A pharmaceutical composition for multidose administration comprising lipid-based carriers encapsulating an mRNA, wherein the composition comprises at least one antimicrobial preservative selected from at least one aromatic alcohol, wherein the pharmaceutical composition comprises more than one dose.

2. The pharmaceutical composition for multidose administration of claim 1, wherein the composition comprises 5 to 100 doses.

3. The pharmaceutical composition for multidose administration of claim 1 or 2, wherein one dose comprises an amount of RNA in a rage of 1 pg to 200 pg.

4. The pharmaceutical composition for multidose administration of claims 1 to 3, wherein the at least one aromatic alcohol is selected from phenoxyethanol, phenylethyl alcohol, benzyl alcohol, or a combination thereof.

5. The pharmaceutical composition for multidose administration of claims 1 to 4, wherein the at least one aromatic alcohol is phenoxyethanol.

6. The pharmaceutical composition for multidose administration of any one of the preceding claims, wherein the at least one aromatic alcohol is in a concentration of 0.1% (w/v) to 2% (w/v).

7. The pharmaceutical composition for multidose administration of any one of the preceding claims, wherein the composition comprises at least two antimicrobial preservatives selected from at least one aromatic alcohol and from at least one sugar alcohol.

8. The pharmaceutical composition for multidose administration of claim 7, wherein the at least one sugar alcohol is selected from xylitol, sorbitol, and/or glycerol.

9. The pharmaceutical composition for multidose administration of claim 7 or 8, wherein the at least one sugar alcohol is xylitol.

10. The pharmaceutical composition for multidose administration of claims 7 to 9, wherein the at least one sugar alcohol in a concentration of about 10 mM to about 200 mM.

11. The pharmaceutical composition for multidose administration of claims 7 to 10, wherein the at least one aromatic alcohol is phenoxyethanol and the least one sugar alcohol is xylitol.

12. The pharmaceutical composition for multidose administration of any one of the preceding claims, wherein the RNA has an RNA integrity of at least about 50%, preferably of at least about 60%, more preferably of at least about 70%, most preferably of at least about 80%.

13. The pharmaceutical composition for multidose administration of any one of the preceding claims, wherein the composition comprises less than about 20% free RNA, preferably less than about 15% free RNA, more preferably less than about 10% free RNA.

14. The pharmaceutical composition for multidose administration of any one of the preceding claims, wherein the RNA has a length ranging from about 200 nucleotides to about 10000 nucleotides, preferably wherein the RNA is at least 500 nt in length

15. The pharmaceutical composition for multidose administration of any one of the preceding claims, wherein the RNA comprises at least one coding sequence.

16. The pharmaceutical composition for multidose administration of claim 15, wherein the coding sequence encodes at least one peptide or protein suitable for use in treatment or prevention of a disease, disorder or condition.

17. The pharmaceutical composition for multidose administration of claim 16, wherein the at least one peptide or protein is selected or derived from an antigen or epitope of a pathogen, preferably selected or derived from a Coronavirus or a Rabies virus, or a fragment or variant of any of these.

18. The pharmaceutical composition for multidose administration of any one of the preceding claims, wherein the RNA comprises a 5′ cap structure, preferably a cap1 structure.

19. The pharmaceutical composition for multidose administration of any one of the preceding claims, wherein the wt/wt ratio of lipid to the RNA is from about 10:1 to about 60:1, preferably from about 20:1 to about 30:1, more preferably about 25:1.

20. The pharmaceutical composition for multidose administration of any one of the preceding claims, wherein the N/P ratio of the lipid-based carriers to the RNA is in a range from about 1 to about 10, preferably in a range from about 5 to about 7, more preferably about 6.

21. The pharmaceutical composition for multidose administration of any one of the preceding claims, wherein the lipid-based carriers have a polydispersity index (PDI) value of less than about 0.3, preferably of less than about 0.2, more preferably of less than about 0.1.

22. The pharmaceutical composition for multidose administration of any one of the preceding claims, wherein the lipid-based carriers have a Z-average size in a range from about 50 nm to about 150 nm, preferably in a range from about 50 nm to about 120 nm, more preferably in a range of about 60 nm to about 115 nm

23. The pharmaceutical composition for multidose administration of any one of the preceding claims, wherein the lipid-based carriers are liposomes, lipid nanoparticles, lipoplexes, and/or nanoliposomes.

24. The pharmaceutical composition for multidose administration of any one of the preceding claims, wherein the lipid-based carriers are lipid nanoparticles.

25. The pharmaceutical composition for multidose administration of any one of the preceding claims, wherein the lipid-based carriers comprise at least one aggregation-reducing lipid, at least one cationic lipid, at least one neutral lipid, and/or at least one steroid or steroid analog.

26. The pharmaceutical composition for multidose administration of claim 25, wherein the aggregation reducing lipid is a polymer conjugated lipid, e.g. a PEG-conjugated lipid.

27. The pharmaceutical composition for multidose administration of claim 26, wherein the polymer conjugated lipid is a PEG-conjugated lipid according to formula (IVa): 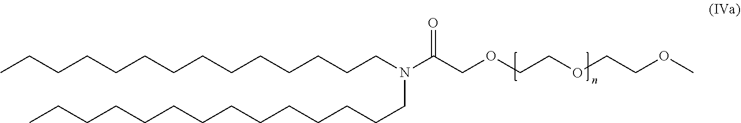
wherein n has a mean value ranging from 30 to 60, preferably wherein n has a mean value of about 45, 46, 47, 48, 49, 50, 51, 52, 53, 54, more preferably wherein n has a mean value of 49 or 45.

28. The pharmaceutical composition for multidose administration of claim 25 to 27, wherein the at least one cationic lipid is selected from a lipid according to formula III-3:

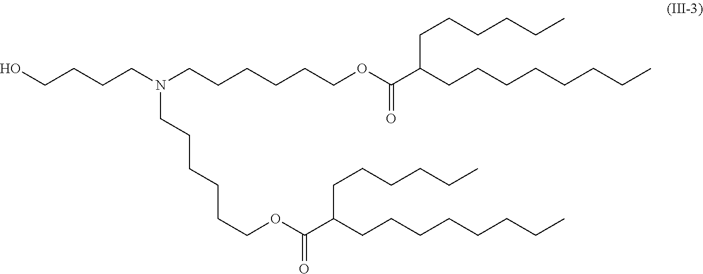 [SMILES: CCCCCCCCC(CCCCCC)C(=O)OCCCCCCN(CCCCO)CCCCCCOC(=O)C(CCCCCC)CCCCCCCC]

29. The pharmaceutical composition for multidose administration of claim 25 to 28, wherein the at least one neutral lipid is 1,2-distearoyl-sn-glycero-3-phosphocholine (DSPC).

30. The pharmaceutical composition for multidose administration of claim 25 to 29, wherein the steroid or steroid analog is cholesterol.

31. The pharmaceutical composition for multidose administration of any one of the preceding claims, wherein the lipid-based carrier comprises
i. at least one cationic lipid, preferably as defined in claim 28;
ii. at least one neutral lipid, preferably as defined in claim 29;
iii. at least one steroid or steroid analogue, preferably as defined in claim 30; and
iv. at least one aggregation reducing lipid, preferably as defined in claim 26 or 27.

32. The pharmaceutical composition for multidose administration of claim 31, wherein (i) to (iv) are in a molar ratio of about 20-60% cationic lipid, about 5-25% neutral lipid, about 25-55% steroid or steroid analog, and about 0.5-15% aggregation reducing lipid.

33. The pharmaceutical composition for multidose administration of claim 31 or 32, wherein (i) to (iv) are in a molar ratio of about 47.4% cationic lipid, 10% neutral lipid, 40.9% steroid or steroid analogue, and 1.7% aggregation reducing lipid.

34. The pharmaceutical composition for multidose administration of any one of the preceding claims, further comprising a sugar in a concentration of about 5 mM to about 300 mM, preferably sucrose in a concentration of about 14 mM.

35. The pharmaceutical composition for multidose administration of any one of the preceding claims, further comprising a salt in a concentration of about 10 mM to about 300 mM, preferably NaCl in a concentration of about 150 mM.

36. The pharmaceutical composition for multidose administration of any one of the preceding claims, wherein the composition is free of virus particles and/or wherein the composition does not comprise and added adjuvant.

37. The pharmaceutical composition for multidose administration of any one of the preceding claims, wherein the composition has been formulated by adding the at least one antimicrobial preservative to a composition comprising lipid-based carriers encapsulating an RNA.

38. The pharmaceutical composition for multidose administration of any one of the preceding claims, wherein the composition is stable for at least about 1 day after formulation of the composition.

39. The pharmaceutical composition for multidose administration of any one of the preceding claims, wherein the composition is stable for at least about 1 day after a first dose withdrawal.

40. The pharmaceutical composition for multidose administration of any one of the preceding claims, wherein after a first dose withdrawal and/or after formulation of the composition, the integrity of the RNA decreases less than about 30%, preferably less than about 20%, more preferably less than about 10%.

41. The pharmaceutical composition for multidose administration of any one of the preceding claims, wherein after a first dose withdrawal and/or after formulation of the composition, the amount of free RNA does not increase by more than 10%, preferably by not more than 5%.

42. The pharmaceutical composition for multidose administration of any one of the preceding claims, wherein after a first dose withdrawal and/or after formulation of the composition, the PDI value of the lipid-based carriers encapsulating the RNA does not increase by more than a value of about 0.2, preferably by not more than a value of about 0.1.

43. The pharmaceutical composition for multidose administration of any one of the preceding claims, wherein after a first dose withdrawal and/or after formulation of the composition, the Z-average size of the lipid-based carriers encapsulating the RNA does not increase by more than 20%, preferably by not more than 10%.

44. The pharmaceutical composition for multidose administration of any one of the preceding claims, wherein after a first dose withdrawal and/or after formulation of the composition, the potency of the composition decreases less than about 30%, preferably less than 20%, more preferably less than 10%.

45. The pharmaceutical composition for multidose administration of claims 40 to 44, wherein the parameters are determined in comparison to a reference composition that does not comprise the preservative.

46. The pharmaceutical composition for multidose administration of any one of the preceding claims, wherein the composition is microbially preserved for at least about 1 day, preferably for at least about 1 day after a first dose withdrawal

47. The pharmaceutical composition for multidose administration of any one of the preceding claims, wherein the composition is microbially preserved at a temperature of about 5° C. to about 25° C.

48. A vaccine for multidose administration comprising or consisting of a pharmaceutical composition for multidose administration of any one of claims 1 to 47.

49. The vaccine for multidose administration of claim 48, wherein the vaccine is against a Coronavirus, preferably against SARS-CoV-2.

50. The vaccine for multidose administration of claim 48 or 49, wherein the vaccine is against a pandemic virus.

51. A kit or kit of parts for preparing and/or administering a multidose composition or vaccine wherein the kit comprises the following components
(A) at least one pharmaceutical composition comprising lipid-based carriers encapsulating an RNA; and
(B) at least one sterile buffer for diluting component A, wherein the sterile dilution buffer comprises at least one antimicrobial preservative selected from at least one aromatic alcohol.

52. The Kit or kit of parts of claim 51, wherein the multidose composition or vaccine is a pharmaceutical composition for multidose administration as defined in claims 1 to 47, or a vaccine for multidose administration as defined in claims 48 to 50.

53. The kit or kit of parts of claim 51 or 52, wherein component A and component B are provided in separate containers or vials.

54. The kit or kit of parts of claim 51 to 53, wherein component A and component B are combined to obtain a diluted pharmaceutical composition or vaccine for multidose administration.

55. The Kit or kit of parts of claim 54, wherein the dilution factor is in a range from 1:1 to 1:50, preferably between 1:5 and 1:15.

56. The kit or kit of parts of claim 51 to 55, wherein component B comprises at least one antimicrobial preservative as defined in claims 4 to 11.

57. The kit or kit of parts of claim 51 to 56, wherein component B comprises at least one aromatic alcohol, preferably phenoxyethanol and, optionally, at least one sugar alcohol, preferably xylitol.

58. The kit or kit of parts of claim 51 to 57, wherein component B comprises a salt, preferably NaCl, optionally in a concentration of about 0.9%.

59. The kit or kit of parts of claim 51 to 58, wherein component B is a heat autoclaved sterile buffer.

60. The kit or kit of parts of claim 51 to 59, wherein the lipid-based carriers of component A are as defined in claims 19 to 33.

61. The kit or kit of parts of claim 51 to 60, wherein the RNA of component A is as defined in claims 12 to 18.

62. The kit or kit of parts of claim 51 to 61, wherein component A comprises a sugar in a concentration of about 50 mM to about 300 mM, preferably sucrose in a concentration of about 150 mM.

63. The kit or kit of parts of claim 51 to 62, wherein component A comprises a salt in a concentration of about 10 mM to about 200 mM, preferably NaCl in a concentration of about 75 mM.

64. The Kit or kit of parts of claim 51 to 63, wherein the kit or kit of parts comprises at least one means for combining component A and component B to obtain the multidose composition or vaccine.

65. Kit or kit of parts of claim 51 to 64, wherein the kit or kit of parts comprises at least one means for administering the multidose composition or vaccine.

66. Kit or kit of parts of claim 51 to 65, wherein, after combining component A and component B, the integrity of the RNA decreases less than about 30%, preferably less than about 20%, more preferably less than about 10%.

67. Kit or kit of parts of claim 51 to 66, wherein, after combining component A and component B, the amount of free RNA does not increase by more than 10%, preferably by not more than 5%.

68. Kit or kit of parts of claim 51 to 67, wherein, after combining component A and component B, the PDI value of the lipid-based carriers encapsulating the RNA does not increase by more than a value of about 0.2, preferably by not more than a value of about 0.1.

69. Kit or kit of parts of claim 51 to 68, wherein, after combining component A and component B, the Z-average size of the lipid-based carriers encapsulating the RNA does not increase by more than 20%, preferably by not more than 10%.

70. Kit or kit of parts of claim 51 to 69, wherein, after combining component A and component B, the obtained composition or vaccine is microbially preserved, preferably for at least about 1 day and/or at a temperature of about 5° C. to about 25° C. and/or for at least about 1 day after a first dose withdrawal.

71. The pharmaceutical composition for multidose administration of any one of claims 1 to 47, the vaccine of claim 48 to 50, the kit or kit of parts of any one of claim 51 to 69, for use as a medicament.

72. The pharmaceutical composition for multidose administration of any one of claims 1 to 47, the vaccine of claim 48 to 50, the kit or kit of parts of any one of claim 51 to 69, for use in the treatment or prophylaxis of an infection with a pathogen or of a disorder related to such an infection, preferably wherein the pathogen is a Coronavirus.

73. A method of treating or preventing a disorder, wherein the method comprises applying or administering to a subject in need thereof the pharmaceutical composition for multidose administration of any one of claims 1 to 47, the vaccine of claim 48 to 50, the kit or kit of parts of any one of claim 51 to 69.

74. A method of treating or preventing a disorder of claim 73, wherein the disorder is an infection with a pathogen, preferably an infection with a Coronavirus.

75. A method of formulating a multidose composition or vaccine comprising:
a) obtaining a first component comprising lipid-based carriers encapsulating an RNA;
b) obtaining a second component comprising at least one antimicrobial preservative selected from at least one aromatic alcohol; and
c) mixing said first and second components to formulate a multidose composition or vaccine.

76. The method of claim 75, wherein the first component is a liquid composition.

77. The method of claim 75, wherein the first component is lyophilized or spray-dried composition.

78. The method of claims 75 to 77, wherein the lipid-based carriers of the first component are as defined in claims 19 to 33.

79. The method of claims 75 to 78, wherein the RNA of the first component is as defined in claims 12 to 18.

80. The method of claims 75 to 79, wherein the antimicrobial preservative of the second component are as defined in claims 4 to 11.

81. The method of claim 75 to 80, wherein the multidose composition or vaccine is a composition or vaccine as defined in any one of claims 1 to 47 or claims 48 to 50.

82. Use of an aromatic alcohol for preserving and/or formulating a composition or vaccine comprising lipid-based carriers encapsulating an RNA.
